(19) United States
(12) Patent Application Publication
(10) Pub. No.: US 2026/0337695 A1
(43) Pub. Date: Oct. 29, 2026

(54) METHYLATION PROXIMITY SEQUENCING

(71) Applicant: NCAN Genomics, Inc., Vancouver (CA)

(72) Inventor: Joel Pel, Vancouver (CA)

(21) Appl. No.: 19/648,176

(22) Filed: Apr. 15, 2026

Related U.S. Application Data
(60) Provisional application No. 63/789,622, filed on Apr. 16, 2025.

(51) Int. Cl. C12Q 1/6886

(52) U.S. Cl. CPC C12Q 1/6886; C12Q 2600/154; C12Q 2600/156

(57) ABSTRACT

The invention provides methods for determining patterns of genetic and epigenetic modifications in genetic material. Methods of the invention use proximity sequencing, in which two polynucleotides that are independently obtained from fragments of DNA are linked together, keeping those polynucleotides together for chemical or enzymatic treatment, amplification to prepare a sequencing library, and sequencing. Due to the chemical or enzymatic treatment, different base modifications give different base calls after sequencing, and by applying the logic of what base calls would be expected, methods of the invention may provide a series of “base calls” including the epigenetic modifications for the fragment of DNA.

### REFERENCE TO A “SEQUENCE LISTING”

A “Sequence Listing XML” is submitted herewith in XML file format and (i) the name of the file is BRL-028-01-Seqs.xml; (ii) the date of creation is Apr. 14, 2026; and (iii) the size of the file is 10,394 bytes and the material in the XML file is incorporated by reference.

### BACKGROUND

The sequence of a genome is nominally made up of adenine (A), cytosine (C), guanine (G), and thymine (T) bases and the central dogma of molecular biology holds that the sequence of those bases encode the sequence of amino acids that make up the proteins necessary for all living systems. A protein is encoded by a gene—a set of those bases in an order that encodes the gene. The gene is translated and transcribed to express the protein according to the needs of a cell. Different proteins will be expressed at different developmental stages and in different tissues. For example, red blood cells would be expected to express abundant hemoglobin proteins while neurons would express voltage-gated ion channels. It is conventional wisdom of biology that the sequence of bases in the genome constitutes the genetic information that is inherited.

Epigenetic modification refers to categories of molecular changes to genetic material such as the methylation of cytosine bases that may occur in DNA as well as modification of proteins, such as histone acetylation, associated with that DNA. There are a number of significant epigenetic modifications that are known, which include 5-methyl-cytosine (5mC) and 5-hydroxymethyl cytosine (5hmC). Epigenetic modifications are important in developmental biology and organismal health because those modifications influence protein expression. In the simplest example, it is thought that DNA methylation inhibits expression so that, in some cell and tissue types, genes for important constituent proteins will be hypomethylated while other genes may be hypermethylated, and regulatory pathways of the cell can change methylation patterns according to various biochemical influences. In one example, there are genes that would be causative of unchecked cellular proliferation, or cancer, except that those genes are maintained in most cells in a hypermethylated state. Epigenetic modifications are also important in biology because patterns of differential modification are inherited. By inheriting patterns of differential methylation, progeny can inherit traits beyond those that are only encoded in the order of A, C, G, and T bases of DNA.

### SUMMARY

The invention provides methods for determining patterns of epigenetic modifications in genetic material. Methods of the invention use proximity sequencing, in which two polynucleotide strands that are independently obtained from a fragment of DNA are linked together. The linked strands may be treated so that certain epigenetic modifications are identified when the strands are sequenced. For example, one of the linked strands may be single-stranded while the other is duplexed and the linked strands may be exposed to enzymes that methylate and protect certain cytosines only in the duplex portion, after which unprotected cytosines are converted to uracil and read as thymine during sequencing. After such treatment, methylated cytosines in the original fragment appear as C base calls across all sequence reads of the original fragment, and un-modified cytosines appear as T base calls among the sequence reads originating from copied templates. Not only do the linked strands, obtained from the same fragment, allow sequencing to identify base modifications, the linked, independent strands from the one fragment also provide for the high sensitivity and specificity detection of genetic variants (e.g. SNVs and indels) through the capability of duplex error correction. When those linked strands are amplified, any polymerase error would appear in only a limited subset of the amplicons while a true mutation will appear in all of the amplicons. When those amplicons are sequenced, the sequence reads represent the base composition of the fragment except that any polymerase or sample preparation errors will appear in fewer than all of the sequence reads and may be corrected for. Accordingly, the use of at least two linked strands, obtained independently from one DNA fragment, provides for a multi-modal readout. By subjecting the linked strands to base-conversion reactions before amplification and sequencing, the base calls among sequence reads can indicate both the bases and the base modifications of the original fragment. By conducting one sequencing reaction on two strands that are from the same fragment and have the same base composition in the same orientation, base damage or polymerase error during the amplification and sequencing will manifest as non-uniform base calls among the sequence reads. Identifying those non-uniform base calls among the sequence reads provides a mechanism by which to correct for DNA damage or polymerase error in the sample or sample preparation steps. By those means, the sequence reads from the linked strands provide a readout with multi-modal information about the fragment and sample preparation steps. Errors introduced by sample handling may be corrected for and sequence reads may be interpreted to provide a description of bases and base modifications of the original sample.

Preferred embodiments use versions of methyl-sequencing workflows to identify modified bases such as modified cytosines within DNA of a sample. Where, for example, 5mC and 5hmC are of interest, methods of the invention may be used to analyze a fragment of DNA and, after sequencing, provide base calls that indicate six different possibilities for each position of a strand of the fragment, where those six different possible base calls are A, C, 5mC, 5hmC, G, and T. Extensions and variations are within the scope of the invention, but the use of proximity sequencing with methylation-specific chemical or enzymatic treatments allows for the various base calls to be made with single-base resolution according to methods of the invention.

Proximity sequencing (Pro-Seq) describes workflows in which two different strands with the same base sequence are obtained independently from a fragment and are linked together and used to seed one single sequencing reaction. Each strand may be treated differently. For example, one strand may be in double-stranded form while the other is single stranded. Those linked strands (hereinafter, a “complex”) may be exposed to enzymes or chemicals that react differently with the single- and the double-stranded strands. Subjecting the two linked identical strands, in which one is double stranded while the other is single-stranded, to a set of methyl-sequencing reactions provides a pair of linked strands that may differ at positions where the original fragment included a modified base. The complex is used to seed a single sequencing reaction. For example, the linked strands of the complex may be exposed to primers (e.g., such as a plurality of clonal primers on a solid substrate such as a flow cell or a bead) and amplified to create a cluster or amplicons in which a position corresponding to the modified base in the original fragment contains mixed bases among the amplicons (e.g., a methylated C in the original fragment may be represented roughly 50/50 by C and T in the amplicons after enzymatic de-amination of C to U only in single-stranded portions of the complex). Because the complex represents more than one strand and is used to seed a single sequencing reaction, when the cluster is sequenced to yield sequence reads, the sequence reads will have a majority single base consensus at positions that were not modified, and individual reads will show mixed base calls (“C and T”) for positions that were modified. The logic of base calling is extended herein to specifically call 5mC, 5hmC, or others using enzymes and treatments discussed herein.

In proximity sequencing, amplicons are generated from a complex comprising two or more linked strands that are independently obtained from one fragment. Because the linked strands are obtained independently (e.g., from a sense and an antisense strand) and the cluster is used to seed a single sequencing reaction, Pro-Seq provides a mechanism for duplex error correction. Because one strand is made with polymerase reactions that did not contribute to making the other strand, when those two linked strands are amplified, any polymerase error will be restricted to a limited number of amplicons. There is no opportunity for a polymerase error to create a base that appears across all amplicons. For example, if a polymerase incorporates an A where a G would be expected, some fraction (e.g., half), but not all, the amplicons will have an A in that position. After the amplicons are sequenced, the same fraction of sequence reads will show A at that position. An associated analysis unit can detect that mixture of base calls and flag the base call as likely polymerase error. In preferred embodiments of proximity sequencing, two linked strands represent a sense strand of a fragment and a corresponding antisense strand of the fragment. For example, using workflows shown herein, the sense strand of a fragment may be linked to a copy of the antisense strand of that fragment. Those two strands will have the same order of bases and be linked “in phase”, i.e., in the same 5′ to 3′ orientation. Having those two strands linked together in proximity to each other allows the two linked strands to be used to seed a single sequencing reaction. For example, the two linked strands may be captured by primers bound to a flow cell or bead and those bound primers may be extended, copying the two linked strands, to form one cluster used in a single sequence reaction. Because those two linked strands are kept in proximity to each other and used to seed one cluster for sequencing, polymerase errors can be corrected for in the sequence reads. Thus, proximity sequencing offers built-in duplex error correction.

By combining versions of methylation sequencing with proximity sequencing (methyl Pro-Seq, or mPS), not only do mPS methods of the invention give built-in duplex error correction for discrimination of polymerase errors, DNA lesions, and sequencing errors from true genomic variants, methods of the invention provide for the simultaneous detection of modifications, and patterns of modifications, in a single sequencing readout. Methods of the invention are useful to specifically detect and distinguish between 5mC and 5hmC in nucleic acids. Methods of the invention are further useful to identify phased variants. Due to the sensitivity and specificity of the proximity sequencing and its built-in duplex error correction, true variants are found and are distinguished from polymerase errors and other DNA lesions. Thus when two true variants are found on one DNA fragment, those variants are known to arise from the same chromosome and thus be in cis, in the sense of haplotype phasing. Such methods are of particular value when tumor DNA is being analyzed alongside matched normal DNA and sequence reads. When comparison of a true variant found in tumor sequence reads to matched normal sequence reads reveals the variant to be a tumor-specific variant, the detection of tumor-specific phased variants provides a tumor-specific genetic signature that is exceedingly improbable to be recapitulated by chance, or independently in another patient, or by polymerase error. Here, phased variant detection includes the detection of modified bases and their phasing (e.g., in-phase modified bases). Noting that methyl proximity sequencing as disclosed herein provides base calls for a DNA fragment potentially using more than four base identifies, e.g., using A, C, 5mC, 5hmC, G, and T, methods here detect modification variants as one type of variant. Where more than one modified bases are found on a tumor DNA fragment, those are tumor-specific, phased modification variants. Phased variants can include SNPs that involve only the 4 unmodified bases and/or any epigenetic modification to a base. For example, analysis of a tumor fragment may reveal a rare, true variant SNP, such as an A to G substitution in phase with (in cis with) one or more modified bases such as 5mC and/or 5hmC. Thus it can be seen that the identifies of epigenetic modifications as detected and shown using methods herein may be used to provide information about phased variants, which may include multiple modified bases or SNPs and/or small InDels in phase with any modified bases. Detecting phased variants in tumor DNA provides an exquisitely specific and sensitive tumor marker due to the extreme improbability of such a combination of features to arise independently.

Methods of the invention may be used to analyze any suitable sample that includes a double-stranded DNA fragment such as sample from a particular cell or tissue type including from a tumor. For example, a formalin-fixed, paraffin embedded tumor slice may be analyzed to determine tumor-specific patterns of modification. In another example, a tissue or other sample, such a liquid biopsy sample, may be subject to methylation proximity sequencing to detect patterns of modification among genes of the subject. The detected patterns may provide a tool for early cancer detection or other disease detection. Where tumor-specific patterns of modification have previously been determined, methods of the invention may be “tumor informed” and may be used to stage the cancer or evaluate treatment results or to detect minimal residual disease (MRD) or for early cancer detection (ECD). Thus profiling differential modification in genetic material using methyl proximity sequencing (mPS) as disclosed herein provides approaches useful in tumor-informed MRD and ECD assays. In such tumor-informed assays using mPS, tumor DNA is sequenced and analyzed to identify genomic variants and epigenomic markers which are subsequently looked for in cell-free DNA.

In certain aspects, the disclosure provides methods that include ligating a fragment to a linking adaptor in which a first oligo is linked to a primer annealed to a second oligo, whereby the first oligo is ligated to a first strand of the fragment, extending the primer to make a copy of a second strand of the fragment, resulting in a molecular complex that includes: the first strand ligated to the first oligo, the first oligo linked to the primer wherein the primer is extended into a product that includes a copy of the second strand, and the second strand annealed to the product and ligated to the second oligo, methylating a cytosine base (C) in the copy of the second strand with a reagent composition that acts only on double-stranded DNA and does not modify the single-stranded first strand; and converting mC and hydroxymethyl cytosine (hmC) in the complex into dihydrouracil (DHU). The linking adaptor may attach a first sequencing primer binding site to the first stand and a second sequencing primer binding site to the copy of the second strand and the method may include forming a cluster from the complex and conducting a first sequencing reaction using a first sequencing primer to obtain a first read for the first strand and conducting a second sequencing reaction using a second sequencing primer to obtain a second read for the copy of the second strand. The methylating step may be performed using a methyltransferase enzyme that methylates unmodified C in hemi-methylated CpG dinucleotides. The converting step may use ten-eleven translocase (TET) enzymes and/or pyridine borane.

Methods may include seeding a sequencing reaction with the complex by capturing the first strand and the copy of the second strand with capture primers. The capture primers may be linked to a solid support (e.g., flow cell, bead, or dendrimer) and the method may include conducting an amplification reaction to form a cluster comprising a plurality of amplicons representing both the first stand and second strand of the fragment. Methods may include conducting the sequencing reaction using a sequencing system connected to an analysis unit to generate sequence data representing the fragment, wherein the analysis unit (e.g., software module of the sequencing system or in a connected computer) makes base calls for the first strand based on sequencing data read from the first strand and from the copy of the second strand. The analysis unit calls two adjacent positions in the first strand as: (i) GC when sequencing data read for corresponding positions from the first strand and from the copy of the second strand include GC and GC, respectively, (ii) GmC when sequencing data read for corresponding positions from the first strand and from the copy of the second strand include GT and GT, respectively, and (iii) GhmC when sequencing data read for corresponding positions from the first strand and from the copy of the second strand include GT and GC, respectively.

In some embodiments, after the converting step the first strand and the copy of the second strand are linked together at 5′ regions thereof and have the same sequence of bases in the same order except that C has been converted to DHU in some places. Methods may include—prior to the ligating step—end-repairing and A-tailing a plurality of fragments that include the fragment obtained from a sample (such as a blood or plasma sample obtained by liquid biopsy, a tumor sample, or other sample). The plurality of fragments may be used in a whole genome sequencing (WGS) process to identify sequences of, and positions of epigenetic modifications within, the plurality of fragments from the sample. In some embodiments, positions of epigenetic modifications in tumor DNA from a tumor have previously been determined for a subject from whom the sample was obtained and the method includes detecting the epigenetic modifications among the plurality of fragments from blood or plasma from the sample to detect the tumor DNA in the sample.

Methods may include amplifying the first strand and the copy of the second strand to yield amplification products in which DHU is represented as a T. In some embodiments, in the amplification products, any unmodified C present in the first strand of the fragment is represent as a C.

The fragment may be from a sample from a subject and method may include identifying patterns of cytosine modification (where cytosine modifications comprise methylation and hydroxymethylation) at genetic loci of the subject. The sample may be from a tumor and the fragment may be tumor DNA and the identifying step may provide tumor-specific patterns of cytosine modification. Methods may include comparing the tumor-specific patterns of cytosine modification to a cancer database or atlas for the detection of evidence of cancer in the subject. Methods may include subsequently assaying a post-treatment sample from the subject for persistence of the patterns of cytosine modification as evidence of minimal residual disease. In certain embodiments, the sample comprises a formalin-fixed, paraffin embedded (FFPE) slice of tumor tissue or a tumor biopsy and the post-treatment sample may use a liquid biopsy sample and the assaying step may involve a WGS analysis of cell-free DNA in the liquid biopsy sample.

In certain aspects, the invention provides a method that includes ligating a fragment to a linking adaptor in which a first oligo is linked to a primer annealed to a second oligo, wherein the first oligo is ligated to a first strand of the fragment and extending the primer to make a copy of a second strand of the fragment, resulting in a complex that includes: the first strand ligated to the first oligo, the first oligo attached to the primer wherein the primer is extended into a product that includes a copy of the second strand, and the original second strand annealed to the product and ligated to the second oligo. Accordingly the complex has a single-stranded portion (the first, or forward strand) and a potentially double-stranded portion (the copy of the second, or reverse, strand annealed to the second strand). The method may include an optional step of protecting 5hmC by glycosylation so that, in a downstream step, the hydroxy-methyl modification is not copied by a DNMT enzyme (e.g., DNMT5) from the second strand to the copy of the second strand, thereby allowing sequencing results to distinguish 5hmC from C and 5mC. The method may optionally include introducing a DNA methyltransferase (e.g., DNMT1 or DNMT5) that methylates C bases that are in the copy of the second strand in double-stranded form and present in hemi-methylated CpG dinucleotides. If the first strand is single-stranded, then a cytosine present in the first strand will not be methylated by a DNMT enzyme.

In some embodiments, the method includes converting a cytosine in the first strand into a uracil. This may be done using a deaminase that does not act on double-stranded DNA. The copy of the second strand in its duplex structure may include a matched cytosine at a position corresponding to a position of the first cytosine in the first strand.

Methods may include a step of converting cytosine to uracil by deamination. The converting step may be performed using an enzyme that acts only on bases in single-stranded DNA. For example, the converting step may be performed using apolipoprotein B mRNA editing enzyme, catalytic polypeptide-like (APOBEC).

In certain embodiments, the complex is subject to denaturing conditions so that the copy of the second strand is single-stranded. With a denaturing step, DNMT enzyme does not act on the single-stranded copy of the second strand. In those certain embodiments, a C in the single stranded copy of the second strand may be converted to U.

For the conversion to U, any suitable conversion method may be used including, for example, chemical or enzymatic conversion. For enzymatic conversion in some embodiments, methods may include protecting at least one modified cytosine in the first strand from conversion to uracil. Protecting modified cytosines may involve the use of a ten-eleven translocation (TET) enzyme and/or a beta-glucosyltransferase (β-GT). A TET enzyme may be used to convert 5mC to 5hmC and β-GT may be used to glycosylate 5hmC yielding 5gmC, which is not converted to uracil by APOBEC. Other approaches and workflows are within the scope of the invention including, for example, bisulfite conversion.

Where the original fragment includes a methylated CpG dinucleotide, treatment of the complex with DNMT followed by TET enzymes and β-GT leaves a 5gmC in the copy of the second strand that is not de-aminated by APOBEC.

Methods may include sequencing the first strand to yield first sequence reads and the copy of the second strand to yield second sequence reads. In some embodiments (where the copy of the second strand was not denatured), the first sequence reads represent the first cytosine as T and the matched cytosine as C. The enzymatic (or chemical) treatments and optional denaturing steps may be combined in different orders to produce different base-calls in the resultant sequence reads. Combinations of process steps and associated resulting base calls, and what base modifications are indicated by those base calls, are summarized below herein. An important feature by which a mix of base calls among sequence reads is informative about modified bases in the template is the molecular linkage keeping one strand of the template in proximity to a copy of the other strand of the template.

In preferred embodiments, the first strand and the copy of the second strand are linked together at 5′ regions thereof and have the same sequence of bases in the same order except where enzymatic treatments have created different bases, or bases with different modifications, within the first strand and the copy of the second strand. For example, where DNMT is used to methylate a C in the copy of the second strand at a position corresponding to an unmodified C in the first strand, after treatment with APOBEC, the uracil in the first strand corresponds to a matched (modified) cytosine in copy of the second strand.

Methods may include—prior to the converting step—methylating, with a methyltransferase, a second cytosine that is in the copy of the second strand, base-paired with a methylated CpG dinucleotide in the second strand, to yield a 5mC in the copy of the second strand. These embodiments may optionally include—prior to this methylating step—glycosylating (or “protecting”) 5hmC. If 5hmC is glycosylated, DNMT5 may be used to copy 5mC but not 5hmC modifications from the second strand to the copy of the second strand. If the optional protecting step is not performed, then a DNMT enzyme may be used to copy 5mC and 5hmC modifications from the second strand to the copy of the second strand. Methods may include amplifying the first strand and the copy of the second strand to yield amplification products in which the uracil is represented as a T and the second cytosine is represented as a C.

The linkage of one strand of the template to a copy of the other strand of the template is one example of a workflow for proximity sequencing, which links together more than one molecule, each molecule being from a fragment or a copy independently made from the fragment, into a complex. Two nucleic acid molecules are copies made independently of each other when any synthesis reaction used the pathway for making one of the molecules was not also used in the pathway for making the other of the two molecules.

Proximity sequencing (Pro-Seq) uses at least two linked molecules, each molecule being a strand of a fragment or a copy independently made from the fragment, in which the at least two linked molecules have essentially the same sequence of bases in the same orientation (i.e., are not reverse or complementary to each other with respect to the linkage). Those linked molecules form a complex and the complex is used as an input for a sequencing reaction, including any associated amplification reaction. An example of a complex that meets the requirement for proximity sequencing is a “forward” (or first or sense) strand of a DNA fragment that is linked to a copy of what was the reverse complementary “reverse” (or second or antisense) strand of that fragment, in which the forward strand and the copy of the reverse strand are linked by a linker via their 5′ ends. The skilled artisan would expect those molecules to have the same series of bases, except that base modifications or polymerase error may create slight differences (hence, having “essentially” the same sequence). Where (i) chemical or enzymatic treatment of unmatched based modifications, or (ii) polymerase error imbue the strands with dissimilar bases at corresponding positions, copying by polymerase will create amplicons that do not match at that position. Sequencing those amplicons will generate sequence reads with a mixture of base calls for that position. The mixture of base calls, and the knowledge of the chemical or enzymatic treatment of unmatched based modification, provide tools by which to report the presence of the base modifications in the original fragments. Methods herein are specifically addressed to identifying methylated cytosine (5mC) and hydroxymethylated cytosine (5hmC), among others, in nucleic acids using Pro-Seq and may be referred to as methyl-ProSeq or mPS.

Preferred embodiments of mPS use a complex comprising a first strand from a fragment linked to a copy of a second strand, wherein chemical and/or enzymatic treatment of unmatched base modifications leaves dissimilar bases at corresponding positions in first strand and the copy of the second strand. Methods may include amplifying the first strand and the copy of the second strand using a plurality of primers linked to a solid support (e.g., surface of a flow cell or bead) to form a cluster on the solid support. In some embodiments, the solid support is a bead and the amplification is performed in an aqueous compartment such as a well or droplet. Methods may include conducting a sequencing reaction on the cluster to generate sequence reads, wherein the sequence reads contain (i) a mixture of T and C base-calls at a position corresponding the first cytosine, and (ii) C base calls for the second cytosine. Depending on process steps used to treat the complex, the sequence reads will include different combinations of base calls for certain modified bases in the original fragment.

Methods may include mapping the sequence reads to reference data (such as a published human genome or cancer atlas) to identify genes and patterns of methylation or other modification within those identified genes. The identified patterns of modification may be used for disease detection. For example, there are known associations in the literature between cancer onset and patterns of differential methylation. Methods of the invention may be used to detect and document methylation patterns and may be used for early evidence or detection of cancer. Methods of the invention may be used with a sample comprising tumor DNA such as nucleic acid extracted from a tumor sample such as a formalin-fixed, paraffin embedded (FFPE) tissue slice. Methods may be used to analyze or sequence tumor nucleic acid, and identify mutations and modifications that are specific to that tumor. Methods may further be used to show patterns of modification (e.g., hypo- or hyper-methylation) among genes in that tumor, optionally in comparison to “matched normal”, i.e., to show patterns of differential methylation characterizing that tumor. Where tumor-specific differential methylation patterns are known, methods of the invention may be used for tumor detection from a clinical sample. For example, a liquid biopsy, blood draw, or other clinical sample may be obtained and cell-free DNA may be analyzed by methods herein. Methods here may be used to detect tumor-specific differential methylation patterns as evidence of, e.g., minimal residual disease after treatment.

As discussed, methods may include deaminating at least one cytosine in the first strand of the complex to leave a uracil base. Methods may further include protecting cytosines in the molecular complex from conversion to uracil; exposing the complex to a methyltransferase to methylate a second cytosine that is in the copy of the second strand, base-paired with a methylated CpG dinucleotide in the second strand, to yield a 5mC in the copy of the second strand; and sequencing the first strand and copy of the second strands to yield sequence reads, wherein base calls among the sequence reads reveal positions of cytosine methylation in the fragment. The fragment may be from a sample from a subject and method may include mapping the sequence reads and the positions of cytosine methylation to reference data and identifying patterns of cytosine methylation in genetic loci of the subject.

In other aspects, the invention provides methods that include exposing a fragment to adaptors that include (i) a linking adaptor comprising a double-stranded portion in which a 3′ end of a first oligo is annealed to a 5′ end of a second oligo, wherein the first oligo is linked to a primer complementary to the second oligo, and (ii) a second adaptor comprising a third oligo annealed to a fourth oligo. Methods include ligating the 3′ end of the first oligo to a first strand of the fragment, the 5′ end of the second oligo to a second strand of the fragment, a 5′ end of the third oligo to the first strand, and a 3′ end of the fourth oligo to the second strand. Methods further include extending the primer, annealed to the second oligo, to form an extension product that includes a copy of the second strand and the fourth oligo, thereby forming a complex for Pro-Seq. The complex includes the first oligo ligated to the first strand and the third oligo, with first oligo linked to the primer and the extension product, wherein the first strand and the copy of the second strand have the same sequence of bases except that any C, 5mC, or 5hmC in the first strand is a C in the copy of the second strand. Such methods may include annealing a universal primer to the third oligo and extending the universal primer to form a copy of the first strand annealed to the first strand, such that the complex includes the first strand annealed to the copy of the first strand and the copy of the second strand annealed to the second strand. In an optional “protecting” step, 5hmC modifications in the original fragment are protected by glycosylation so that the 5hmC modifications are not copied in a subsequent methyltransferase step (only 5mC modifications will be). The complex is treated with a methyltransferase to methylate a cytosine in the copy of the second strand paired with a methylated CpG dinucleotide in the second strand (and to copy 5hmC when the optional protecting step is not performed). Methods may include protecting modified cytosines in the first strand and the copy of the second strand and enzymatically converting unmodified cytosines to uracil. Methods may include conducting a sequencing reaction with the first strand and the copy of the second strand to generate sequence reads, wherein a 5hmC in the first strand yields a mixture of T and C base calls and a 5mC in the first strand, in a position corresponding to the methylated CpG dinucleotide in the second strand, generates C base calls across the sequence reads.

In some embodiments, the fragment is from a sample from a subject and method may include mapping the sequence reads to reference data and independently identifying loci of 5hmC and 5mC in the subject. The sample may be from a tumor and the fragment may be tumor DNA. The identifying step may provide tumor-specific patterns of 5hmC and 5mC. Methods may include comparing the tumor-specific patterns of 5hmC and 5mC to a cancer database or atlas for the detection of evidence of cancer in the subject. Methods may include subsequently assaying a post-treatment sample from the subject for persistence of the patterns of 5hmC and 5mC as evidence of minimal residual disease.

In some embodiments, the sample comprises a FFPE slice of tumor tissue or a tumor biopsy. The post-treatment sample may be a liquid biopsy sample and the assaying step may involve analysis of cell-free DNA in the liquid biopsy sample. The methyltransferase may be, e.g., DNMT1 or DNMT5. Protecting modified cytosines may involve the use of a ten-eleven translocation (TET) enzyme and/or a beta-glucosyltransferase (β-GT). Converting the unmodified cytosine to the uracil may use apolipoprotein B mRNA editing enzyme, catalytic polypeptide-like 3A (APOBEC).

Aspects of the invention provide methods that include exposing a fragment to adaptors that include a linking adaptor and a bubble adaptor. For such embodiments, the linking adaptor contains a first oligo and a second oligo while the bubble adaptor contains a third oligo and fourth oligo (the numerals merely distinguish the oligos; if a bubble adaptor were used alone, it could be said to include a first oligo and a second oligo). In these embodiments, the linking adaptor includes a double-stranded portion in which a 3′ end of a first oligo is annealed to a 5′ end of a second oligo, wherein the first oligo is linked to a primer complementary to the second oligo. The bubble adaptor includes a third oligo partially annealed to a fourth oligo and the bubble adaptor further includes an unmatched segment in which a portion of the third oligo is not complementary to the fourth oligo. The methods include ligating the 3′ end of the first oligo to a first strand of the fragment, the 5′ end of the second oligo to a second strand of the fragment, a 5′ end of the third oligo to the first strand, and a 3′ end of the fourth oligo to the second strand. Methods further include extending the primer, annealed to the second oligo, to form an extension product that includes a copy of the second strand and the fourth oligo, thereby forming a complex for embodiments of mPS. The complex includes the first oligo ligated to the first strand and the third oligo, with the first oligo linked to the primer and the extension product with the second strand annealed to the extension product. Methods may include blocking 5mC and 5hmC from deamination; and methylating hemi-methylated in CpG dinucleotides in the extension product annealed to the second strand. In some embodiments, blocking the 5mC and 5hmC comprises converting 5mC to 5hmC (e.g., with a TET enzyme) and enzymatically glycosylating the 5mC and 5hmC (e.g., with BGT). Methods may include deaminating C in single-stranded DNA of the complex into U (e.g., using APOBEC).

Methods may include amplifying or copying the first strand and the copy of the second strand with a plurality of copies of a primer linked to a solid support to form a cluster. The solid support may be a bead and the amplifying may be performed in an aqueous compartment. The solid support may be a surface of a flow cell.

In some embodiments, methods include conducting a sequencing reaction with the first strand and the copy of the second strand to generate sequence reads, wherein 5hmC in the first strand appears as C across the sequence reads and 5mC in the first strands appears as a mixture of T and C in the sequence reads. Methods may include analyzing the sequence reads to independently identify patterns of 5hmC and 5mC in the fragment.

In certain embodiments, methods include annealing a universal primer to the portion of the third oligo not complementary to the fourth oligo and extending the universal primer so that the first stand is double stranded and the copy of the second strand is single stranded and linked to the first strand; performing a protection and conversion process so that C in single stranded DNA becomes U; and conducting a sequencing reaction on the first stand and the copy of the second strand to generate sequence reads, wherein 5hmC in the first strand appears as C and T among the sequence reads and 5mC in the first strand appears as C across the sequence reads.

### BRIEF DESCRIPTION OF THE DRAWINGS

FIG. 1 shows reagents and steps in a method.

FIG. 2 shows the use of two linked adapters.

FIG. 3 diagrams certain treatments useful to distinguish epigenetic modifications.

FIG. 4 diagrams a first version of methyl proximity sequencing (mPS v1).

FIG. 5 shows enzymatic reaction steps in mPS v1.

FIG. 6 shows copying linked fragments.

FIG. 7 shows the product of an amplification reaction.

FIG. 8 shows the sequencing of amplicons.

FIG. 9 is a table showing the bases and base modifications.

FIG. 10 is a table showing the bases and base modifications in mPS v1.

FIG. 11 shows bases and base modifications when mPS v1 uses TAPS.

FIG. 12 shows steps of a method for identifying modified bases.

FIG. 13 shows a second version of methyl Pro-Seq, mPS v2.

FIG. 14 shows additional steps in mPS v2.

FIG. 15 illustrates a first strategy for a sequencing reaction with a cluster.

FIG. 16 shows a second strategy with the cluster.

FIG. 17 shows bases and base calls in mPS v2, with DNMT, with EM-Seq reactions.

FIG. 18 shows bases and base calls of mPS v2 with DNMT and the EM-Seq.

FIG. 19 shows mPS v2 and TAPS.

FIG. 20 shows steps of a third version of methyl Pro-Seq, mPS v3.

FIG. 21 shows subsequent steps of mPS v3.

FIG. 22 shows the individual sequence reads.

FIG. 23 gives bases and base call of mPS v3 with EM-Seq.

FIG. 24 shows bases and base call of mPS v3 no DNMT.

FIG. 25 shows bases and base call when mPS v3 is used with TAPS.

FIG. 26 shows a fourth version of methylation proximity sequencing, mPS v4.

FIG. 27 shows steps of mPS v4.

FIG. 28 shows the base calls in mPS v4.

FIG. 29 is a table showing the bases and base calls involved in mPS v4.

FIG. 30 illustrates steps of a fifth version of methyl proximity sequencing, mPS v5.

FIG. 31 shows steps of mPS v5.

FIG. 32 shows first steps of a compartment workflow.

FIG. 33 shows additional steps of the compartment workflow.

FIG. 34 illustrates a sixth version of methyl proximity sequencing, mPS v6.

FIG. 35 is a table of bases and the base calls for mPS v6.

FIG. 36 is a table of bases and the base calls for mPS v6 with TAPS.

FIG. 37 illustrates error correction.

FIG. 38 shows two independently made copies from a fragment.

FIG. 39 shows a process for linking independent copies.

FIG. 40 shows a system.

FIG. 41 shows linked adaptors to make a complex useful in methods herein.

FIG. 42 shows a set of adaptors that may be used for mPS workflows.

FIG. 43 shows adaptors with various optional features.

FIG. 44 illustrates an mPS process using adaptors.

FIG. 45 shows the use of Y-adaptors in ‘6-base’ sequencing.

FIG. 46 shows a CAPS step and a resulting product.

FIG. 47 diagrams a genome sequencing process.

FIG. 48 shows steps in the mPS of Experiment 1.

FIG. 49 illustrates how base calls are made.

FIG. 50 gives a diagram of a workflow used in Example 1.

FIG. 51 shows sequence data from mPS t1.

FIG. 52 shows sequence data from mPS t2.

FIG. 53 shows sequence data from mPS t3.

FIG. 54 shows sequence data.

FIG. 55 is a diagram of the workflow.

FIG. 56 illustrates a Read 1 and Read 2 that may be generated.

FIG. 57 shows a workflow used in Example 2.

FIG. 58 is a plot (results) showing mC detection in template mPS t6.

FIG. 59 is a plot (results) showing hmC detection.

FIG. 60 gives results showing hemi-hmC detection.

FIG. 61 diagrams a workflow used in Example 3.

FIG. 62 shows the generation sequence reads.

FIG. 63 is a diagram of the workflow of Example 3.

FIG. 64 gives results showing mC detection.

FIG. 65 gives results showing hmC detection.

### DETAILED DESCRIPTION

Methods of the invention combine Pro-Seq with methylation library preparation methods to provide duplex error correction and methylation information in a single readout (mPS). Methods of the invention may be used to distinguish between 5mC and 5hmC. Methods of the invention have applications in early cancer detection, identifying a tumor-specific signature pattern of differential modification, detecting minimal residual disease, and other areas. Methods of the invention use proximity sequencing (Pro-Seq) with methods for detecting modified bases. Pro-Seq uses at least two linked molecules, each molecule being a strand of a fragment or a copy independently made from the fragment, in which the at least two linked molecules have essentially the same sequence of bases in the same orientation (i.e., are not reverse or complementary to each other with respect to the linkage). Those linked molecules form a complex and the complex is used as an input for a sequencing reaction, including any associated amplification reaction. An example of a complex that meets the requirement for proximity sequencing is a “forward” (or first or sense) strand of a DNA fragment that is linked to a copy of what was the reverse complementary “reverse” (or second or antisense) strand of that fragment, in which the forward strand and the copy of the reverse strand are linked by a linker via their 5′ ends. Where (i) chemical or enzymatic treatment of unmatched based modifications or (ii) polymerase error, DNA damage, sequencing error, or other phenomena imbue the strands with dissimilar bases at corresponding positions, copying by polymerase will create amplicons that do not match at that position. Sequencing from such a complex will generate sequence reads with a mixture of base calls for such a position. The mixture of base calls, and the knowledge of the chemical or enzymatic treatment of unmatched based modification, provide tools by which to report the presence of the base modifications in the original fragments.

Certain embodiments of mPS use a complex comprising a first (or “forward”) strand from a fragment linked to a copy of a second (or “reverse”) strand of the fragment. Chemical and/or enzymatic treatment of unmatched base modifications leaves dissimilar bases at corresponding positions in first strand and the copy of the second strand. The fragment used for mPS may be from any suitable biological sample including, for example, cell free DNA (cfDNA), a tissue sample, an FFPE sample, or others. Sample sources that include cfDNA may include blood, plasma, urine, lymph, cerebrospinal fluid (CSF), or any other suitable sample type.

As shown herein throughout, methods may include ligating adaptors to either or both ends of a fragment. The adaptors may include unique molecule identifiers (UMIs) or molecular index tags (MITs). UMIs or MITs may be provided as molecular barcodes within segments of the adaptors that are unique or nearly-unique across all of the adaptors used in an assay. In that context, members of a set of UMIs or MITs do not need to be truly unique but only need to be present with enough diversity that the chance of two duplicate UMIs or MITs being ligated to two identical fragments from a sample is vanishingly small (“nearly unique”) and such duplication ligation products are not present after ligation or are present in a vanishingly small number that does not affect quantitative measurements made using the unique or nearly unique barcodes. After UMIs are attached to fragments, after sequencing, sequence reads (comprising UMI sequence information plus template sequence information) may be de-duplicated (sometimes called “collapsing the sequence reads”), and each unduplicated sequence read represents one unique molecule in the sample. A count of unduplicated sequence reads that match one locus in a reference genome is a quantitative measure of a number of copies of that locus in the sample, and can thus be a measure of copy number changes, or copy number variation (CNV), in a sample.

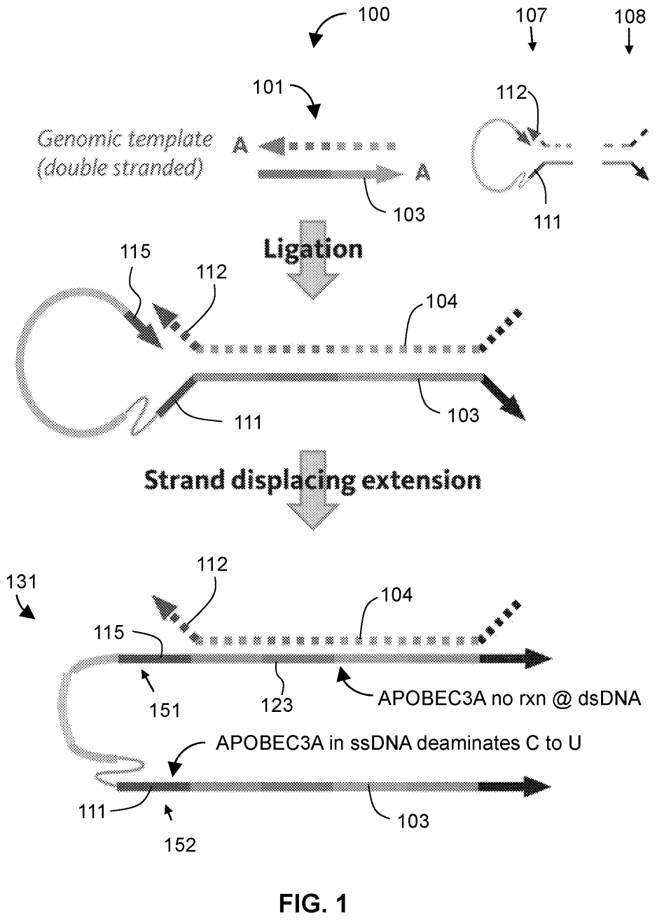

FIG. 1 shows reagents and steps in a method 100. The method 100 includes ligating a fragment 101 to a linking adaptor 107 in which a first oligo 111 is linked to a primer 115 annealed to a second oligo 112. By the ligation step, the first oligo 111 is ligated to a first strand 103 of the fragment 101. The method 100 includes extending the primer 115 to make an extension product 123 that includes a copy of a second strand 104 of the fragment 101.

This forms a molecular complex 131 that includes: the first strand 103 ligated to the first oligo 111, the first oligo 111 linked to the primer 115 wherein the primer 115 is extended into a product 123 that includes a copy of the second strand 104, and the second strand 104 annealed to the product 123 and ligated to the second oligo 112. The complex 131 may include primer binding sites 151, 152. Primer binding site 151 may be different from primer binding site 152 (or they may be the same as each other). The primer binding site 151 and the primer binding site 152 may have been introduced as part of the first oligo 111 and the second oligo 112 (in the linking adaptor 107). Primer binding sites such as the primer binding sites 151, 152 may be universal primer binding sites to which universal primers bind in a down-stream step and they may be bound to by sequencing primers. For example, in some embodiments, the complex 131 is used to seed a single sequencing reaction in which a first sequencing primer will bind to primer binding site 151 and a second sequence primer binds to site 152. Such embodiments are shown and discussed in greater detail herein below.

The linked fragments may optionally be protected against digestion, e.g. by RecJf & T7 exonucleases. That is any oligo of the adaptors may include modifications that render that oligo exonuclease resistant such that in downstream steps, certain extension products may be selectively digested away by exonuclease.

Prior to any amplification and sequencing reactions, the complex 131 is preferably subject to enzymatic conversion of modified bases and/or conversion of un-modified bases while modified bases are protected from conversion. In preferred embodiments, the method 100 includes converting a cytosine in the first strand 103 into a uracil.

As shown, the linking adaptor 107 performs the role of “linking” the first strand 103 for the fragment 101 to the extension product 123 as well as the annealed second strand 104 in the complex 131. A few features of the complex 131 are useful to downstream steps of preparing nucleic acids for sequencing. Notably, the first strand 103 and the extension product 123 include the same base sequence in the same 5′ to 3′ orientation. As shown, the first strand 103 and the extension product 123 are linked, by the linking adaptor 107, at their 5′ ends. The same base sequence appears in the same 5′ to 3′ orientation in the linked first strand 103 and extension product 123 and, notably, the first strand 103 is single-stranded while in the complex 131 while the extension product 123 is double-stranded, at this stage, in the complex 131.

At the depicted stage, it is optional for any portions of the complex to be protected against exonuclease. For example, for certain of the workflows shown in greater detail herein, the ends of the linked strands of the complex may include modified bases or phosphorothioate linkages allowing exonuclease to digest away second strand 104.

As discussed in greater detail in various embodiments herein, in a complex such as the complex 131, in a downstream step, a methyltransferase (e.g., DNMT1 or DNMT5) may be used to copy cytosine modification from the second strand 104 to the copy 123 of the second strand. An optional step before treatment with methyltransferase is to glycosylate any 5hmC in the complex so that 5mC but not 5hmC is copied to the copy 123 of the second strand.

As shown, the fragment 101 was linked to the linking adaptor 107 and also to a second adaptor 108. Other embodiments are within the scope of the disclosure.

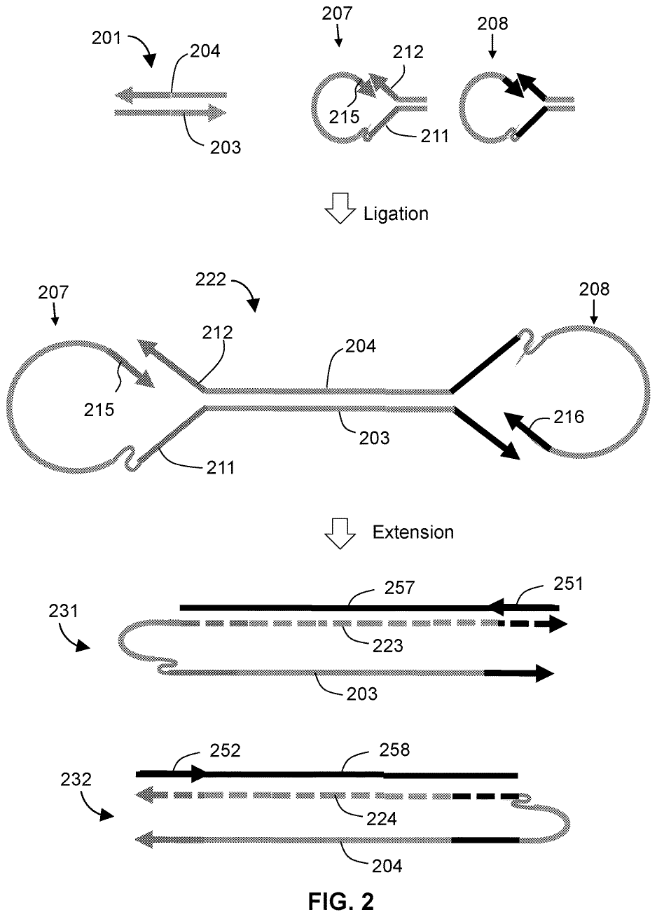

FIG. 2 shows the use of linked adapters on both sides. As shown, a fragment 201 is ligated to a first linking adaptor 207 and a second linking adaptor 208. Ligation forms an intermediate 222, in which a first oligo 211 of the first adaptor 207 is ligated to a first strand 203 of the fragment 201. The first oligo 211 is linked to a primer 215 annealed to a second oligo 212. Likewise, the second linking adaptor 208 is ligated and annealed at a distal end of the fragment 201, wherein a second primer 216 anneals a top oligo of the second linking adaptor 208 to a bottom oligo. The primers 216, 216 may be extended copy the two strands 203, 204 of the fragment 201. This produces a first stand complex 231 and a second strand complex 232. In the first strand complex 231, the first strand 203 is linked to a second strand copy 223 (which includes a copy of the second strand 204). The second strand complex 232 includes the second strand 204 linked to a first strand copy 224 (which includes a copy of the first strand 203). Primers 251, 252 that are designed to anneal to the single-stranded portions of the linking adaptors 207, 208 may be annealed to those portions of the complexes 231, 232 and extended to form primer extension products 257, 258. By those means, each of the complexes 231, 232 can include two polynucleotides with the same bases in the same order, linked at their 5′ ends, but in which of the two polynucleotides is single-stranded and other is double-stranded. Using two linked adaptors with a fragment 201 may include the use of any features such as the ligation adapters as shown or described in U.S. patent Ser. No. 11/879,151 B2, incorporated by reference.

Complexes prepared using methods of the invention may be exposed to enzymes that react differently on single-versus double-stranded polynucleotides.

For example, DNA methyltransferase (DNMT) enzymes such as DNMT1 or DNMT5 are enzymes that methylate C to 5mC when the C is present in double-stranded DNA, within a hemi-methylated CpG. In the complex 131, the copy of the second strand is “new” and any Cis initially un-modified. But if the second strand includes a 5mC in a CpG dinucleotide, then the DNMT enzyme will act on that double-stranded portion to methylate the “new” C into 5mC.

Additionally, there are enzymes that can be used to protect bases. In a downstream step, certain bases may be enzymatically de-aminated to convert C to U or to convert 5mC to T. However, a ten-eleven translocation (TET) enzyme can convert 5mC to 5-hydroxy-methylcytosine (5hmC) and beta-glucosyltransferase (β-GT) can glycosylate 5hmC into β-glucosyl-5-hydroxymethylcytosine (5gmC), which protects 5mC from downstream enzymatic de-amination.

Downstream enzymatic de-amination may be performed by Apolipoprotein B mRNA editing enzyme, catalytic polypeptide-like 3A (APOBEC), which can convert C to U and 5mC to T. Notably, APOBEC de-aminates cytosine, only in single-stranded DNA, converting that cytosine to uracil. APOBEC is one exemplary enzyme that deaminates cytosine only in single-stranded DNA, converting C to U or 5mC to T.

By those mechanisms, the complex 131 may be treated with a DNMT enzyme to methylate any C in the double-stranded portion that is hemi-methylated in a CpG dinucleotide. At that stage, a corresponding C in the first strand will not be methylated because the first strand is single-stranded. The use of a TET enzyme can convert 5mC to 5-hydroxy-methylcytosine (5hmC) and beta-glucosyltransferase (β-GT) can glycosylate any 5hmC, forming 5gmC. Any C in the first strand what was unmodified in the fragment 101 will remain as an un-modified, un-protected C. The complex 131 may then be treated with APOBEC to convert that un-modified, un-protected C into a uracil (U).

To summarize, a cytosine that was present and un-modified in the first strand 103 will be converted to U when the complex 131 is treated with APOBEC. The corresponding cytosine in the copy 123 of the second strand will remain a C because APOBEC acts on single stranded DNA. A methylated cytosine in a CpG in the first strand 103 will appear as C in the copy of the second strand that gets methylated to 5mC by the DNMT enzyme. The methylated cytosine in a CpG in the first strand 103 will be converted to T by APOBEC. A methylated C in the first strand will not be converted if it has been protected by TET.

Methods may include amplifying the first strand and the copy of the second strand using a plurality of primers linked to a solid support (e.g., surface of a flow cell or bead) to form a cluster on the solid support. In some embodiments, the solid support is a bead and the amplification is performed in an aqueous compartment such as a well or droplet.

If the copy 123 of the second strand and the first strand 103 are copied with polymerase (e.g., amplified), the polymerase will incorporate a G into amplicons where it encounters the 5mC and the polymerase will incorporate A where it encounters U or T. That copying may be performed by capturing the complex 131 with linked primers that are linked together or to a solid support (e.g., flow cell or bead) in which the primers anneal to the copy 123 of the second strand and the first strand 103. By that mechanism, the complex 131 seeds an amplification reaction that results in a cluster e.g., linked to the solid support.

Methods may include conducting a sequencing reaction on the cluster to generate sequence reads, wherein the sequence reads contain (i) a mixture of T and C base-calls at a position corresponding a methylated cytosine, and (ii) C base calls for cytosine. Depending on process steps used to treat the complex, the sequence reads will include different combinations of base calls for certain modified bases in the original fragment.

Sequencing those amplicons will produce sequence reads with a mixture of G and T base-calls at that position. Because the copy of the second strand and the first strand are linked at their 5′ ends and have same base sequencing (but for any epigenetic modifications, deaminated bases, DNA damage, polymerase errors, sequencing errors, etc.), the linked first strand and copy of the second strand may be used as the input to amplification reaction that will progress as if the amplification reaction were initiated with a single, single-stranded molecule, except that the amplicons from that amplification reaction will have a mixture of bases at a position in which the linked first strand and copy of the second strand do not match each other due to treatments (e.g., enzymatic or chemical) described herein.

Methods of the invention encompass various approaches to sequencing strands that are linked together, are made from one starting fragment, and have base sequences that are substantially identical except for treatments to distinguish epigenetic modifications from each other and from unmodified bases.

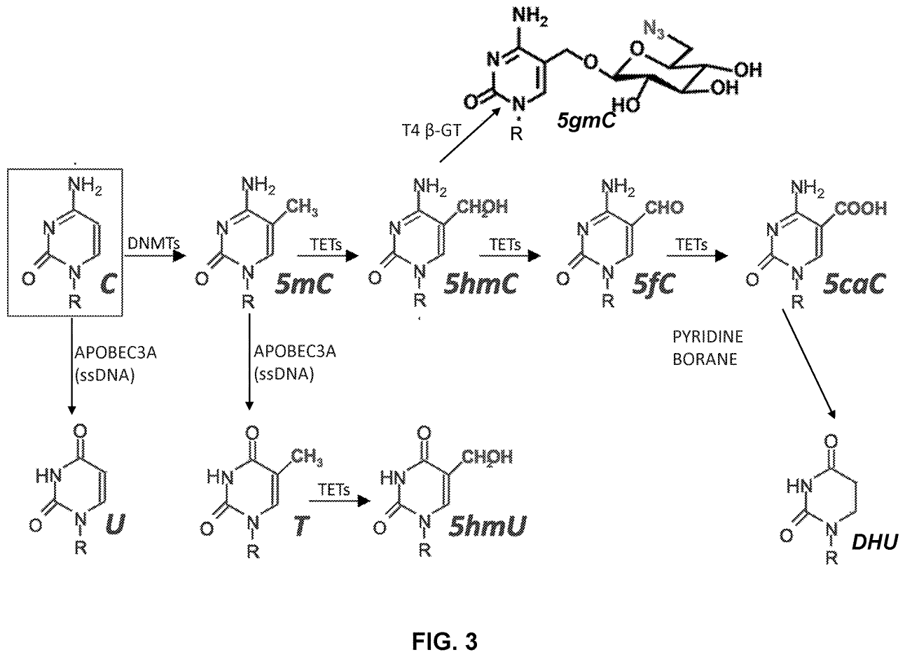

FIG. 3 diagrams certain treatments useful to distinguish epigenetic modifications from each other and from unmodified bases. Using cytosine (C) as a starting point, it can be seen that DNMT enzymes methylate C to 5mC. When C or 5mC are present in single-stranded DNA, APOBEC deaminates those bases to U or T, respectively. However, a 5mC in the original DNA may be protected against deamination by TET enzymes (and optionally β-GT). TET enzymes convert 5mC to 5hmC, 5fC, and 5caC, and β-GT turns 5hmC to 5gmC. APOBEC does not deaminate 5gmC, 5fC, or 5caC. That is, by virtue of the TET enzymes, 5mC is protected against deamination. When those bases are read by DNA sequencing, U is read as T, while 5gmC, 5fC, or 5caC are read as C. Thus TET protection and deamination contribute to sequencing results that distinguish between C and 5mC in the original strand.

It is noted that APOBEC reacts with single-stranded DNA (ssDNA) to deaminates C to T or 5mC to U, while APOBEC does not catalyze those reactions in double-stranded DNA (dsDNA). TET enzymes (“TETs”) such as TET1 convert 5mC to 5hmC. Phage T4 β-GT (T4-β-GT or β-GT) glycosylates 5hmC to 5gmC. TET enzymes convert 5hmC to 5-formylcytosine (5fC), and convert 5fC to 5-carboxylcytosine (5caC). It is noted that 5caC may be chemically converted using pyridine borane to dihydrouridine (DHU). Finally, TET enzymes convert T to 5 hydroxymethyl uridine (5hmU). It is additionally noted (but not shown here) that DNA methyltransferases DNMT1 and DNMT5 react with hemi-methylated CpG dinucleotides in dsDNA to methylate the unmethylated C to 5mC.

Methods of the invention are explained herein making reference to some of the reactions and the pathways shown here in combinations that provide for identifying patterns of modified bases when using proximity sequencing (Pro-Seq) library preparation. The reactions and pathways that are shown may be combined to form one of the methylation-specific workflows known as EM-Seq, E5hmC-Seq, TAPS, TAPSB, CAPS, bisulfite sequencing (not shown in the figure), TAB-Seq, and oxBS-Seq, among others. When combining one of those workflows with Pro-Seq library preparation, one may optionally also include (or not include) an optional step of protection with DNMT (labeled “+/−DNMT” or “optional protection and methyltransferase” in some diagrams). For example, some embodiments of Pro-Seq use a complex that includes a forward strand linked to a copy of a reverse strand with the reverse strand annealed to the copy of the reverse strand. It is optional to treat the complex with DNMT, which will fully methylate any hemi-methylated CpG dinucleotides within the dsDNA portion (the reverse strand annealed to the copy of the reverse strand).

It is noted that the adaptor ligation methods here preserve the ends of the fragments and can be used to show genomic and epigenomic alterations in the ends of the fragments. Thus methods of the invention provide for fragmentomics-based analysis of samples. Fragmentomics may involve the detection of size patterns of fragments in a sample and may be used to determine tissue origin or to identify tumor-DNA, e.g., circulating tumor DNA. See Theirry, Circulating DNA fragmentomics and cancer screening, Cell Genomics, 3:100242, incorporated by reference. Analysis of sequence reads generated by methods of the invention and their counts, and mapping, and identified loci and patterns of differential modifications therein, are useful as inputs to machine learning platforms for the detection of cancer and minimal residual disease such as detection platform MRD-EDGE. See Widman, 2024, Ultrasensitive plasma-based monitoring of tumor burden using machine-learning-guided signal enrichment, Nat med 30 (6): 1655-166, incorporated by reference. Such applications and outcomes are produced using workflows for detection of base modification such as EM-Seq, E5hmC-Seq, Five-letter seq, Six-letter seq, TAPS, TAPSB, CAPS, bisulfite sequencing, TAB-Seq, oxBS-Seq, EnIGMA, DM-Seq, Methyl-CODEC, among others. Each of those workflows, any step or component of any of those workflows, and combination of those workflow or steps or components of those workflows may be used together in any suitable combination in methods of the invention.

Some of those workflows are briefly summarized here.

### EM-Seq

Enzymatic methyl-seq (EM-seq) is used to detect 5mC and 5hmC using two sets of enzymatic reactions. In the first reaction, TET2 and T4-BGT convert 5mC and 5hmC into 5gmC, products that cannot be deaminated by APOBEC. In the second reaction, APOBEC deaminates unmodified cytosines by converting them to uracils. APOBEC reacts with single-stranded DNA. See Vaisvila, 2021, Enzymatic methyl sequencing detects DNA methylation at single-base resolution from picograms of DNA, Genome Res 31:1280, incorporation by reference.

### E5hmC-Seq

Enzymatic 5hmC-seq (E5hmC-seq) provides for the specific detection of 5hmC. In a first step of E5hmC-Seq, 5hmCs are glycosylated, which protects them from subsequent deamination by APOBEC. Glycosylation of 5hmC in genomic DNA does not inhibit replication by DNA polymerase and amplification products will incorporate a C in the corresponding position. In a second step of E5hmC-Seq, cytosines and 5mCs are deaminated resulting in their conversion to uracil and thymine, respectively. When replicated, amplification products will incorporate T in the corresponding position. Sequencing those products allows discrimination of 5hmC from cytosine and 5mC in the starting template. See Cammarata, 2024, Abstract 7008: NEBNext E5hmC-seqTM: Direct detection of 5-hydroxymethylcytosine at single base resolution, Cancer Res 86 (6 Suppl): 7008, incorporated by reference.

### Five-Letter Seq

Five-letter seq is a workflow in which a first strand is linked to a copy of a the first strand. The copy of the first strand, being newly synthesized, will include an unmodified C at any position in which the first stand includes a modified C. Modified Cs are protected using a TET enzyme and BGT. Treatment by APOBEC deaminates unprotected Cs are to U (read as T). The strands are amplified and sequenced. Base calls across the sequence reads are resolved into one of five states: A, C, modC, G, T. Five-letter seq is discussed in Fullgrabe, 2023, Simultaneous sequencing of genetic and epigenetic bases in DNA, Nat Biotech 41:1457-1464; US 2022/0298551 A1; and US 2022/0290215 A1, the contents of which are incorporated by reference.

### Six-Letter Seq

Six-letter seq is a workflow in which a first strand is linked to a copy of a the first strand. The copy of the first strand, being newly synthesized, will include an unmodified C at any position in which the first stand includes a modified C. The copy of the first strand is preferably annealed to the first strand. DNMT5 copies 5mc (but not 5hmC) from the first strand to the copy of the first strand. After the DNMT, modified Cs are protected by a TET enzyme and BGT and then unprotected bases are deaminated with APOBEC. The strands are amplified and sequenced. Base calls across the sequence reads are resolved into one of six states: A, C, 5mC, 5hmC, G, T. Six-letter seq is discussed in Fullgrabe, 2023, Simultaneous sequencing of genetic and epigenetic bases in DNA, Nat Biotech 41:1457-1464; US 2022/0298551 A1; and US 2022/0290215 A1, the contents of which are incorporated by reference.

### TAPS

TET-assisted pyridine borane sequencing (TAPS) combines an enzymatic step followed by a chemical reaction to detect 5mC and 5hmC TAPS relies on the ability of tet methylcytosine dioxygenase 1 (TET1) to oxidize all the 5mC and 5hmC into 5-carboxycytosine (5caC). The 5caCs are then reduced to dihydrouracil (DHU) using pyridine borane. PCR then converts DHU to thymine, enabling cytosines and the corresponding cytosine modifications to be differentiated. See Liu 2019, Bisulfite-free direct detection of 5-methyl cytosine and 5-hydroxy methylcytosine at base resolution, Nat Biotech 37:424-429, incorporated by reference.

### TAPSβ

TAPS with β-glucosyltransferase (βGT) blocking (TAPSβ) first uses βGT to block 5hmC (as 5gmC). Then, TET converts 5mC to 5caC, after which pyridine borane converts the 5caC to DHU, which PCR incorporates as T, while the PCR incorporates the blocked 5gmC as C. See Liu, 2021, Subtraction-free and bisulfite-free specific sequencing of 5-methylcytosine and its oxidized derivatives at base resolution, Nat Comm 12:a618, incorporated by reference.

### CAPS

Chemical-assisted pyridine borane sequencing (CAPS) uses a chemical such as potassium perruthenate (KRu04) to oxidize 5hmC to 5fC, after which 2-methylpyridine borane (pic-borane) or a related agent chemical converts 5hmC to DHU, which PCR copies as T. See Liu, 2021, Nat Commun 12:618.

### TAB-Seq,

Tet-assisted bisulfite sequencing (TAB-seq) is a method useful to identify 5-hmC at single-base resolution, as well as determine its abundance at each modification site. TAB-Seq involves β-glucosyltransferase (β-GT)-mediated protection of 5-hmC (glycosylation) and using a TET enzyme to convert 5-methylcytosine (5-mC) to 5-carboxylcytosine (5-caC). After the subsequent bisulfite treatment and PCR amplification, both cytosine and 5-caC (derived from 5-mC) are converted to thymine (T), whereas 5-hmC reads as C.

oxBS-Seq

Oxidative bisulfite sequencing (oxBS-seq) can quantitatively locate 5mC and 5hmC at single-base resolution in genomic DNA. In bisulfite sequencing (BS-seq), both 5mC and 5hmC are read as cytosines and thus cannot be discriminated; however, in oxBS-seq, specific oxidation of 5hmC to 5-formylcytosine (5fC) and conversion of the newly formed 5fC to uracil (under bisulfite conditions) means that 5hmC can be discriminated from 5mC. A positive readout of actual 5mC is gained from a single oxBS-seq run, and 5hmC levels are inferred by comparison with a BS-seq run. Booth, 2013, Oxidative bisulfite sequencing of 5-methylcytosine and 5-hydroxymethylcytosine, Nat Protoc 8 (10): 1841-1851, incorporated by reference.

### EnIGMA

The Enzyme-assisted Identification of Genome Modification Assay (EnIGMA) provides method for the direct identification of mC, hmC and unmodified cytosine (C) at a single base resolution. In EnIGMA, after adaptor ligation, cleavage, and denaturing, each strand is copied by polymerase to synthesize its complementary strand, creating two dsDNA products. The dsDNA products are treated with DNMT1, and then bisulfite, followed by PCR and sequencing. The sense and antisense strands of the original fragment and the respective two synthesized copies provide four sequence read-outs in which the combination of C and T base calls inform of 5mC, 5hmC, and C in the original. See Kawasaki, 2017, A Novel method for the simultaneous identification of methylcytosine and hydroxymethylcytosine at a single base resolution, Nucleic Acids Res 45(4):e24, incorporated by reference.

### DM-Seq

Direct methylation sequencing (DM-Seq) is a bisulfite-free method for profiling 5mC at single-base resolution using nanogram quantities of DNA. DM-Seq employs two key DNA-modifying enzymes: a DNA methyltransferase and a DNA deaminase capable of precise discrimination between cytosine modification states. In the final DM-Seq workflow, 5-propynylcytosine (5pyC) adapters are ligated to sheared gDNA and copied using 5mCTP to create a strand exclusively containing 5mCs in place of C. The gDNA is then protected by CpG-specific carboxymethyltransferase (CxMTase) acting on unmodified CpGs to form 5-carboxymethylcytosine (5cxmC) and glycosylation by BGT (for 5hmCs). Subsequent deamination by A3A is performed before PCR amplification and sequencing. Wang, 2023, Direct enzymatic sequencing of 5-methylcytosine at single-base resolution, Nat Chem Biol 19(8):1004-1012, incorporated by reference.

### Methyl-CODEC

Methyl-CODEC simultaneously uses methylation sequencing and duplex sequencing using single read pairs. In Methyl-CODEC, an enzymatically methyl-converted sense strand is linked with a copied antisense strand of the native sequence, and vice versa, to provide duplex sequencing accuracy and single-base methylation resolution. Specifically, an enzymatically deaminated sense strand is linked to the reverse complement of the antisense strand, which is protected from conversion by using conversion-resistant dCTPs in a strand linking step. Methyl-CODEC is described in Liu, 2024, Methyl-CODEC enables simultaneous methylation and duplex sequencing, bioRxiv 2024.12.23.630102, incorporated by reference.

Of those workflows, methods of the invention may use any one or may use more than one in combination. For example, using the complex of Pro-Seq, some embodiments use EM-Seq first (only works on ssDNA), then TAPS. As is shown herein, after sequencing the treated Pro-Seq complex, sequence reads may include a mixture of base-calls as the product of differential treatment by the employed process steps at a position within the strands of the complex. The identify and modification state of a base in the fragment may be determined by the base-calls in the sequence reads. In some embodiments, sequential information may be used to interpret data. For example, where a modified C must be followed by G due to patterns of modification in CpG dinucleotides. A system of the invention may interpret the base calls using such logic programmed in. For example, where the base calls indicate a 5C that is not followed by a G, systems of the invention may discard the call of a methylated base. Stated more generally, an analysis unit of the invention may be programmed to “discard” methyl calls in CHH or CHG regions (using the IUPAC codes).

Additionally, enrichment (such as a hybrid capture pull down) could be performed after linked fragment generation, i.e., formation of the Pro-Seq complex. Further, some embodiments of the disclosure make use of a linked-target capture to link together two fragments such as the sense strand and the anti-sense strand, or corresponding copies thereof, from a starting sample.

In various embodiments of the disclosure, adaptors may be conversion resistant, e.g., may contain no cytosines or may use 5mC instead of C.

Various specific embodiments of and approaches to the methyl-Pro-Seq (mPS) workflow are shown and discussed herein.

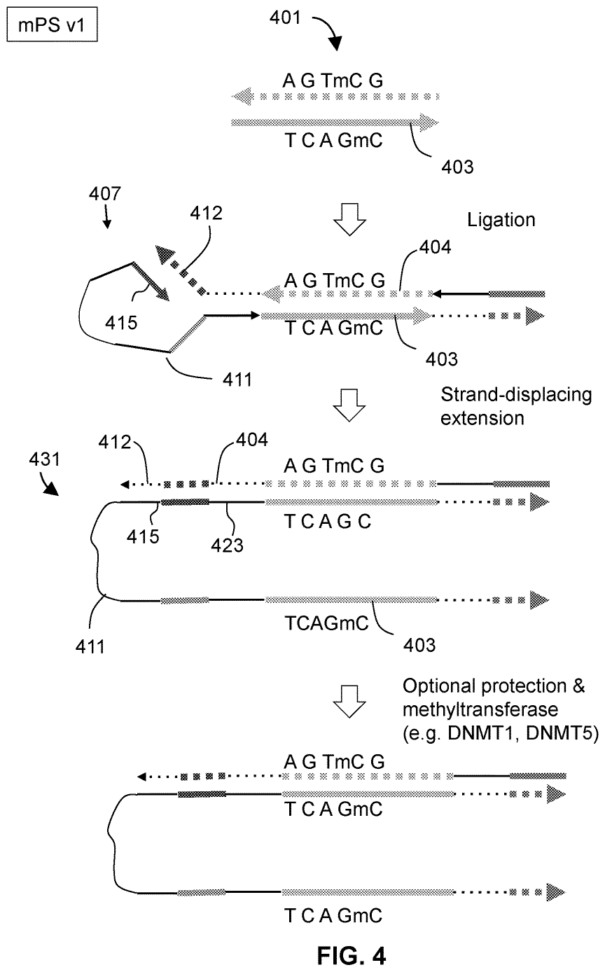

FIG. 4 diagrams a first set of reactions that may be used in embodiments of a first version of mPS (mPS v1). As shown, a fragment 401 is ligated to a linking adaptor 407 in which a first oligo 411 is linked to a primer 415 annealed to a second oligo 412. By the ligation, the first oligo 411 is ligated to a first strand 403 of the fragment.

The primer 415 is extended to make a copy 423 of a second strand 404 of the fragment 401. This produces a molecular complex 431 that includes: the first strand 403 ligated to the first oligo 411, the first oligo 411 linked to the primer 415 wherein the primer is extended into a product that includes a copy 423 of the second strand, and the second strand 404 annealed to the product and ligated to the second oligo 412.

This first version of methyl proximity sequencing (mPS v1) may include an optional protection step in which 5hmC is glycosylated by an enzyme such as BGT after which a methyltransferase such as DNMT5 would copy 5mC from the second strand 404 to the copy 423 of the second strand, but not copy the 5hmC.

This first version of methyl proximity sequencing (mPS v1) may optionally include treating the complex 431 with a DNMT enzyme (e.g., DNMT1 or DNMT5) to methylate a C in the copy 423 of the second strand 404. If 5hmC were not protected, DNMT5 may copy the hydroxy-methyl modification from the second strand 404 to the copy 423 of the second strand. The mPS v1 embodiment may proceed to steps similar to those of an EM-Seq treatment.

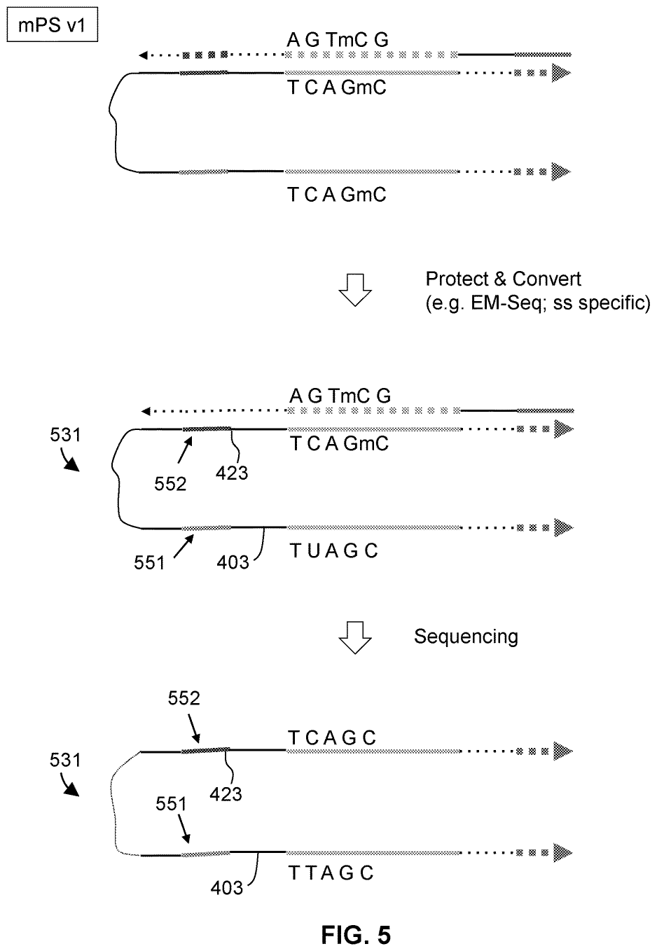

FIG. 5 shows further steps in mPS v1, specifically, enzymatic reaction steps from EM-Seq being performed on the complex. Enzymes such as TET and beta-GT may be used to protect modified cytosines in the complex 431, after which APOBEC is used to convert, in a single-strand specific manner, a first cytosine in the first strand into a uracil. APOBEC is an enzyme that only acts on bases in single-stranded DNA. As shown, C in the dsDNA portion remains C after the “converting” step. As shown, the C in the dsDNA portion is a cytosine in the copy of the second strand. That cytosine is matched, in position (a “matched cytosine”) to the position of the converted C, now U, in the first strand. That is, as shown, the copy 423 of the second strand includes a matched cytosine at a position corresponding to a position of the first cytosine, which is now a uracil, in the first strand 403.

This construct may now be referred to as a post-conversion complex 531. The post-conversion complex 531 may include primer binding sites 551, 552. Primer binding site 551 may be different from primer binding site 552. The primer binding site 551 and the primer binding site 552 may have been introduced as part of the first oligo 411 and the second oligo 412 (in the linking adaptor 407). The post-conversion complex 531 may be used to seed a single sequencing reaction (shown in more detail below), at which stage a first sequencing primer may anneal to primer binding site 551. The sequencing reaction may include annealing a second sequencing primer to the second primer binding site 552.

The depicted workflows are not strictly EM-seq because the complex 431 being treated includes two dissimilar segments that may both be treated simultaneously, all the way through sequencing. The copy 423 of the second strand and the first strand 403 begin with the same bases oriented through the linkage “in phase” (in same 5′ to 3′ orientation). However, due to differential treatment of the two strands, the base composition may not be perfectly matched when the complex 431 is subject to amplification and sequencing. As shown, the post conversion complex 531 (before the “Sequencing” step) has a U in the second labeled position of the first strand 403 while the matched second labeled position of the copy 423 of the second strand carries a C. When amplified and sequenced, the U in the first strand 403 of the post-conversion complex 531 will be read as a T.

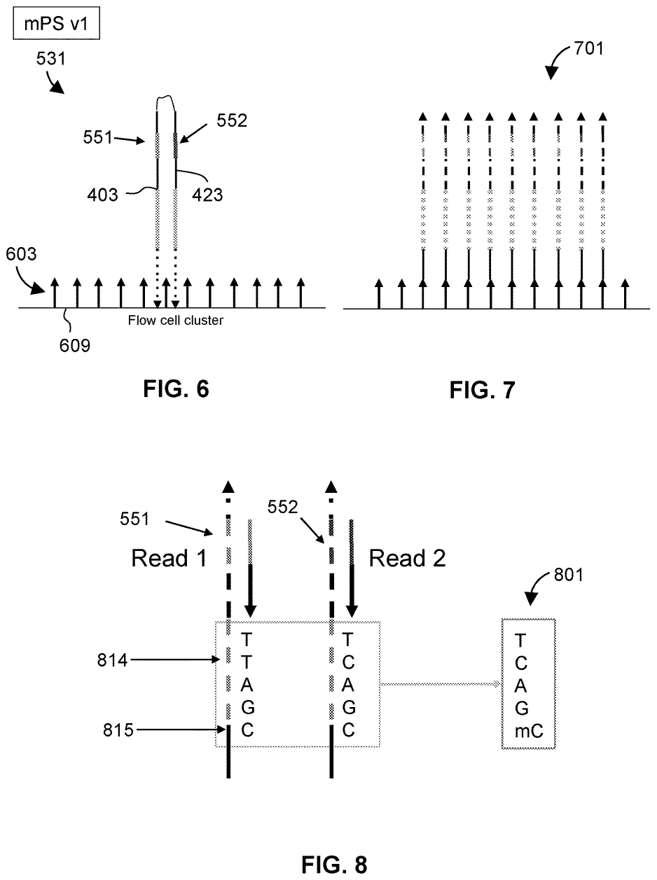

FIG. 6 shows a step of one method 600 for copying linked fragments using primers linked via a solid support. A post conversion complex 531 is captured by primers 603 that are linked to each other, optionally through a linker 609. The linker 609 may include PEG, a dendrimer, a bead, a hydrogel, or a solid support such as the surface of a flow cell. Because the first strand 403 and the copy 423 have substantially the same sequence in the same 5′ to 3′ orientation (but for chemical or enzymatic changes to modified bases), the primer binding ends of those strands (e.g., provided by ligated adaptors) are the same among those strands, and the primer 603 may all be clonal copies of one primer sequence.

The method 600 includes coping the copy 423 of the second strand and the first strand 403 with polymerase. A plurality of clonal copies of a primer 603 (e.g. complementary to a primer binding site in the adaptor) are attached together by a linker 609, such as a solid support such as a surface of a flow cell (as shown) or a bead. The post conversion complex 531 is annealed to those primers.

FIG. 7 shows the product of the polymerase copying those strands in an amplification reaction. The polymerase will base-pair an A with the U and base-pair a G with the C. If those products are amplified with polymerase to produce amplicons, those amplicons will include a mixture of T and C at that position. Each strand of the complex may be copied multiple times, or the amplification may use reverse primers also bound to the surface (e.g., for bridge amplification) or reverse primers in solution, or a combination of those approaches. The product of amplification is a cluster 701. The cluster may be linked to a bead or a flow cell. A sequencing reaction may be conducted on the cluster.

FIG. 8 shows the binding of two sequencing primers to amplicons of the cluster, which are extended to generate read 1 and read 2 of a plurality of sequence reads. For the single sequencing reaction, a first sequencing primer may anneal to primer binding site 551 to generate Read 1. The sequencing reaction may include annealing a second sequencing primer to the second primer binding site 552 to generate Read 2. In preferred embodiments, Read 1 is performed across all clusters simultaneously, and then read 2 is performed simultaneously. As drawn, Read 1 will be a read from the copies of the first strand represented in each cluster and Read 2 will be a read from the copies of the copy of the second strand represented in each cluster, or vice versa. Note that the post-conversion complex 531 includes the primer binding sites 551, 552, which were provided by the linking adaptor.

Different sequencing platforms may provide the reads as output in different fashions. For example, the sequencing by synthesis instruments sold under the trademark ILLUMINA will output multiple individual reads, e.g., in a FASTQ or FASTA file. The individual reads from an ULTIMA instrument may have a mixture of signals (e.g., a mixture of T and C signals at corresponding positions in a read) that indicate the identity of the base in the original input molecule (e.g., C).

The base calls of those sequence reads are drawn alongside the amplicons. As can be seen, position 814 of read 1 will include a base call of T. Position 814 of read 2 will include a base call of C. Position 815 of read 1 will include a base call of C. Position 815 of read 2 will include a base call of C. To the right of the amplicons is drawn a box showing the final base calls 801 that are made for the fragment 401 based on the base calls among the individual sequence reads.

Due to the fact that the fifth labeled position in the first strand 403 was methylated cytosine, conversion with APOBEC had no effect on that position within the complex 431. This means that all individual sequence reads have a base call of C at position 815, and an analysis unit of a connected system can make a base call of 5mC for position 815 of the first strand 403 of the fragment 401. Due to the fact that position 814 in the first strand 403 was cytosine, conversion with APOBEC left a U in the complex. As a result, the sequence reads show a mixture of T and C base calls at position 814. The analysis unit can make a base call of C for position 814 of the first strand 403. Other sequencing platforms such as the sequencing instruments sold under the trademark ULTIMA may by default output a single read for a cluster, in which the mixture of signals is resolved within the base calling logic of the instrument or an analysis unit. In an Ultima instrument, a cluster is present as a plurality of bead-linked amplicons with the bead held in positions electrostatically over a (circular) flow cell. The instrument sequences the cluster by introducing one base at time in which a portion of the bases are fluorescently labeled and optically detecting incorporated bases. For example, for the cluster shown, the apparent mixture of T and C bases may be optically detected within an ULTIMA instrument as two colors of fluorescence and the instrument or analysis unit may be programmed to “call” the corresponding position as C. In this example, a position 815 in the cluster 701 that is occupied across all (or substantially all) strands by C, will give different “sequence reads” in different instruments (e.g., ILLUMINA versus ULTIMA). From an ILLUMINA instrument, the large number of sequence reads (e.g., within a FASTQ file) will all have C at that position 815 (some small number of the reads may have a different base call due to, e.g., sequencing error, and Pro-Seq provides a mechanism to correct for such sequencing error). From an ULTIMA instrument, the optical signal from position 815 will include substantially only one color of fluorescence at position 815, the instrument will output a sequence read indicating a 5mC at position 815. At a position 814, the cluster 701 may include a mixture of T and C bases. Output from an ILLUMINA sequencer may be a plurality of (e.g., millions of) sequence reads with a mixture of T and C base calls for position 814. Output from an ULTIMA instrument may be one sequence read (interpreted from a mixture of two colors of fluorescence from position 814) carrying a base call of C at position 814.

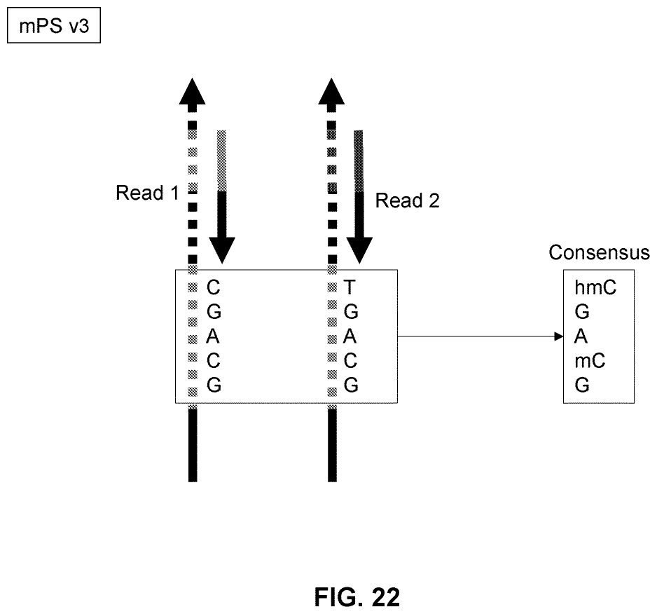

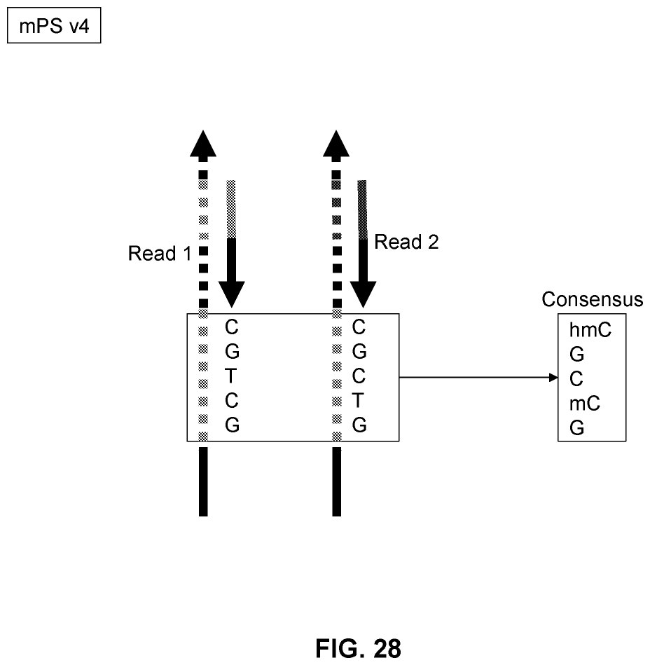

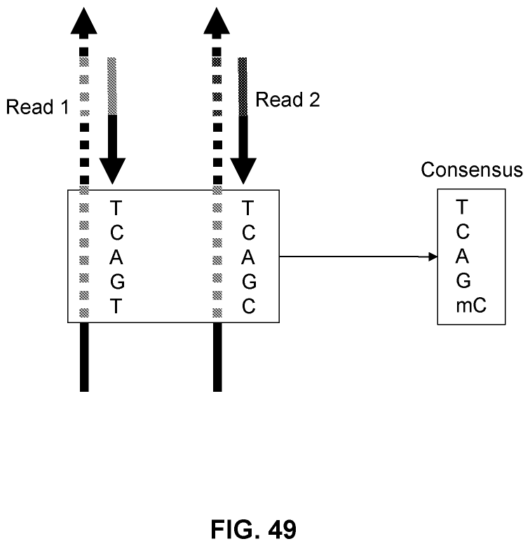

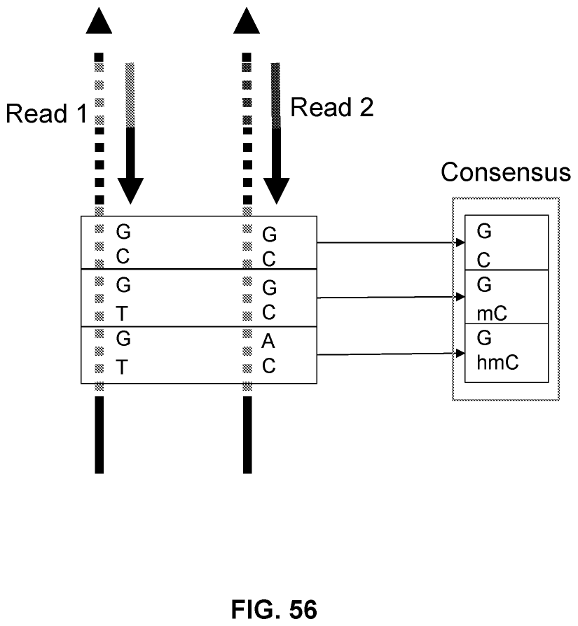

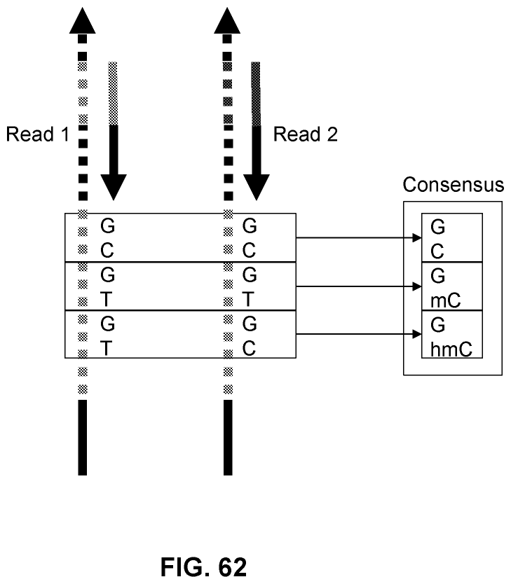

Considering the consensus base calls diagrammed in FIG. 8, which are made base on instrument base calls in Read 1 and Read 2 as shown, it is important to note that sequencing reactions to generate a sequence read, the sequence reads, and making a consensus base call (with identities and positions of A, C, G, T, 5mC, and 5hmC as well as lesions, polymerase errors, and sequencing errors) may follow one of a few variant embodiments. This note applies to FIG. 8, FIG. 16, FIG. 22, FIG. 28, FIG. 49, FIG. 56, and FIG. 62 plus the various tables of strand or sequencing primer-specific base calls with consensus or fragment base calls (here, “base call” includes epigenetic features and false variants being lesions or errors). The approaches are drawn as if two sequence reads are obtained from one cluster, e.g., in an ILLUMINA sequencer. That is a (i) first consensus calling embodiment and in that first consensus calling embodiment, one Pro-Seq complex is used to seed one cluster on an ILLUMINA flow cell. Each input strand of the complex is given its own sequencing primer binding site by virtue of the adaptors used in library preparation. Two sequencing reactions are conducted (e.g., one after the other). The first reaction uses first read primer to generate read 1 and the second uses a second read primer to generate read 2, creating results as shown in e.g., FIG. 8. In a (ii) second consensus calling embodiment, the same data may be collected with a single sequencing reaction on an ILLUMINA instrument. In the second consensus calling embodiment, mismatched bases at homologous positions within a cluster (e.g., a position from a sense strand and a corresponding position from the antisense strand) may promote incorporation of differently labeled bases. E.g., where a C has become U and is read as a T but a homologous mC in the other strand is read as a C, the ILLUMINA instrument may (in one sequence reaction) incorporate two different colors of fluorescently labeled bases. Software can read the signals from the raw ILLUMINA data and detect the mismatched bases at the homologous positions. This second consensus calling embodiment may use custom designed base-calling software that accesses ILLUMINA's “under the hood” raw read data. In a (iii) third consensus calling embodiment, mismatched homologous positions are read using a 9QAM or 16QAM encoding scheme. A 9QAM encoding scheme can be used to accurately differentiate between two simultaneously received base calls. Plotting relative intensities of light signals obtained from 2 reads generates a constellation of 9 clouds (16QAM is similar but with 4×4, or 16 clouds). The four corner clouds represent high quality and accurate base calls, while off-corner clouds represent potential library prep or sequencing errors, which could be eliminated. As applied here, 9QAM may simultaneously sequence genomic and epigenetic data using the mPS and/or hmPS workflows herein. In a first instance, the four corner clouds of 9QAM may be A, C, G, and T. Herein in the third consensus calling embodiment, any of the corner clouds, or any of the 9 clouds, may be allocated to modified bases. 9QAM or 16QAM base-calling is discussed generally in WO 2023175021 A1 and WO 2023175013 A1, and for methyl-sequencing in WO 2026006746 A2, the contents of each of which are incorporated by reference. In a (iv) fourth consensus calling embodiment, positions in a cluster are read in color space (as compared to base space) so if a cluster includes mismatched bases at homologous positions on strands, during a sequencing reaction, a mixture of different bases (which may be labeled with different colored fluorophores) may be incorporated. Sequencing instruments may detect the mixture of colors and software program logic in the sequencing instrument may “call” a position based on logic in FIG. 8, FIG. 16, FIG. 22, FIG. 28, FIG. 49, FIG. 56, and FIG. 62 plus the various tables of strand or sequencing primer-specific base calls with consensus or fragment base calls as shown herein. To determine base calls in color space, an mPS complex prepared, e.g., with a linking primer and converted using workflows herein may be sequenced on an ULTIMA genomics sequencing instrument. In a (v) fifth consensus calling embodiment, after an mPS complex is formed and treated according to workflows herein, it is prepared for single-molecule sequencing. For example, two strands representing sense and antisense strands of one fragment may be converted and, e.g., circularized or concatemerized so that independent copies of the two strands actually appear in tandem in one long molecule. The long molecules may be subject to single-molecule sequencing using a platform from Oxford Nanopore Technologies or Pacific Biosciences. Read information for the independent copies may be parsed out of the resulting sequence data based on position or any index or barcode and may be aligned to each other and used for consensus calling as otherwise shown herein. In any event, consensus calling may proceed according to any suitable embodiment and preferred embodiments may include two or more independent reads on an ILLUMINA instrument, i.e., the (i) first consensus calling embodiment, or a single read in color space on an ULTIMA instrument, i.e., the (iv) fourth consensus calling embodiment.

With systems of the invention, the sequencing instrument or a connected analysis unit may include computer program logic that makes final base calls 801 for the original fragment based on the instrument signals and readings. The set of final base calls 801 is an important output of methods of the invention. The set of final base call 815 preferably use an alphabet of greater than four characters. For example, the set of final base calls may use or include any of the following characters: A, C, G, T, 5mC, 5hmC, and others, including, e.g., any of the IUPAC codes. The set of final base calls informs of several features of the invention. Those base calls represent the nitrogenous bases and modifications (such methylation or hydroxymethylation) to those nitrogenous bases. Pro-Seq allows sequencing platforms to determine the order of nitrogenous bases and to also identify modification and their positions for an input fragment of DNA. The described methods work when there is even but one single fragment, and thus are suited to cell-free DNA (cfDNA) such as cfDNA from blood or plasma, which may include circulating tumor DNA (ctDNA). The Pro-Seq process carries a built in error correction mechanism to address, and correct for, DNA lesions, polymerase error, and sequencing error. The linked strands in Pro-Seq provide for those results and all of the associated levels of information in one workflow—one single library preparation and sequencing instrument run.

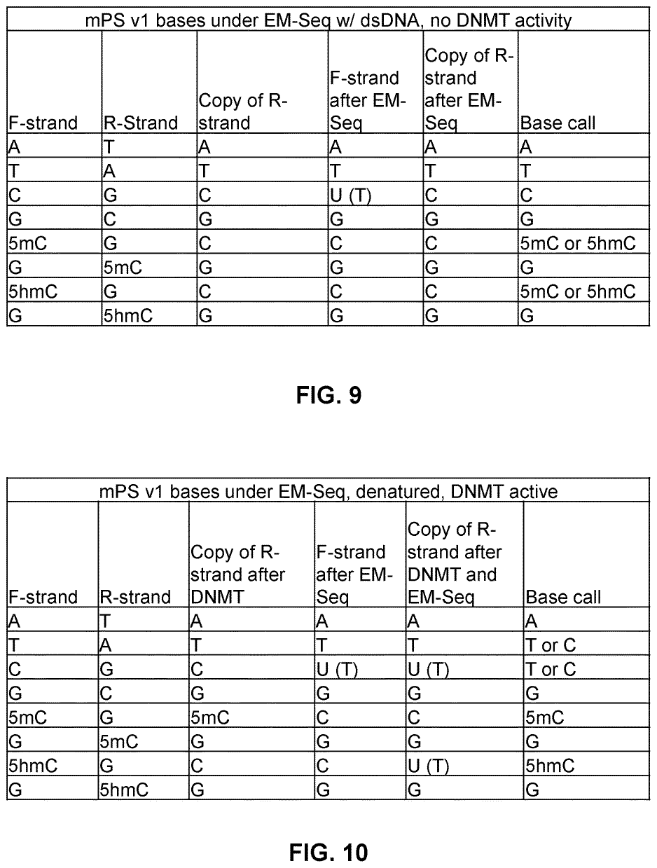

FIG. 9 is a table showing the bases, modifications, and base calls in the various strands when mPS v1 is performed on the complex 431 and without a denaturing step and no DNMT activity. In the table, the right-most column is the base-call made by the analysis unit through the analysis of the bases present after the EM-Seq reactions. As shown, after the EM-Seq reactions, in the third position, the forward-strand has a U (which is read as T) and the copy of the reverse strand has a C. By comparison, the fifth position included a 5mC in the original first strand 403 and a 5hmC at the seventh position. The TET and BGT enzymes protected those from deamination so, after EM-Seq, they were read as C in all sequence reads. The analysis unit calls each of those positions as 5mC or 5hmC due to the C base call in those positions in all sequence reads. In the table, here could be additional base combination present in the “F-strand after EM-Seq” and the “Copy of the R-strand after EM-Seq”, which base combinations are not drawn on the table. There are potential scenarios in which DNA damage, polymerase error, and/or sequencing error produce an apparent combination of bases not shown in the table. A sequencing instrument or its connected analysis unit may include the program logic to implement error correction, reflecting the duplex error correction provided by the Pro-Seq workflow, and output final base calls (including optionally flagging positions as “error” or using- or N or indicated a base as not callable) to indicate that the bases present in one position tend to indicate an error or other issue with the inputs.

FIG. 10 is a table showing the bases, modifications, and base calls when the complex is treated with DNMT. Position five of the forward strand included 5mC so the copy of the reverse strand initially included C, but DNMT methylated that C to 5mC. Position seven of the forward strand initially included 5hmC so the copy of the reverse strand included C at that position. The EM-Seq reactions protected the 5mC at position 5 in the copy of the reverse strand but converted the C at position seven to U, which is read as T. Due to the reactions, T and C in the original forward strand are both read as T, so a T among all sequence reads must be called as T or C. However, a C across all sequence reads may be called as 5mC, and a mixture of C and T among the sequence reads is called as 5hmC at that position in the original forward strand.

There are a few patterns of mixed base calls that could appear among the sequence reads and those are omitted from the Tables for clarity. In essence, if polymerase error or other unexpected enzymatic or chemical activity creates a mixture of read-specific base-calls that are not substantially represented by a set of outcomes on the Tables, the analysis unit may be programmed to report an error or to represent the base with an ambiguity code such as N.

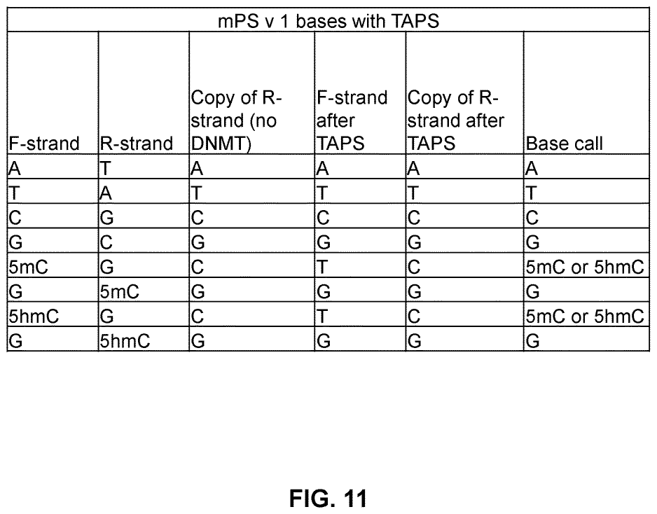

FIG. 11 shows bases and base modifications when mPS v1 is performed using TAPS. In TAPS reactions, all 5mC and 5hmC are ultimately converted to dihydrouracil (DHU). Here, 5mC and 5hmC in the reverse strand are initially copied as G, and later sequenced as G. However, 5mC and 5hmC in the forward strand become DHU, which is sequenced as T. Corresponding positions in the reverse strand are G, so the copy of the reverse strand includes C at those positions. Due to that, the sequence reads contain a mixture of T and C base calls at positions that the analysis unit will call as 5mC or 5hmC for the forward strand.

With reference to those illustrations and examples, and with reference to additional versions of methyl proximity sequencing provided herein, the disclosure provides a description of methods for methyl proximity sequencing.

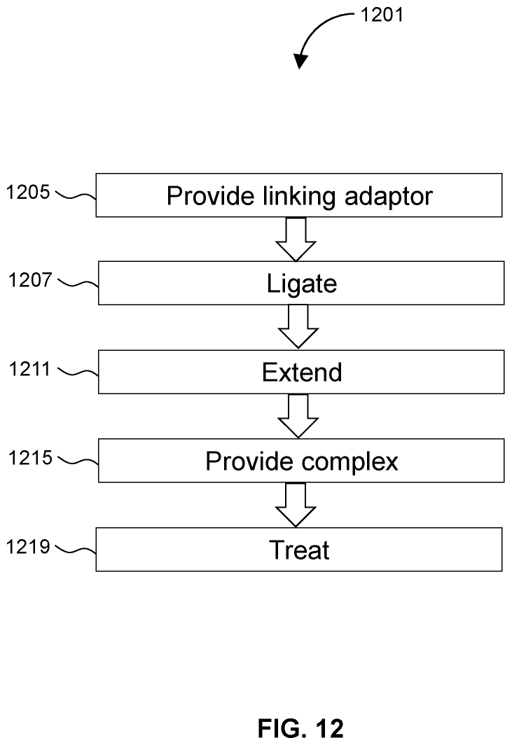

FIG. 12 shows steps of a method 1201 for identifying modified bases. The method includes providing 1205 a linking adaptor in which a first oligo is linked to a primer annealed to a second oligo. The method 1201 includes ligating 1207 the first oligo of the linking adaptor to a first strand (or forward strand) of a DNA fragment. The primer is extended 1211 to make a copy of a second strand (or reverse strand) of the fragment. This ligation and extension provides 1215 a complex that includes: the first strand ligated to the first oligo, the first oligo linked or attached to the primer wherein the primer is extended into a product that includes a copy of the second strand, and the second strand annealed to the product and ligated to the second oligo. One or more modification specific treatments are performed 1219 on the complex. For example, preferably at least a first cytosine in the first, forward strand is converted into a uracil. In the complex, the first strand and the copy of the second strand are preferably linked together at 5′ regions thereof and have the same sequence of bases in the same order except that the uracil in the first strand corresponds to a matched cytosine in copy of the second strand. The method 1201 preferably includes sequencing the first strand to yield first sequence reads and sequencing the copy of the second strand to yield second sequence reads, wherein the first sequence reads represent the first cytosine as T and the matched cytosine as C. Methods may include protecting at least one modified cytosine in the first strand from conversion to uracil. The method 1201 may include—prior to the converting step—methylating, with a methyltransferase, a second cytosine that is in the copy of the second strand, base-paired with a methylated CpG dinucleotide in the second strand, to yield a 5mC in the copy of the second strand. The method 1201 may include amplifying the first strand and the copy of the second strand to yield amplification products in which the uracil is represented as a T and the second cytosine is represented as a C. The method 1201 may include amplifying the first strand and the copy of the second strand using a plurality of primers linked to a solid support to form a cluster on the solid support. The method 1201 may include conducting a sequencing reaction on the cluster to generate sequence reads, wherein the sequence reads contain (i) a mixture of T and C base-calls at a position corresponding the first cytosine, and (ii) C base calls for the second cytosine.

In the method 1201, the fragment may be from a sample from a subject and method 1201 may include mapping the sequence reads and the positions of cytosine methylation to reference data and identifying patterns of cytosine methylation in genetic loci of the subject. The sample may be from a tumor and the fragment may include tumor DNA and the identifying step may provide tumor-specific patterns of cytosine methylation. The method 1201 may include comparing the tumor-specific patterns of cytosine methylation to a cancer database or atlas for the detection of evidence of cancer in the subject. The method 1201 may include subsequently assaying a post-treatment sample from the subject for persistence of the patterns of cytosine methylation as evidence of minimal residual disease. The sample may be a formalin-fixed, paraffin embedded (FFPE) slice of tumor tissue or a tumor biopsy and the post-treatment sample may be a liquid biopsy sample and the assaying step may include analysis of cell-free DNA in the liquid biopsy sample.

The method 1201 for identifying modified bases may proceed by various workflows or combinations of processes. For example, some embodiments make use of a linking adaptor (as shown) in combination with a bubble adaptor.

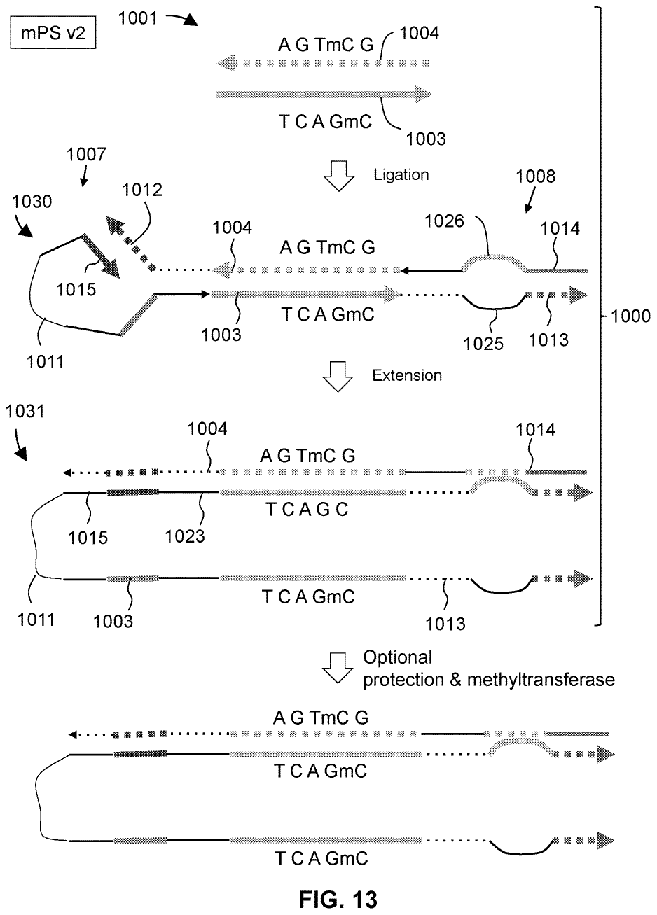

FIG. 13 shows a second version of methyl Pro-Seq, mPS v2. In an initial preparation process 1000, a fragment 1001 is exposed to a linking adaptor 1007 and a bubble adaptor 1008. The linking adaptor 1007 includes a first oligo 1011 annealed to a second oligo 1012. A primer 1015 is linked to the first oligo 1011 of the linking adaptor 1007. The bubble adaptor 1008 includes a third oligo 1013 that is partially annealed to a fourth oligo 1014. The third oligo 1013 and the fourth oligo 1014 have segments that are not reverse complementary to each other and form a bubble Within the bubble segments, the third oligo 1013 may include a universal primer binding site 1025. The fourth oligo 1014 may include its own universal primer binding site 1026.

The linking adaptor 1007 and the bubble adaptor 1008 are ligated to the fragment 1001. Specifically, the initial preparation process 1000 may include ligating the 3′ end of the first oligo 1011 to a first strand 1003 of the fragment 1001, the 5′ end of the second oligo 1012 to a second strand 1004 of the fragment 1001, a 5′ end of the third oligo 1013 to the first strand, and a 3′ end of the fourth oligo 1014 to the second strand. The primer 1015 is annealed to the second strand 1012 of the first oligo 1007 to form an intermediate complex 1030. In the preparation process 1000, the primer 1015 of the linking adaptor, which is annealed to the second oligo 1012, is extended to form a copy 1023 of the second strand and the fourth oligo, thereby forming a complex 1031.

A product of the initial preparation process 1000 is the complex 1031. The complex 1031 includes the first oligo 1011 ligated to the first strand 1003 and the third oligo 1013, with the first oligo linked to the primer 1015 and the copy 1023 of the second strand 1004 (an extension product that continues to include a new copy of the fourth oligo 1014) with the second strand 1004 annealed to the copy 1023. The complex 1031 is useful in various assays for identifying base modifications including methyl proximity sequencing such as the second version of methyl proximity sequencing (mPS v2) illustrated here.

That complex 1031 may optionally be subject to protection and methyltransferase steps. The optional protect step may use an enzyme such as BGT to glycosylate 5hmC, protecting that modification from being copied from the second strand 1004 to the copy 1023 of the second strand. An optional methyltransferase step using, e.g., DNMT1 and/or DNMT5 may be included to methylate C in hemi-methylated CpG dinucleotides present in double-stranded DNA of the complex 1031. If 5hmC are not protected (such as by glycosylation), then DNMT5 may also copy the 5hmC into the copy 1023 of the second strand.

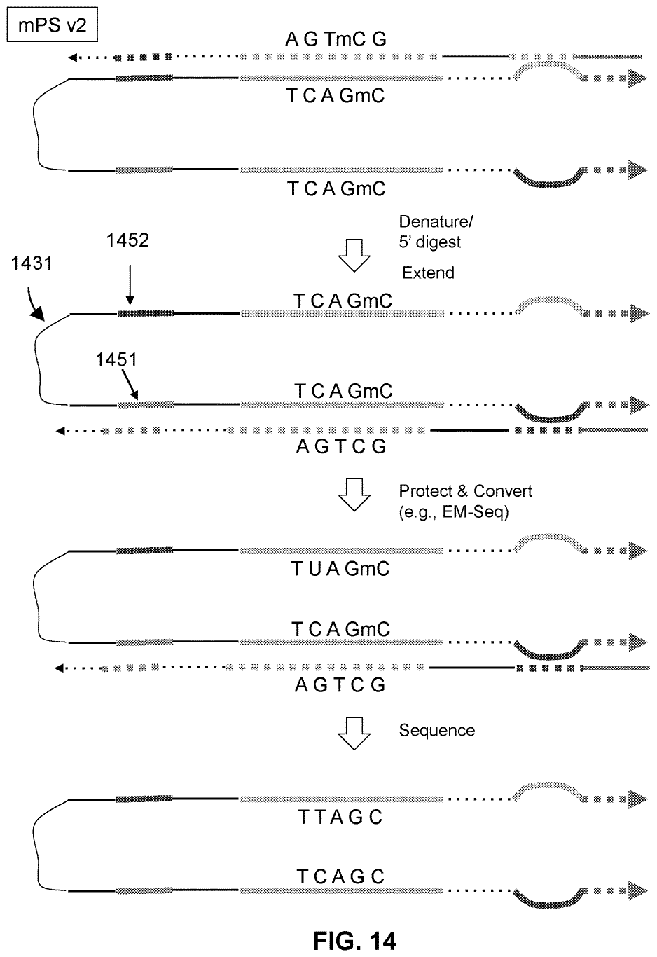

FIG. 14 shows additional steps in the second version of methyl Pro-Seq (mPS v2). After DNMT methylates in the copy of the second, reverse strand, that copy may be melted away and/or digested. Any strand digestion may be selective by which any single strand may be specifically digested off of complex 1431 using exonuclease by including modified bases, phosphorothioate linkages, or other protections within the adaptors (e.g., within the bubble adaptors as shown). Strands may optionally be protected against digestion (e.g., by protection such as modified bases or phosphorothioate linkages in the adaptors) and un-protected strands may be digested using a suitable nuclease or combinations of nucleases such as either or both of RecJf & T7 exonucleases. RecJf is a single-stranded DNA specific exonuclease that catalyzes the removal of deoxy-nucleotide monophosphates from DNA in the 5′→3′ direction. T7 exonuclease acts in the 5′ to 3′ direction, catalyzing the removal of 5′ mononucleotides from duplex DNA.

A primer may optionally be annealed to the bubble adaptor ligated to the forward strand and extended to protect the forward strand from conversion by APOBEC, which acts on single-stranded DNA. This forms an intermediate complex 1431 that is subject to the EM-Seq reactions. The intermediate complex 1431 may include a first primer binding site 1451 and a second primer binding site 1452 that are introduced by the linking adaptor and may be used downstream in an amplification or sequencing step. APOBEC converts C in the copy of the second, top strand into U.

Methods may include amplifying the first strand and the copy of the second strand with a plurality of copies of a primer, e.g., linked to a solid support such as a bead or flow cell to form a cluster. Methods may include conducting a sequencing reaction with the cluster.

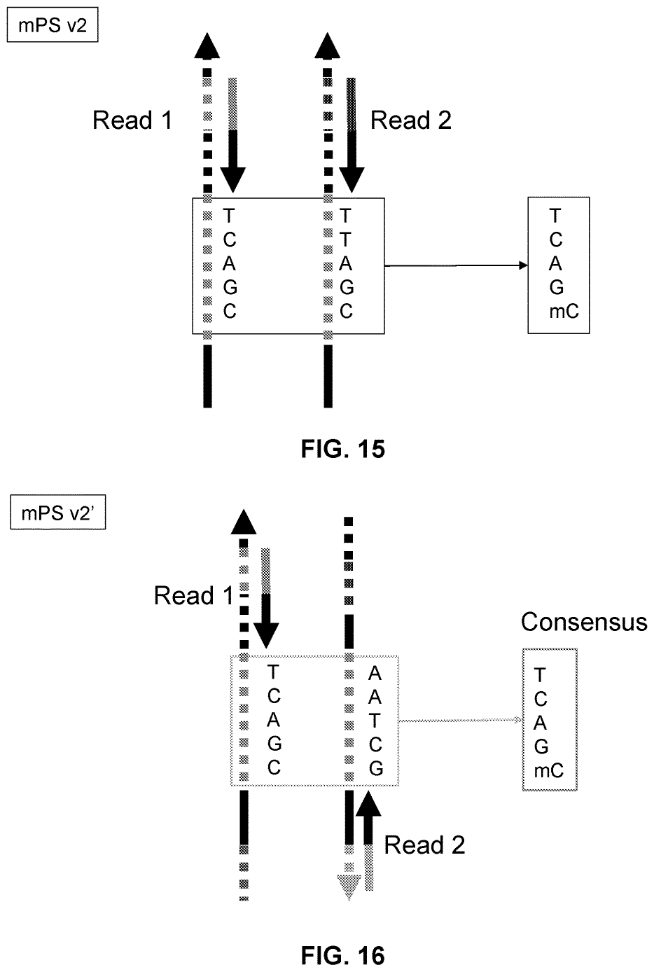

FIG. 15 illustrates a first strategy for a sequencing reaction with the cluster. Members of the cluster are sequenced using a plurality of primers that sequence in the same direction (relative to the linkage to the solid support).

FIG. 16 shows a second strategy in which the cluster is sequenced from both ends using two different primers. In numerous of the examples shown in disclosure, certain figures show a Read 1 and a Read 2 in a “same direction” on the figure, as if those reads were made conducting a sequencing-by-synthesis reaction on strands in a cluster in a same direction from the same homologous ends of strands. In FIG. 15, a Read 1 and Read 2 are shown using two primers that sequence, from the same ends and in the same direction (with respect to strands in a cluster). In FIG. 16, a Read 1 and Read 2 are shown using two primers to generate sequence reads from opposed ends of strands in a cluster. The relative position and orientation of the sequence read primer binding and direction of sequencing reaction is flexible and those may be conducted on a cluster in any direction suitable for the workflow of the particular embodiment. While the cluster sequencing strategies are shown here in FIG. 16 and FIG. 15 in connection with mPS v2, those read orientations and sequencing strategies are applicable to any suitable embodiment herein. Under either strategy, sequencing produces a plurality of sequence reads. Methods may include analyzing the sequence reads to independently identify patterns of 5hmC and 5mC in the fragment.

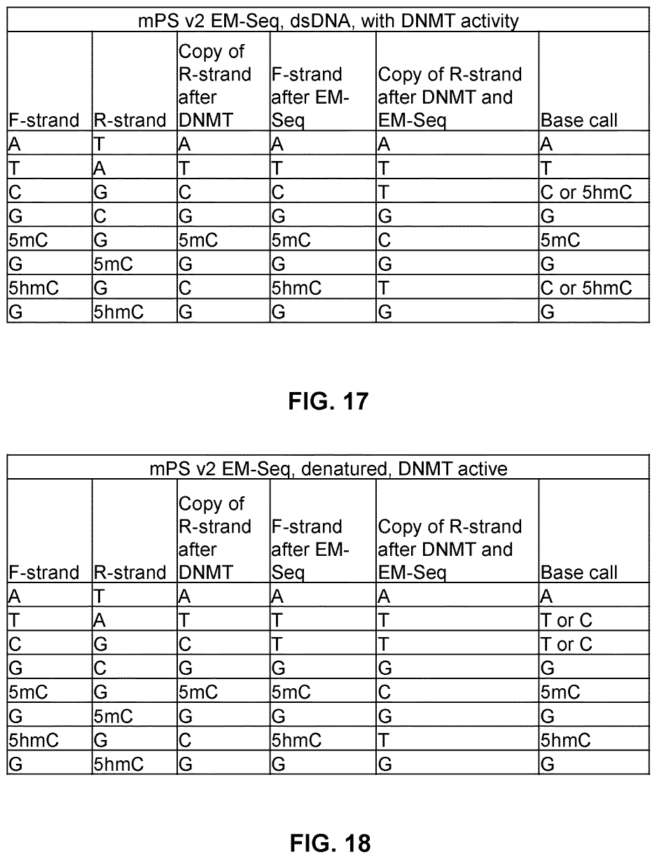

FIG. 17 is a table showing the bases, modifications, and base calls made when mPS v2 is used with DNMT and the EM-Seq reactions.

FIG. 18 is a table showing the bases and base calls made when mPS v2 is used with DNMT and the EM-Seq reactions.

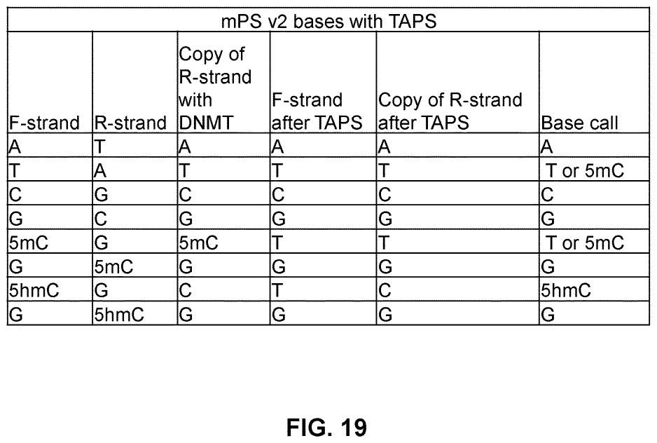

FIG. 19 is a table showing the bases and base calls made with mPS v2 and TAPS. The logic is the same for mPS v1 and the columns labeled “F-strand after (reactions)” and “R-strand after (reactions)”, as well as the columns labeled “Base call” are programed into the analysis unit. When the assay is performed, the set of reactions being performed is entered into the analysis unit, which then refers to the appropriate corresponding Table. The analysis unit compared the base calls among the input sequence reads to the “F-strand after (reactions)” and “R-strand after (reactions)” entries and outputs, to the user, the corresponding base call for the fragment.

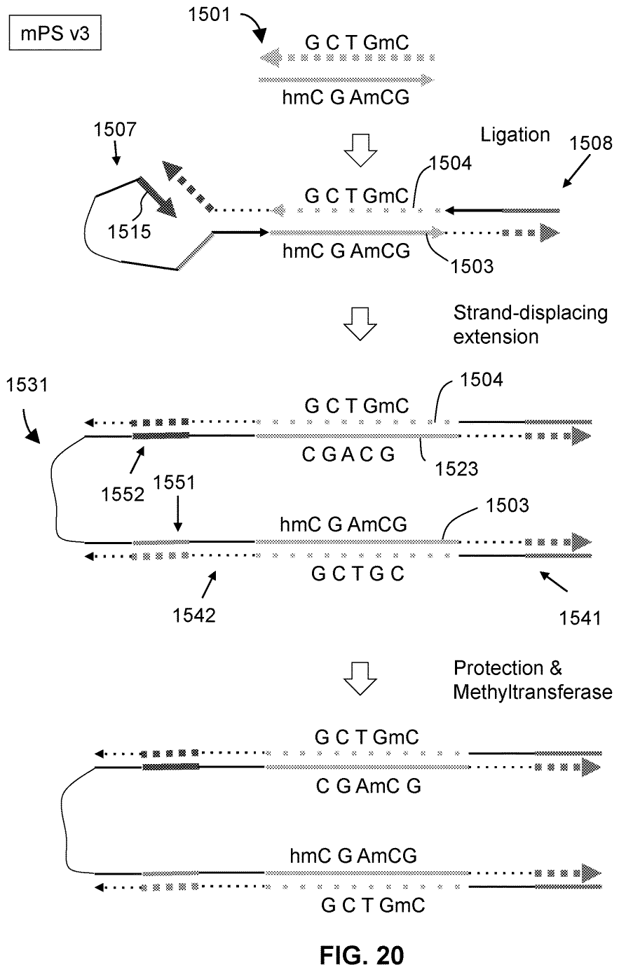

FIG. 20 shows steps of a third version of methyl Pro-Seq, mPS v3. As shown, mPS v3 includes exposing a fragment 1501 to adaptors that include a linking adaptor 1507 and a second adaptor 1508. The linking adaptor 1507 includes a double-stranded portion in which a 3′ end of a first oligo is annealed to a 5′ end of a second oligo, wherein the first oligo is linked to a primer 1515 complementary to the second oligo. The second adaptor 1508 includes a third oligo annealed to a fourth oligo. The depicted workflow proceeds by ligating the 3′ end of the first oligo to a first strand of the fragment, the 5′ end of the second oligo to a second strand of the fragment, a 5′ end of the third oligo to the first strand, and a 3′ end of the fourth oligo to the second strand.

The primer 1515, annealed to the second oligo of the linking adaptor 1507, is extended to form an extension product that includes a copy 1523 of the second strand 1504 and the fourth oligo, thereby forming a complex 1531.

The complex 1531 includes the first oligo ligated to the first strand and the third oligo, with first oligo linked to the primer and the extension product. Methods may include annealing a universal primer 1541 to the third oligo and extending the universal primer 1541 to form a copy 1542 of the first strand annealed to the first strand 1503, whereby the complex 1531 includes the first strand 1503 annealed to the copy 1542 of the first strand and the copy 1523 of the second strand annealed to the second strand 1504. The complex 1531 may include a first primer binding site 1551 and a second primer binding site 1552 from the linking adaptor 1507 (e.g., each different or the same and suitable for being annealed to by universal primers or sequencing primers for downstream amplification and/or sequence steps, preferably the two are different and are used downstream with two different sequencing primers).

The mPS v3 workflow may include a step of protecting 5hmC. The workflow preferably includes treating the complex with a methyltransferase to methylate a cytosine in the copy of the second strand paired with a methylated CpG dinucleotide in the second strand.

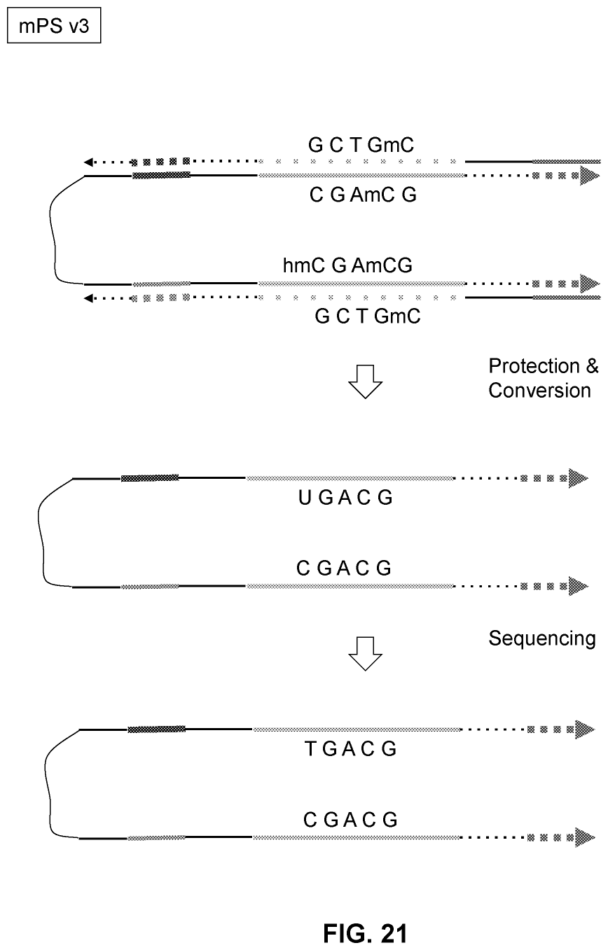

FIG. 21 shows subsequent steps of the third version of methyl Pro-Seq, mPS v3. After the methylation step transferred methylation at hemimethylated CpG sites to methylate at least one C to 5mC, those are protected, and APOBEC or similar is used to convert C to U (after denaturing). The third version of methyl Pro-Seq, mPS v3, may include conducting a sequencing reaction with the first strand and the copy of the second strand to generate sequence reads.

FIG. 22 shows the individual sequence reads (each drawn against the strand being sequenced) and the base call that would be made for each position under this mPS v3 workflow. As shown, a 5hmC in the first strand yields a mixture of T and C base calls and a 5mC in the first strand, in a position corresponding to the methylated CpG dinucleotide in the second strand, generates C base calls across the sequence reads.

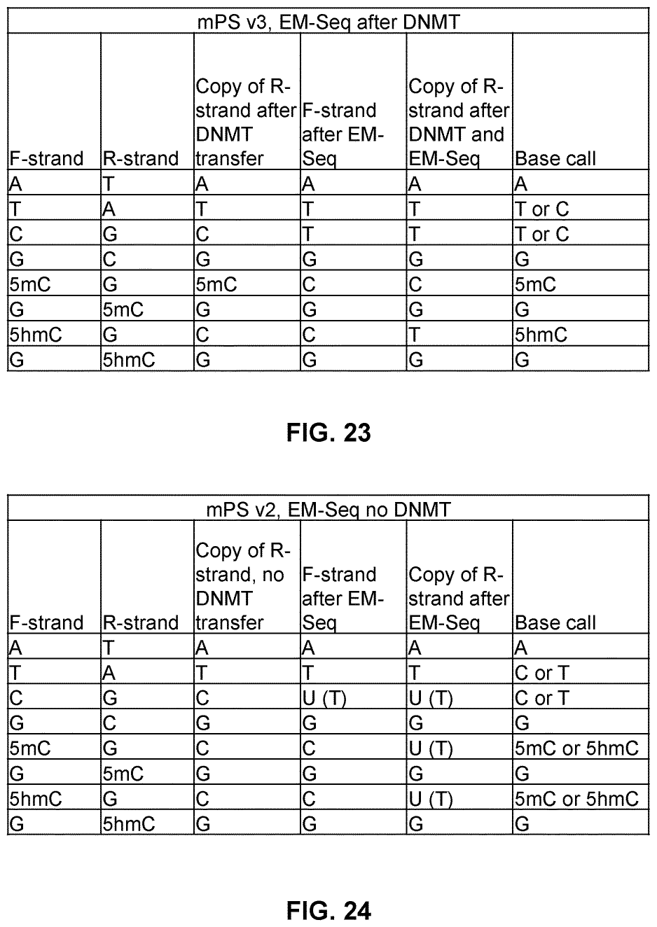

FIG. 23 gives more comprehensive results of the bases and base call when mPS v3 is used with EM-Seq reactions after DNMT.

FIG. 24 shows bases and base call when mPS v3 is used with EM-Seq reactions without a DNMT step.

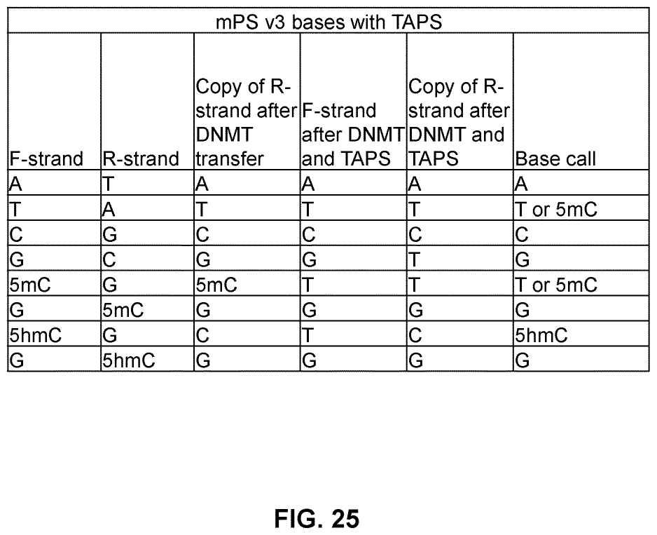

FIG. 25 shows bases and base call when mPS v3 is used with TAPS.

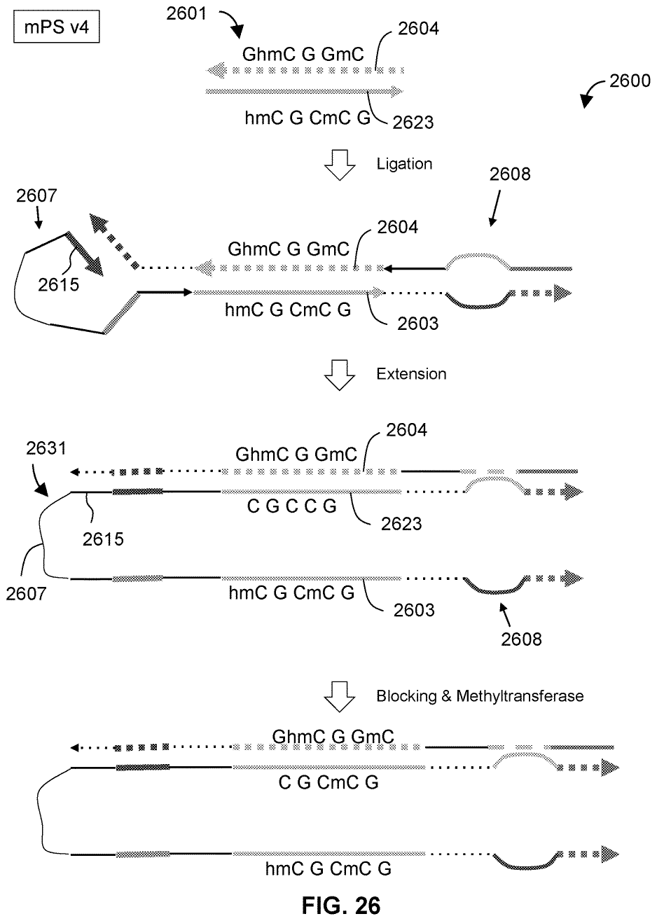

FIG. 26 shows a fourth version of methylation proximity sequencing, mPS v4. The fourth version (mPS v4) may include initial preparation process 2600. The process 2600 includes ligating adaptors to a fragment 2601. The adaptors include a linking adaptor 2607 and a bubble adaptor 2608. As with linking adaptors discussed above, the linking adaptor 2607 includes a primer 2615 connected to a first oligo and the primer is annealed to, or will anneal to, a second oligo of the linking adaptor 2607. The bubble adaptor 2608 includes a double-stranded ligation end, a portion where its third oligo and fourth oligo are not complementary to each other, and a double-stranded distal segment (which may further optionally include a Y-end or a sticky end, not shown). The linking adaptor 2607 is ligated to the fragment 2601 so that the first oligo of the linking adaptor is covalently attached to a first, or forward, strand 2603 of the fragment 2601 and the second oligo of the linking adaptor 2607 is covalently attached to a second, or reverse, strand 2604 of the fragment 2601. After ligation of the linking adaptor 2607 and the bubble adaptor 2608 to the fragment 2601, the primer 2615 is extended to make a copy of the second strand 2604 (or reverse strand) of the fragment 2601. Extension of a primer 2615 in the linking adaptor 2607 forms a complex 2631.

The complex 2631 includes the first strand 2603 of the fragment 2601 linked to a copy 2623 of a second strand 2604 of the fragment in a duplex with the second strand 2604. The first strand 2603 is linked to the copy 2623 of the second strand via the first oligo of the linking adaptor 2607 and the now extended primer 2615. The second oligo of the linking primer 2607 is now a covalent portion of the copy 2623. The third oligo and the fourth oligo of the bubble primer 2608 are attached at distal ends of the first strand 2603 and the copy 2623. Due to the segments within the third oligo and the fourth oligo that are not complementary to each other, each of those segments may include dissimilar universal primer binding sites, allowing each linked strand of the complex 2631 to be copied, independently of the other, with a universal primer uniquely cognate to the strand of the bubble primer 2608.

As shown within the complex 2631, a first or forward strand 2603 of the fragment 2603 is present as single-stranded DNA (ssDNA). The copy 2623 of the second or reverse strand is annealed to the second strand 2604 of the fragment 2601. The third and fourth oligos of the bubble adaptor 1608 are attached, providing unique universal primer binding sites to the first stand 2603 and the copy 2623. The complex 2631 is the product of the process 2600, and is subsequently used in the subsequent analytical steps of the fourth version of methyl proximity sequencing (mPS v4) and optional a fifth version (mPS v5) or others. Optionally 5hmC may be protected.

In the fourth version (mPS v4), as shown, the complex 2631 may be optionally exposed to methyltransferase such as DNMT1 or DNMT5. The methyltransferase methylates Cs present in hemi-methylated CpG dinucleotides in the dsDNA that includes the copy 2623 of the second strand.

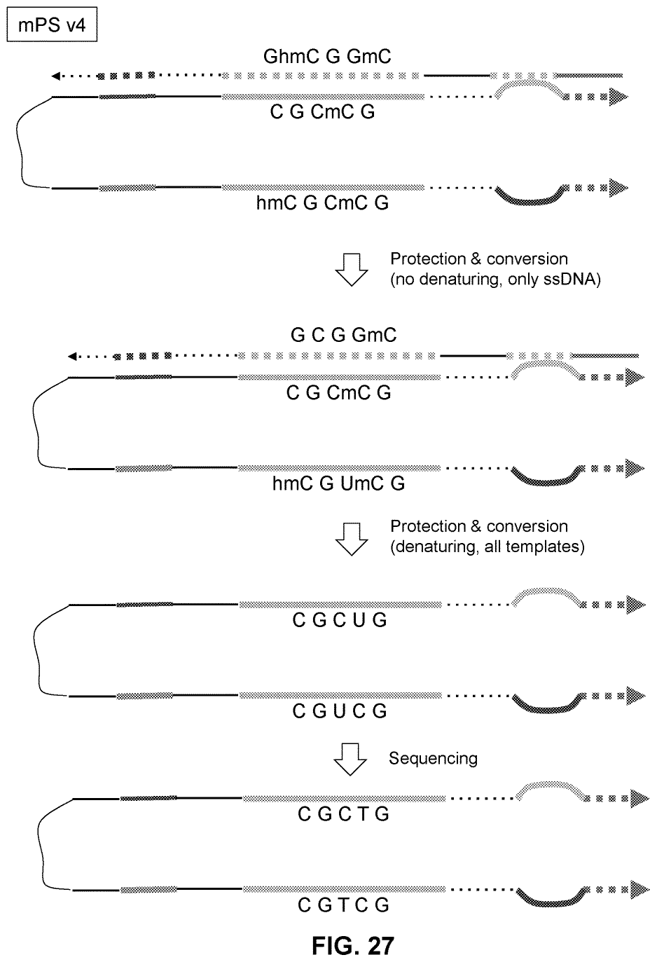

FIG. 27 shows the continuation of the fourth version of methylation proximity sequencing, mPS v4. The EM-Seq reaction converts a non-dinucleotide C in the first strand to U. The complex is denatured, and protection and subsequent conversion reactions convert C in the copy of the second strand. The products are subject to sequencing reactions to generate sequence reads.

FIG. 28 shows the base calls from individual sequence reads in mPS v4 as well as base calls made for the fragment by the analysis unit. As shown, the analysis unit calls a 5hmC in the fragment when the individual reads all have C base calls and the analysis unit calls a 5mC or a C in the fragment where reads include a mixture of C and T.

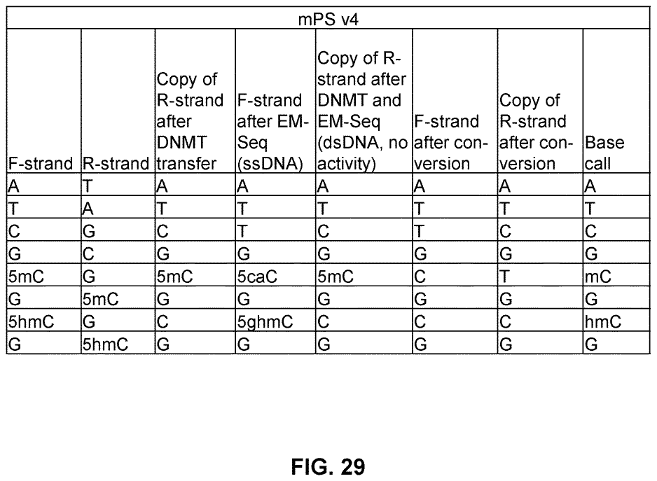

FIG. 29 is a table showing the bases and base calls involved in mPS v4.

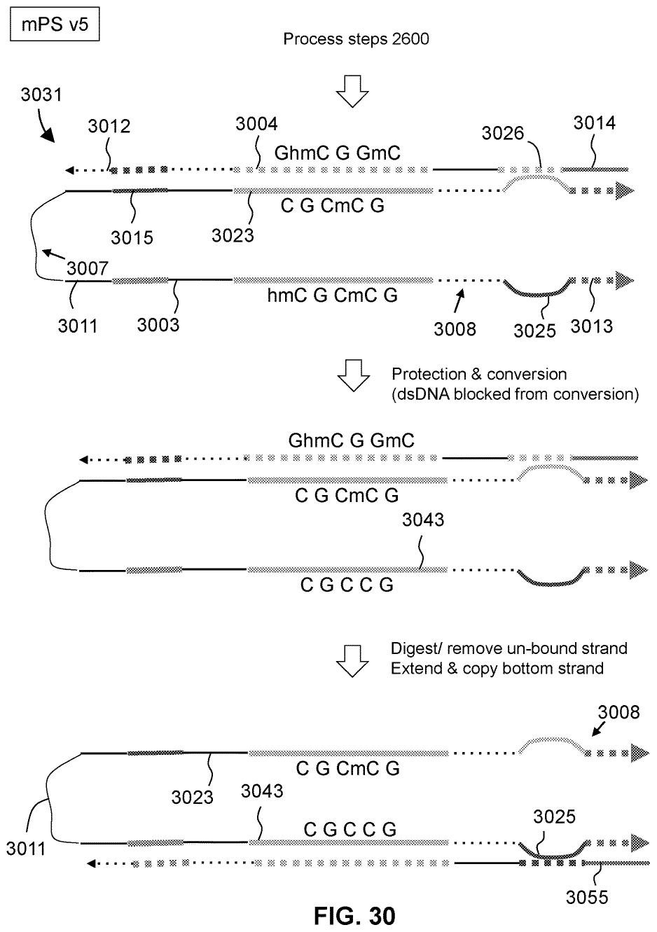

FIG. 30 illustrates steps of a fifth version of methyl proximity sequencing, mPS v5. The fifth version (mPS v5) involves creating a complex 3031. This is created in a manner similar to the creation of complex 2621 in mPS v4. That is, the process 2600 may be used to create the complex 3031.

The complex 3031 includes a linking adaptor 3007 ligated to a first strand 3003 and a copy 3023 of a second strand 3004. A first oligo 3011 of the linking oligo 3007 is ligated to the first strand and linked to a primer 3015, which is annealed to a second oligo 3012 of the linking adaptor 3007. The first strand 3003 is linked to a third oligo 3013 of a bubble adaptor 3008. the copy 3023 is annealed to a second strand 3004, which is ligated to a second oligo 3012 of the linking adaptor 3007 and a fourth oligo 3014 of the bubble adaptor 3008. The third oligo 3013 of the bubble adaptor 3008 includes one universal primer binding site 3025, and the fourth oligo 3014 includes a second universal primer binding site 3016 (now copied into the copy 3023).

In the fifth version (mPS v5), the complex 3031 is subject to the protection and conversion reactions of a first EM-Seq step, which leaves a converted strand 3043.

The fifth version (mPS v5) proceeds by digesting or removing the second strand 3004 of the fragment that was annealed to the copy 3023 of the second strand.

This digestion or removal step leaves two single stranded DNA molecules linked at their 5′ ends by the first oligo 3011 and having the same sequence of bases but for certain cytosine modifications. A universal primer 3055 is annealed to the universal primer binding site 3025 in the third oligo 3013 of the bubble adaptor 3008. The universal primer 3055 is extended to copy the converted strand 3043.

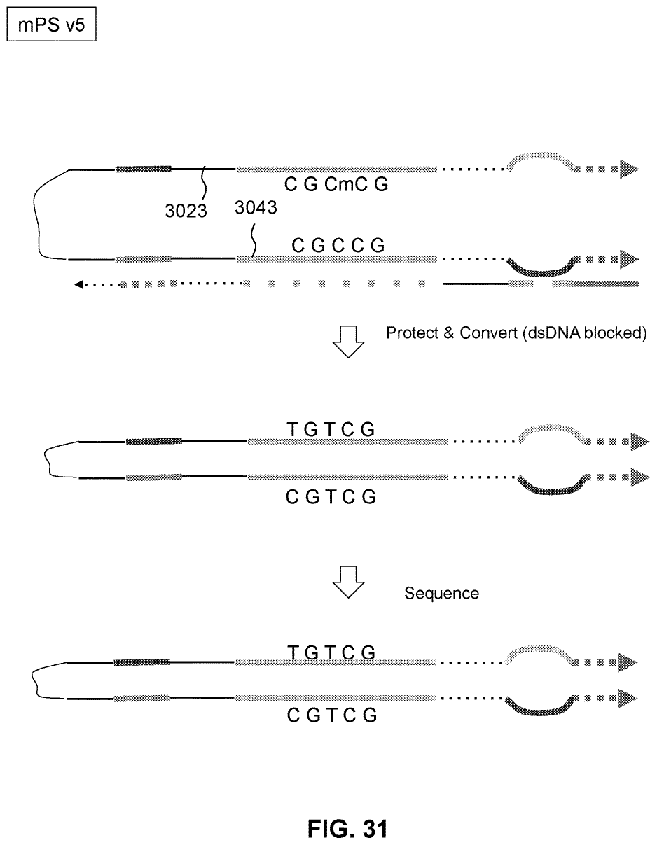

FIG. 31 shows further steps of the fifth version of methyl proximity sequencing (mPS v5) in which the complex is subject to protection and conversion (e.g., TET1 and Beta-GT) followed by APOBEC, in which non methylated C is converted to U (which read as T). The resulting molecular complex is subject to amplification and sequencing as described elsewhere herein.

Amplification may proceed by copying the linked strands of a complex using primers linked to a solid support. As shown in the method 600, primers 603 may be linked together by a linker 609 such as the surface of a flow cell. Embodiments of the invention include other suitable approaches to amplification including, for example, amplification onto beads optionally within an aqueous compartment such as a droplet.

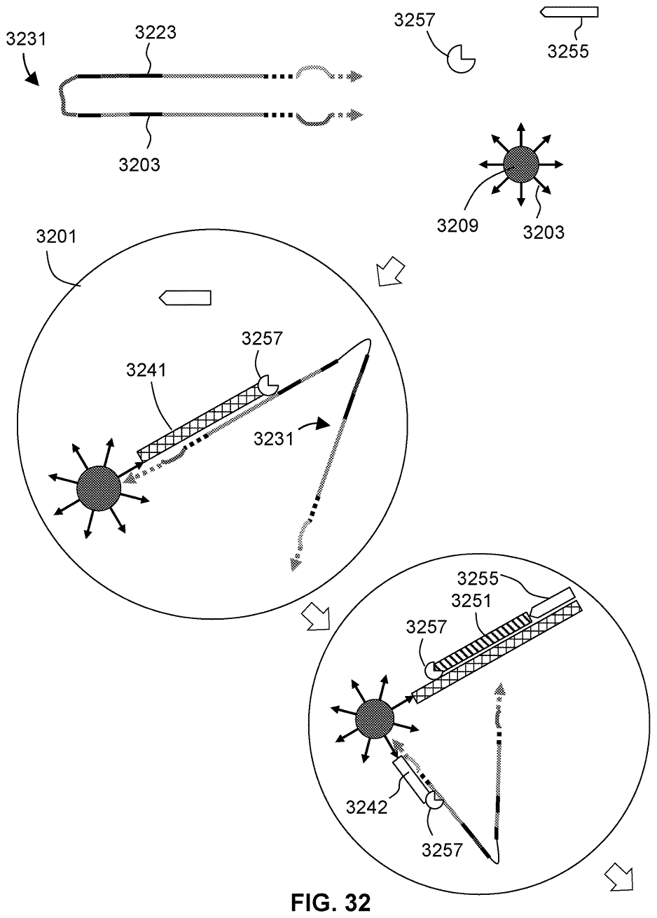

FIG. 32 shows first steps of a compartment workflow. Some embodiments use an aqueous compartment 3201 such as a droplet or well to encapsulate a post-conversion complex 3231 with a primer-linked bead 3209, second primer 3255, polymerase 3257, and other reagents (e.g., dNTPs, co-factors, etc.). The compartment workflow may include amplification within the compartment 3201, e.g., droplets such as microfluidic droplets or the droplets of an emulsion. A post conversion complex 3231 typically has an optionally modified first, forward strand 3203 linked to an optionally modified copy 3223 of a second, reverse strand. The post-conversion complex 3231, a bead 3209 decorated with primers 3203 and second primer 3255 are introduced.

The primer-linked bead 3209, the post conversion complex 3231, and the second primers 3255 are encapsulated in an aqueous compartment 3201. Any suitable aqueous compartment 3201 may be used. Suitable aqueous compartments 3261 include droplets (e.g., of an emulsion or formed on a microfluidic platform), wells in a plate (such as a multi-well plate, a microtiter plate or a picotiter plate), fluidic harbors, electrostatically controlled droplets on a surface, individual tubes (e.g., microcentrifuge tubes), or any other suitable compartment. Preferred embodiments use aqueous droplets in an immiscible carrier fluid such as droplet of a water-in-oil emulsion. Such compartments may be thermocycled, e.g., by loading a plurality of emulsion droplets within a tube into a thermocycler or by flowing droplets in a channel of a microfluidic platform through a sequence of temperature controlled regions. The compartment is preferably provided with a PCR master mix, as is known in the art (e.g., dNTPs, co-factors, polymerase, etc.).

In the first steps of the compartment workflow, a primer 3203 on the bead 3209 anneals to a strand of the complex 3231 and is extended by polymerase 3257 to form a first bead-linked extension product 3241. At a next step, the second primer 3255 anneals to the first bead-linked extension product 3241 and is extended by polymerase 3257 to make a single-strand intermediate 3251. Additionally and/or separately, the complex 3231 is caught by a primer 3203 that is extended to make a second bead-linked extension product 3242.

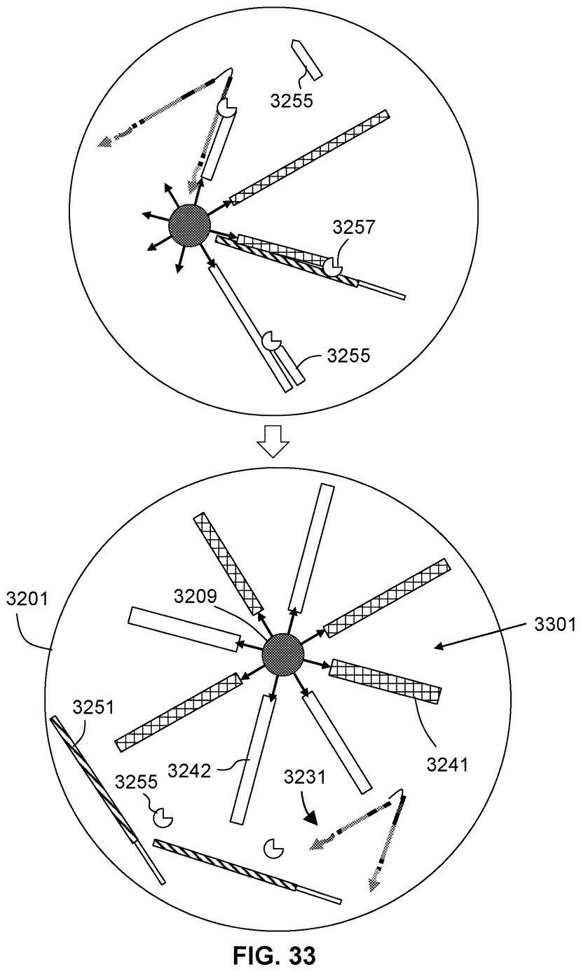

FIG. 33 shows additional steps of the compartment workflow. The second primer 3255 anneals to the second bead-linked extension product 3242 and is extended to make another single-strand intermediate 3251. Another primer 3203 on the bead 3209 captures the single-strand intermediate 3251 that was first made, and is extended to make another first bead-linked extension product 3241. As drawn, the complex 3231 is also being caught by another primer 3203 that is being extended to make another second bead-linked extension product 3242.

The product of these reactions of the compartment workflow is the bead 3209 that is linked to a plurality of the first bead-linked extension products 3241 and the second bead-linked extension products 3242 in the compartment 3201. The compartment 3201 may also contain the complex 3231, polymerase 3257, and single-strand intermediates 3251.

One product of these reactions of the compartment workflow is a cluster 3301 comprising a plurality of the first bead-linked extension products 3241 and the second bead-linked extension products 3242 all linked to a bead 3209. The cluster 3301 may be removed from the compartment 3201 (e.g., by breaking the droplet, which may be done chemically (e.g., detergent), mechanically (e.g., sonication or centrifugation), other techniques or a combination thereof. The other materials may be washed away. E.g., the bead 3209 may be magnetic and may be held on a magnet while the cluster 3231, polymerase 3257, and single-strand intermediates 3251 are washed away. The cluster 3301 may be isolated by washing, by microfluidic handling, by filtration, or by other means known in the art.

Such methods may be used to amplify the linked templates on a bead 3209, in a compartment 3201 such as the droplet of an emulsion. As shown, both linked templates are represented on the bead. After amplification, the bead carries a plurality of bead-linked oligonucleotides in the form of the first bead-linked extension products 3241 and the second bead-linked extension products 3242 that all have substantially the same 5′ to 3′ sequence in the same orientation. The sequence would be the same but for any base modifications that were treated differently by the mPS methods of the disclosure and but for any polymerase error that occurs during the amplification. The cluster 3301 may be released from the aqueous compartment (e.g., by breaking an emulsion using a recovery agent (e.g., a surfactant) and/or centrifugation).

The cluster 3301 may be exposed to sequencing primers e.g., in a sequencing instrument. The cluster 3301 may be subject to a sequencing reaction, such as a sequencing reaction that is conducted in a well or at a location over a flow cell. Semiconductor sequencing is conducted with bead-linked oligonucleotides in instrument sold under the trademark ION TORRENT by Thermo Fisher Scientific (Waltham, MA). Pyrophosphate sequencing is conducted with bead-linked oligonucleotides in instruments sold under the trademark ROCHE/454 by Roche Sequencing Solutions, Inc. (Indianapolis, IN). Bead-linked oligonucleotides may be subject to a sequence reaction at a location over a flow cell in instruments sold under the trademark UG 100 by Ultima Genomics, Inc. (Fremont, CA). A cluster may be sequenced on a flow cell in instruments sold under the trademark ILLUMINA by Illumina, Inc. (San Diego, CA).

Methods and processes of the disclosure may be used for profiling epigenetic modifications within genetic material while also sequencing the genetic material. The methods may be used with any suitable research or clinical samples. For example, a fragment may be from a clinical sample from a subject and methods herein may include methyl proximity sequencing to generate sequence reads mapping the sequence reads to reference data and independently identifying loci of 5hmC and 5mC in the subject (wherein the mapping is easier to perform than in standard e.g. EM-seq workflows, where the libraries are effectively converted to a 3 base genome, as C's are much less frequently present in the sample). In certain embodiments, the sample is from a tumor (e.g., an FFPE tumor slice) and the fragment may be tumor DNA. The identifying step may provide tumor-specific patterns of 5hmC and 5mC. Methods may include comparing the tumor-specific patterns of 5hmC and 5mC, and genomic alterations, to a cancer database or atlas for the detection of evidence of cancer in the subject. Methods may include subsequently assaying a post-treatment sample from the subject for persistence of the patterns of 5hmC and 5mC, and genomics alterations, as evidence of minimal residual disease (MRD). The post-treatment sample may be a liquid biopsy sample and the assaying step may involve analysis of cell-free DNA in the liquid biopsy sample.

Certain other features and workflows may be used within methods of the invention.

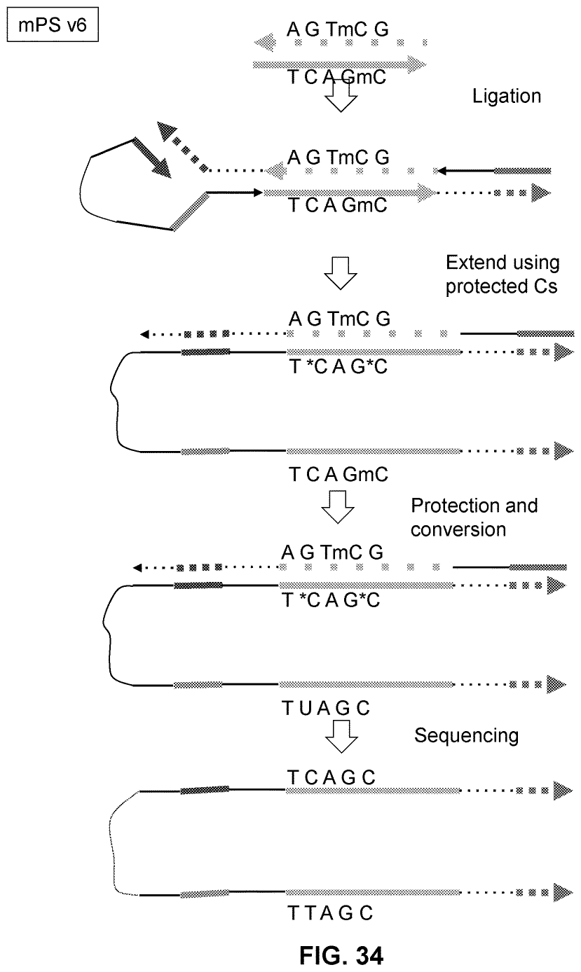

FIG. 34 illustrates a sixth version of methyl proximity sequencing (mPS v6). In the sixth version, a fragment is ligated to a linking adaptor and a second adaptor. The primer of the linking adaptor is extended using protected nucleotides (e.g. 5-methyl-dCTP, carboxy-dCTP, hydroxy-dCTP and propargylamino-dCTP), during strand displacing extension (e.g. first extension). This prevents conversion of the copied strands during BS/EM-Seq, or forces C→T conversion for TAPS. Extension with protected bases could be used in place of ‘no denaturing’ (i.e. retaining dsDNA) to inhibit deamination activity on a particular strand. As with other versions, the initial complex may be subject to optional protection & methyltransferase (e.g. DNMT1, DNMT5). The complex is subject to protection & conversion (e.g. the EM-Seq reactions).

The second adaptor could alternatively be a bubble adaptor. The method may include denaturing or digest (from 5′) one strand. The linked fragments are protected from, e.g., RecJf & T7 exonucleases, which may be used. Using a primer specific for a bubble adaptor, methods may include extension to copy a second stand, after which the methods may include protection and conversion (e.g. the EM-Seq reactions).

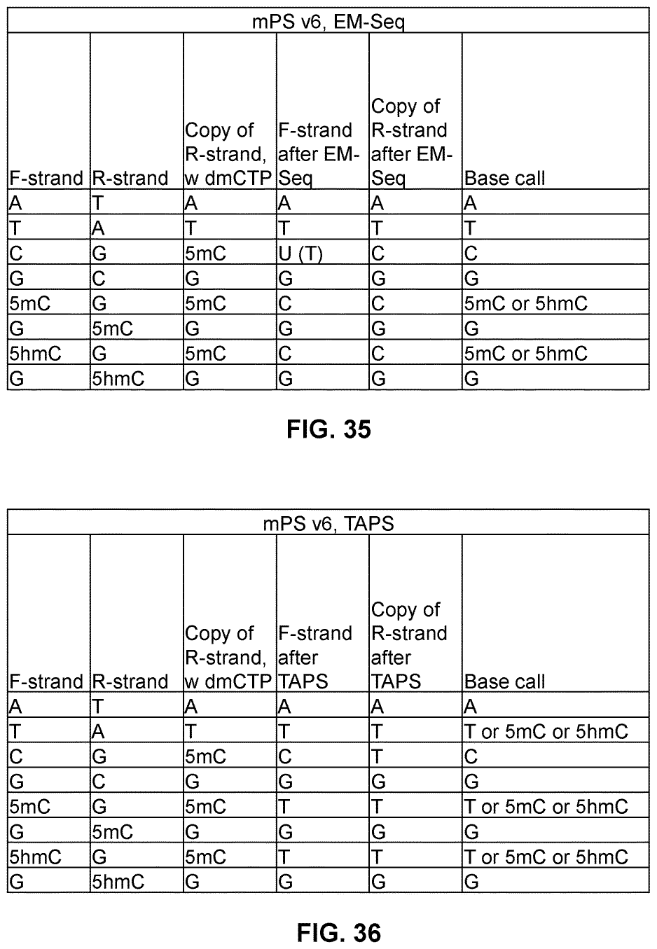

FIG. 35 is a table of bases and the base calls for version six of methyl proximity sequencing (mPS v6) when the EM-Seq reactions are used.

FIG. 36 is a table of bases and the base calls for version six of methyl proximity sequencing (mPS v6) when the TAPS reactions are used.

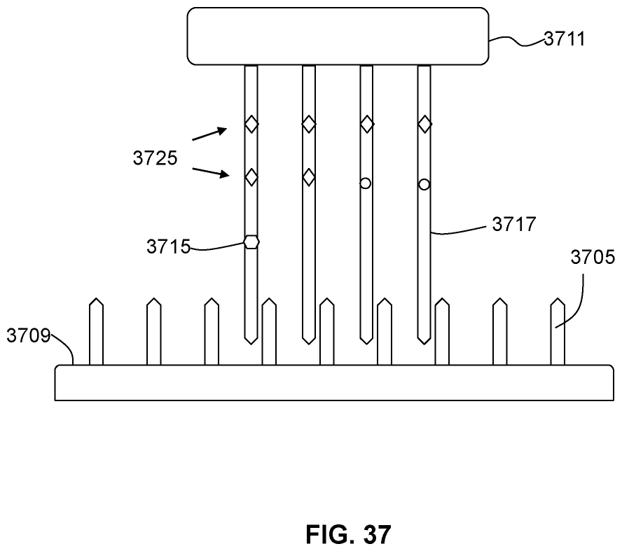

FIG. 37 illustrates how linking primers used in proximity sequencing allow one to not only specifically profile modified bases with single-base resolution but also provide a built-in mechanism for polymerase error correction. As shown, methods of the invention provide polynucleotides 3717 that have substantially the same base order (but for modified bases 3725) in the same 5′ to 3′ orientation linked together by a linker 3711, where the polynucleotides 3717 may already be a cluster for a sequencing reaction or may be annealed to primers 3705 on a solid substrate 3709 for amplification into a sequence reaction An early polymerase error 3715 will not appear among sequencing reads in the same distribution as base calls for the modified bases 3725. The ratio of sequence reads that include base call for a C versus a modified C and/or the combinations of base calls may be different from a ratio of sequence reads that includes a base call or the combinations of bases that are called in result of polymerase error. Additionally, looking back at the Tables of base calls, combinations of base calls that are not specifically listed in the corresponding table indicate an error. Errors such as DNA lesions, polymerase errors during library preparation, and sequencing errors may be evident as different combinations of bases among the sequence reads or different ratios of base calls among those reads. In one example, a modified C will create a base call among about 50% of the sequence reads. A connected analysis unit may include program logic in the computer system that uses stored information about expected ratios of base calls for modified bases to identify positions that reflect modified bases in the original template and positions that were subject to sequencing error during amplification.

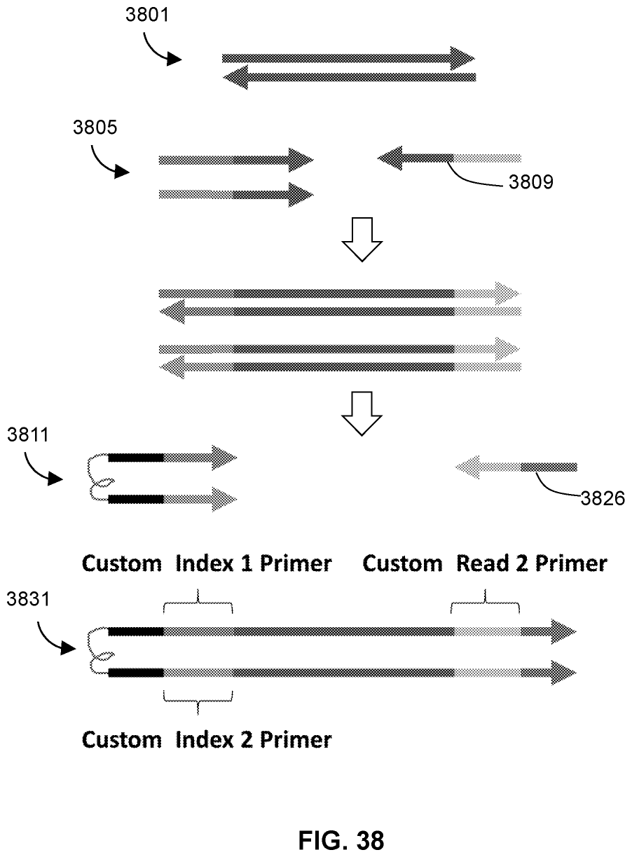

FIG. 38 illustrates an approach to create a complex that will include two independently made copies of a template. A fragment 3801 is amplified with reverse primer 3809 and a mixture of forward primers 3805 with two different universal tails. The reverse primer 3809 includes a unique universal tail. A pair of linked universal primers 3811 are introduced that do not match each other but in which each matches one of the two different universal tails. A suitable reverse universal primer 3826 is introduced. Primer extension here with a PCR mix produces a complex 3831. The complex 3831 includes two strands with the same base sequence in the same 5′ to 3′ order (“in phase”) and linked at their 5′ ends. Each strand is an independent copy of the fragment 3801. Assuming equal and unbiased primer use, this workflow as shown will produce linked copies originating from the sense and the antisense strand about 50% of the time. Independent means that any polymerase reaction that contributed the synthesis of one strand did not contribute to the other strand. Because the copies of the complex 3831 are independent, a polymerase error in the creation of one cannot appear in the other.

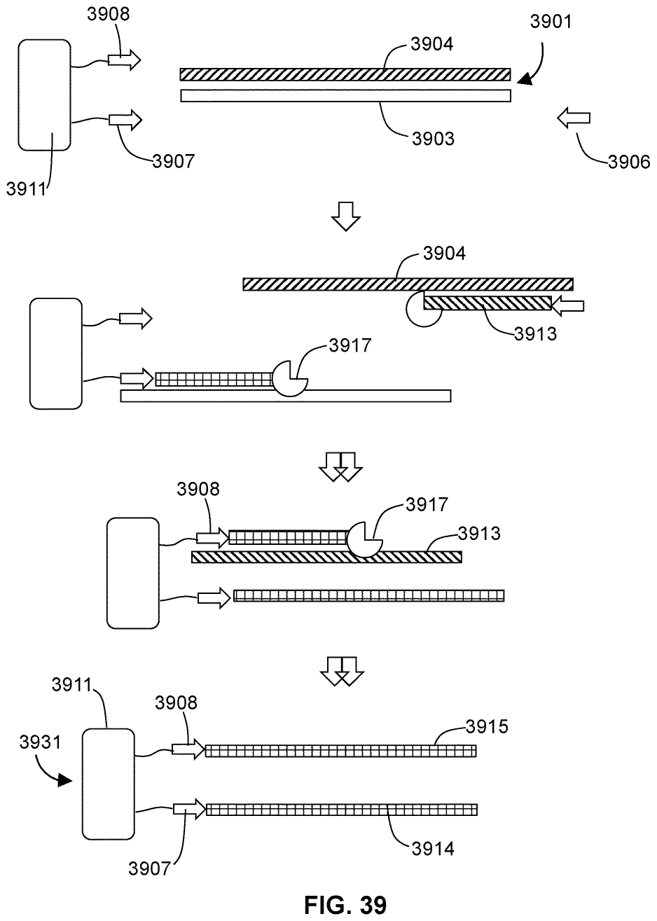

FIG. 39 shows a process for linking independent copies a forward strand and a reverse strand of the fragment 3901 in the same 5′ to 3′ orientation (“in phase”). A primer 3907 linked to a second primer 3908 via an optional linker 3911 are introduced to the fragment 3901 in which the forward strand 3903 is annealed to the reverse strand 3904. A back primer 3906 is also introduced. The primers 3907, 3908 and the back primer 3906 may be locus-specific or universal primers designed to anneal to universal sites in adaptors attached to the fragment 3901. The fragment 3901 is denatured and the primer 3907 is annealed to the forward strand 3903 while the back primer anneals to the reverse strand 3904. Polymerase 3917 copies the first strand and makes a copy 3913 of the reverse strand 3904. The copy 3913 of the reverse strand 3904 is annealed to the primer 3908, which is extended by polymerase 3917 to make a copy of a copy 3915 of the reverse strand, which is linked via the linker 3911 to the copy 3914 of the forward strand.

The depicted steps produce a complex 3931 that includes a copy 3914 of the forward strand 3903 and a copy of a copy 3915 of the reverse strand 3904. Those two strands are bound at their 5′ ends by the linker 3911. Those two strands have the same base order in the same 5′ to 3′ orientation when considered with respect to the linker 3911. The steps may be performed with a single fragment 3901, optionally in an aqueous compartment. The fragment 3901 may be ligated with one or more adaptors comprising sense-specific barcodes to confirm that both strands are represented in the complex 3931. Additionally, back primer 3906 may have a low Tm or include locked bases to promote its extension to form copy 3913. The depicted steps may be performed in a droplet and the linker may be a bead.

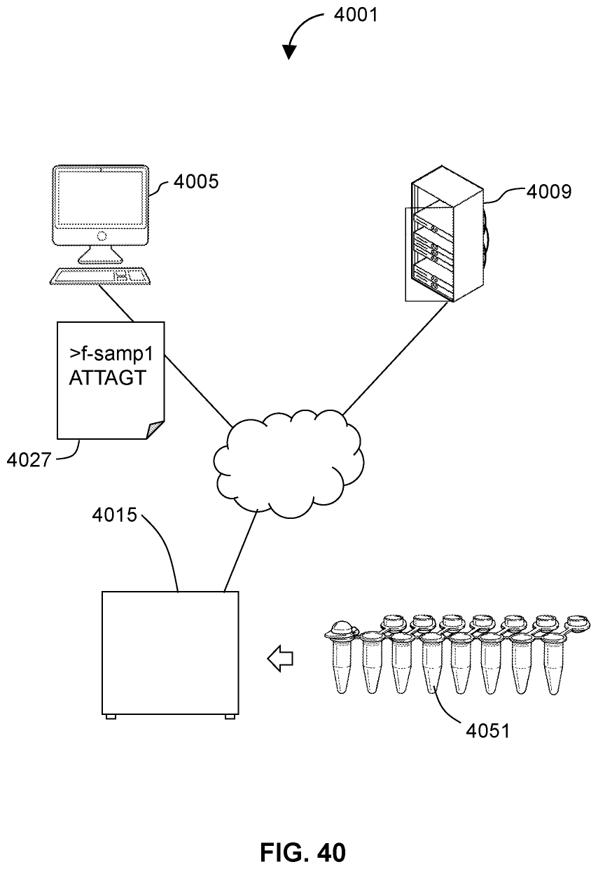

FIG. 40 shows a system 4001 that may be used for performing processes, steps, and methods of the invention. The system 4001 may include at least one sequencing platform 4015 optionally connected to a computer system 4005. Samples may be provided in, and process reactions conducted in, laboratory containers 4051 or other suitable device or compositions (e.g., droplets in microfluidic channels or emulsions). Chemical and/or enzymatic reactions and processes of the invention generate products, e.g., in the containers 4051, that may be sequenced using at least one sequencing platform 4015 to generate a plurality of sequence reads 4027. An analysis unit may include computer program instructions and processing operations provided by the connected computer 4005. Functions of the analysis unit may be distributed among any of computation hardware in the sequencing instrument 4015, remote computing system 4009, such as server or cloud computer or combination thereof, and the computer 4005. For example, read trimming and base quality scoring may be performed within sequencing instrument 4015. Read assembly and or mapping (e.g., to a reference or an atlas) may use the remote computing system 4009. Analysis of modification patterns, e.g., based on base calls in the individual sequence reads 4027, to make modified base calls for the original fragment, may be performed by analysis unit modules in computer 4005, which may be programmed to implement base calls according to the logic and intent of any of processes, workflows, and tables shown herein.

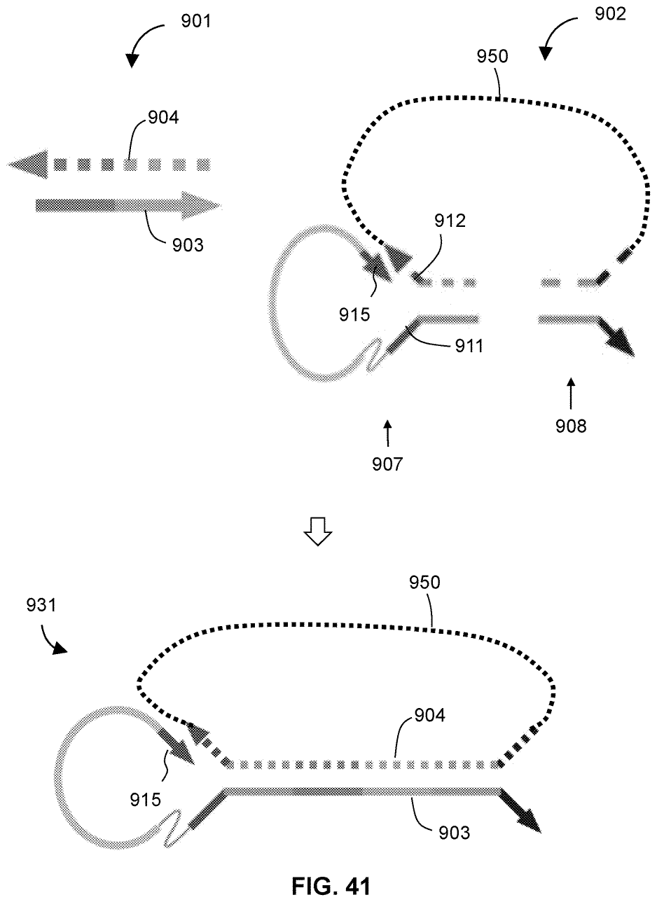

FIG. 41 illustrates an embodiment that uses linked adaptors 902 to make a complex useful in methods herein. As shown, a first Y adaptor portion 907 includes a first oligo 911 linked to a primer 915 that is annealed to a second oligo 912. The linked adaptors include a second Y adaptor portion 908 that is linked to the first Y adaptor portion 907 via a linker 950. The linker 950 may be any suitable molecular structure including a nucleic acid strand, PEG, inverted bases, non-natural bases, spacers, others, or combinations thereof. A fragment 901 of DNA will have the linked adaptors 902 ligated thereto to form a complex 931.

The second strand 904 in the complex 931 appears as a circular structure due to the linker 950. The primer 915 may be extended to make a copy of the second strand. The linker 950 preferably include at least one spacer, bond, molecule, or feature other than a nucleic acid base so that the polymerase stalls and falls off (and does not copy the linker 950). Once the primer 915 is extended, the first strand 903 and the resultant extension products provide a complex with essentially all of the elements of the complex 131 (except that the second strand 904 is in the circularized structure). That complex may be subject to any of the processes, reactions, or workflows shown herein. The linked adaptors 902 provide a particular functionality in that it is probable that the first Y adaptor portion 907 and the second Y adaptor portion 908 will both be ligated to the same fragment 901. Without being bound by any mechanism, it may be that ligation of one of adaptor portion 907, 908 to one end of the fragment 901 brings the other adaptor portion into proximity with the fragment 901. Because the linked adaptors 902 will be ligated to the same fragment, they present an opportunity to put congruent barcodes on both ends of the fragment and/or ensure faithful adaptor ligation for very rare fragments, e.g., in cell-free DNA, for example.

FIG. 41 illustrates an embodiment that uses linked adaptors 902 to make a complex useful in methods herein. As shown, a first Y adaptor portion 907 includes a first oligo 911 linked to a primer 915 that is annealed to a second oligo 912. The linked adaptors include a second Y adaptor portion 908 that is linked to the first Y adaptor portion 907 via a linker 950. The linker 950 may be any suitable molecular structure including a nucleic acid strand, PEG, inverted bases, non-natural bases, spacers, others, or combinations thereof. A fragment 901 of DNA will have the linked adaptors 902 ligated thereto to form a complex 931.

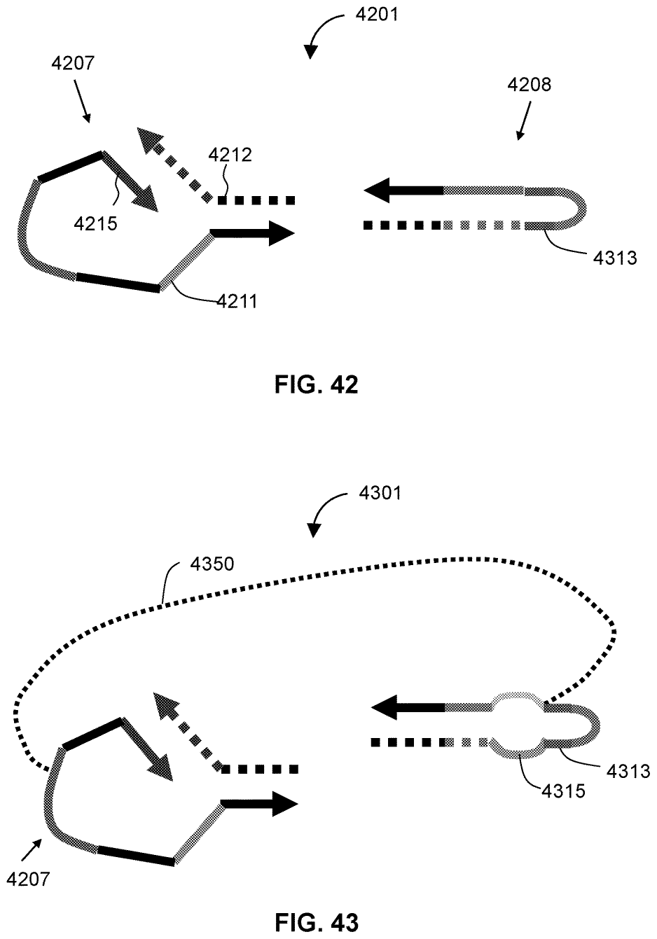

FIG. 42 shows a set of adaptors 4201 that may be used for mPS workflows. The adaptors 4201 and related embodiments create a molecular tool useful in a workflow in which one or more methylation conversion steps may be applied prior to a Pro-Seq extension step (which links the fragments). To allow a step such as TAPS, CAPS, or bisulfite treatment to be used (before strand extension), a hairpin adaptor may be used to keep corresponding sense- and antisense-strands together when input into downstream steps. The adaptors 4201 include a linking adaptor 4207 and a hairpin adaptor 4208. The linking adaptor 4207 includes a first oligo 4211 is linked to a primer 4215 annealed to a second oligo 4212. The hairpin adaptor 4208 may include an optional cleavage site 4313 (e.g., a uracil or a restriction site) The adaptors 4201 may be used in library preparation and/or sequencing methods that include ligating the adaptors 4207, 4208 to opposed ends of a nucleic acid fragment. The first oligo 4211 is covalently ligated to a first (or “sense”) strand of the fragment and the primer 4215 is extended to make an extension product that includes a copy of a second strand of the fragment. The extension could happen before or after any treatment, e.g., CAPS. The hairpin adaptor 4208 is ligated to the first strand and the second strand, keeping those strands together (e.g., in “proximity” with each other) and also together with the copy of the first strand (the copy may be made before after any reannealing). Extending the primer 4215 forms a molecular complex that includes: the first strand ligated to the first oligo 4211, the first oligo 4211 linked to the primer 4215 wherein the primer 4215 is extended into a product that includes a copy of the second strand, and the second strand attached to the first strand through the ligated hairpin adaptor 4208. That complex may include primer binding sites provided by the adaptors 4201. In the complex, the second strand may be in tandem in a continuous segment of nucleic acid with the first stand if the hairpin adaptor 4208 is not cleaved at a cleavage site 4313.

FIG. 43 shows adaptors 4301 with various optional features. Any of the features of adaptors 4301 may be applied to or included in adaptors 4201. The adaptors 4301 may include a linking adaptor 4207 and optionally a hairpin adaptor. The hairpin adaptor may include an optional bubble 4315, allowing downstream polymerase extension products to be provided with strand specific segments (e.g., barcodes, primer-binding sites, etc.). The hairpin adaptor may include an optional cleavage site 4313 (e.g., a uracil or a restriction site) and the adaptors 4301 maybe linked together by an optional linker 4350.

The adaptors 4201 may equally as well be attached together by a linker 4350. A benefit of the linker 4350 is having the adaptors linked together promotes the result that the linked pair of adaptors gets ligated to opposed ends of a nucleic acid fragment. This is useful when unlike or dissimilar adaptors are used. A large number of dissimilar adaptors may be linked together with linkers 4350, which may be useful in whole genome sequencing, particularly for cell-free DNA (cfDNA) and any included ctDNA. The fragments of cfDNA have a high likelihood of being ligated to adaptors 4201 in pairs as shown due to the linker 4350, allowing essentially all fragments of cfDNA to be captured by the depicted adaptors. Thus paired adaptors 4201 linked by linkers 4350 are one tool by which to bridge the gap from the inclusivity of whole genome sequencing to the refined information gathering of proximity sequencing and related duplex sequencing methods. Adaptors (such as a adaptors 4201 or other adaptors shown herein) linked together may be used to prepare cfDNA for Pro-Seq or duplex sequencing by capturing essentially all fragments from a sample such as a blood or plasma sample obtained by liquid biopsy. Another benefit of the linker 4350 is that the second strand and first strand of a fragment will be linked at their 5′ ends so even though those molecules may be complementary, the order of one is “reversed” in a way that minimizes re-annealing. Any suitable molecule or structure may be used as a linker 4350 including an oligonucleotide, a solid support (e.g., a bead or flow cell), other linkers such as PEG (e.g., units of BM-PEG 25 or similar), or combinations thereof.

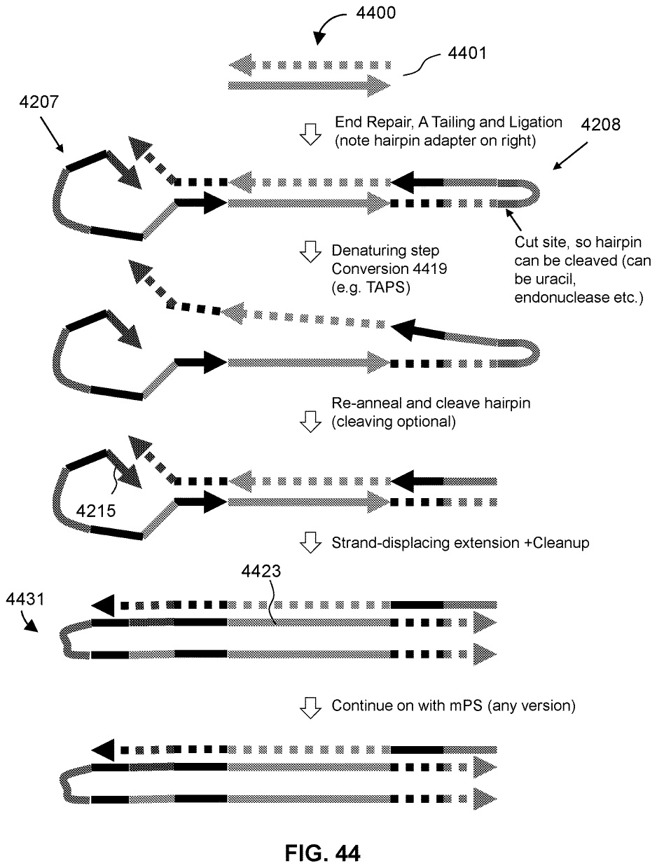

FIG. 44 illustrates an mPS process 4400 using the adaptors 4201. The process 4400 preferably includes ligating linking adaptor 4207 and hairpin adaptor 4208 to a nucleic acid fragment 4401 (after any optional end-repair and/or A-tailing steps). The hairpin adaptor 4208 will connect and hold together both complementary strands of the fragment 4401. Due to this, the process 4400 may include a conversion 4419 or other step prior to the Pro-Seq extension step (which links the fragments). As shown, after adaptor ligation, the process may include TET-assisted pyridine borane sequencing (TAPS), which uses tet methylcytosine dioxygenase 1 (TET1) to oxidize all the 5mC and 5hmC into 5-carboxycytosine (5caC). The 5caCs may then be reduced to dihydrouracil (DHU) using pyridine borane. Subsequent PCR would convert DHU to thymine, enabling cytosines and the corresponding cytosine modifications to be differentiated. After any denaturing and conversion 4419 (e.g., TAPS), the strands may be re-annealed and the hairpin may be cleaved. As shown, the process 4400 may include extending the primer 4215 using a strand-displacing polymerase.

This produces a molecular complex 4431 that includes: a first strand of the fragment 4401 linked to primer 4215 now extended into a product that includes a copy 4423 of the second strand, with the second strand annealed to its copy 4423 in the product. In the process 4400, the complex 4431 may be used as an input into versions of the mPS workflows shown herein.

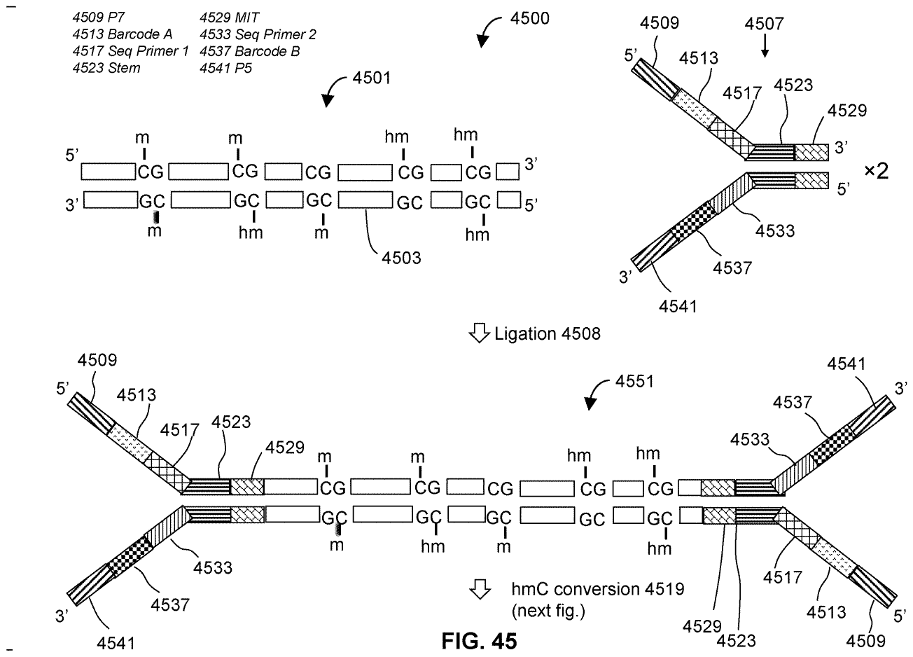

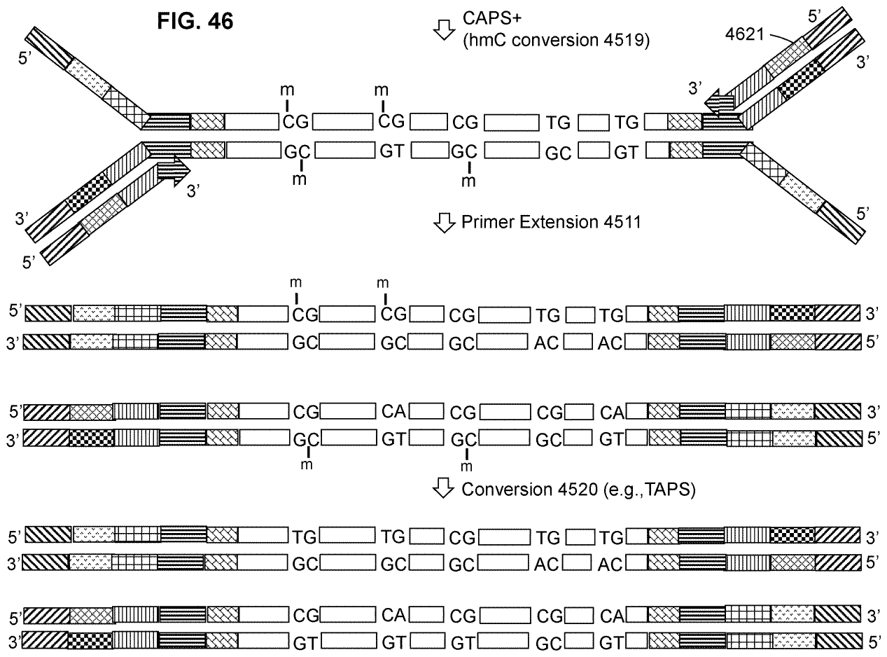

Other embodiments of methylation Proximity Sequencing (mPS) are provided by the disclosure. FIG. 45 and FIG. 46 show an approach to mPS that uses Y-adaptors in ‘6-base’ sequencing using methyl and hydroxy CAPS-TAPS Seq technologies.

FIG. 45 shows a fragment 4501 and two adaptors 4507 used in a methyl and hydroxy CAPS-TAPS Seq process 4500. The process 4500 like others herein is suited to genomic sequencing technologies such as whole-genome sequencing WGS including WGS from blood or plasma samples, e.g., as obtained by liquid biopsy and sometimes described as plasma WGS (pWGS). The process 4500 may include ligating 4508 adapters 4507 to one or more fragment(s) 4501 to yield one or more adapter-ligated fragment(s) 4551. As shown, an adapter-ligated fragment 4551 includes a P7 primer capture sequence 4509, Barcode A 4513, Seq Primer 16317, a Stem region 4523, a molecular identifier tag (MIT) 4529, Seq Primer 2 4533, Barcode B 4537, and a P5 primer capture sequence 4541. The MIT may be a unique molecular identifier (UMI) and may be unique among a set of a adaptors or nearly unique (e.g., randomized bases with enough dissimilarity that no two identical fragments are likely to be ligated to identical MITs.

After ligation produces an adapter-ligated fragment 4551, the adapter-ligated fragment 4551 is preferably subject to a process for hmC conversion 4519 such as chemical-assisted pyridine borane sequencing (CAPS) or CAPS+. CAPS+is described in Xu, 2023, Modular oxidation of cytosine modifications and their application in direct and quantitative sequencing of 5-Hydroxymethylcytosine, J Am Chem Soc 145(13):7095-7100, incorporated by reference.

The pre-treatment adaptor-ligated fragment 4551 is shown. As shown, the top (or “forward” or “sense”) strand includes, from 5′ to 3′: mC, mC, C, hmC, hmC. The bottom (or “reverse” or “antisense”) strand includes, from 5′ to 3′: hmC, C, mC, hmC, and mC. In the process 4500, the adaptor ligated fragment is preferably treated (e.g., by CAPS or CAPS+) to convert (i.e., hmC conversion 4519) hmC to DHU, which will be read as (and drawn as) T.

FIG. 46 shows a CAPS step of the process 4500 and a resulting product. Notice that the product has single-stranded, or “forked”, ends because the adaptors 4507 are preferably Y-adaptors that include a double-stranded stem segment (available for ligation to ends of a fragment 4501 (e.g., after any optional blunt-ending, A-tailing, etc.). The hmC conversion 4519 (e.g., CAPS treatment) removes hydroxymethyl groups, leaving those to be read as Tin downstream steps.

In the process 4500, after hmC to DHU conversion (e.g., CAPS), primers 4621 are annealed to the product and extended at a primer extension step 4511.

As shown, extending 4511 primers creates at least two double-stranded products. Note from the drawings that primer extension incorporates: a C based-paired where the CAPS-treated adapter-ligated fragment 4551 included a G; a G based-paired where fragment 4551 included mC or C; and a T base-paired where fragment 4551 included hmC.

After the primer extension 4511, the products are subject to an conversion 4520 such as TET-assisted pyridine borane sequencing (TAPS) conversion. In TAPS, all 5mC and 5hmC are converted to DHU, for which PCR incorporates T. Notably, after TAPS conversion, there are four strands that carry sequence information of the original fragment 4501. Among the four strands, each variant of C (C, mC, and hmC) are represented differently.

Those four strands may be subject to amplification and sequencing. For example, because the end may include primer captures sites such as P5/P7 primer binding sites, the strands maybe captured to a solid support such as a flow cell or a bead and amplified to form amplicons or a cluster (or amplicons that are captured to a support and further amplified to form clusters). The amplicons or cluster(s) may be subject to a sequencing reaction in a sequencing instrument such as from ILLUMINA or ULTIMA.

Sequences with Barcode A and Barcode B have methylation and hydroxymethylated residues converted. Sequences with Barcode A and Barcode C have only hydroxymethylated residues converted. MITs can be used to informatically link together sequence reads from each starting fragment 4501, to thereby provide both methyl and hydroxymethyl information for each single input molecule.

The process 4500 preferably begins with ligating 4508 adaptors 4507 to fragments 4501 from a sample, and provides information about epigenetic modifications in those fragments 4501.

Systems and methods of the invention are useful for determining sequences of, and patterns of genetic and epigenetic modifications in, genetic material. Methods of the invention use proximity sequencing, in which two polynucleotides that are independently obtained from fragment of DNA are linked together, keeping those polynucleotides together for chemical or enzymatic treatment, amplification to prepare a sequencing library, and sequencing. Due to the chemical or enzymatic treatment, different base modifications give different base calls after sequencing, and by applying the logic of what base calls would be expected, methods of the invention may provide a series of “base calls” including the epigenetic modifications for the fragment of DNA. Methods of the invention may be used not only to determine sequences and epigenetic markers, methods may further include the detecting, reporting, or use of phased variants, which include two or more mutations (relative to a reference) that appear within a limited distance of each other on one strand of DNA from a sample. The distance may be limited to less than about 200 bases, e.g., less than about 170 bases, so that that a phased variant biomarker is one that may be found among cell-free nucleic acid (which may have a mean or modal fragment length of about 170 bases). When a sample nucleic acid is analyzed to detect a phased variant that is present in a strand of the sample nucleic acid, it is highly improbable that a sample preparation artefacts such as polymerase error will generate an artefact that matches the phased variant. Additionally, methods of the invention use sample preparation techniques that are robust against false positives due to artefacts. The nucleic acid to be analyzed may be analyzed using (i) proximity sequencing (Pro-Seq), in which a fragment is copied redundantly using linked primers to make linked copies and in a manner such that an error introduced during copying does not appear in all copies, and/or (ii) linked target capture (LTC), whereby a nucleic acid is amplified by primers that are linked to target-specific probes to provide highly target-specific enrichment, providing for redundant copes of the target and highly sensitive detection.

Methods of the invention have particular applications in cancer detection and treatment. For example, a tumor sample may be assayed to discover tumor-specific “tumor markers” including sequences, epigenetic markers, and/or phased variants. Inherent to Pro-Seq is that true tumor markers from the tumor genome are exquisitely sensitive and specific for the tumor, while an artefact such as a polymerase error will only appear in a predictable proportion of sequence reads and may be identified as an artefact. Not only do methods of the invention have utility in discovering tumor markers, methods of the invention are particularly well-suited for the detection of such a tumor marker, after it has been discovered, later in a patient's journey. For example, after a patient has been treated to remove a tumor, it may be clinically important to later detect any evidence of the tumor in the patient. That is, it may be important to detect any minimal residual disease (MRD). Sample preparation methods of the invention are well suited for the detection of tumor markers as evidence of MRD due to their specificity and robustness.

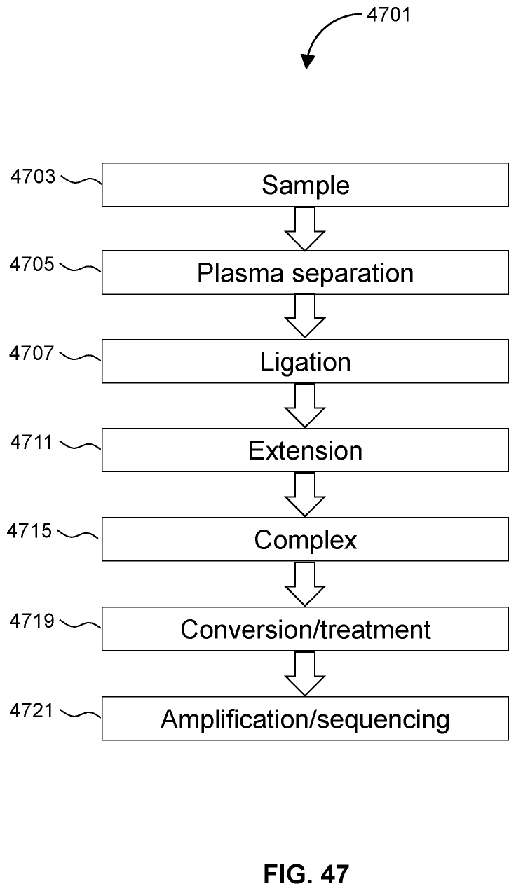

FIG. 47 diagrams a genome sequencing process 4701. The process 4701 is useful for sequencing genetic material from a subject using adaptors, workflows, methods, and reagents from this disclosure. This process 4701 may be used for sequencing genetic material in a manner referred to as whole genome sequencing (WGS) as recognized in the art. Art-recognized WGS techniques do not require the sequencing of a whole genome. Rather the terminology is used to distinguish from targeted sequencing, e.g., using gene-specific or target-specific primers for initial target capture. The process 4701 involves the use of proximity sequencing (Pro-Seq) with methylation profiling in plasma WGS (pWGS). In Pro-Seq, at least two strands carrying nominally similar sequences bases (the same but for any lesions (e.g., abasic sites or thymine dimers) or polymerase errors) are linked to one another to be held in proximity to each other. In Pro-Seq in the process 4701 the at least two strands are or are from a sense strand a complementary antisense strand that formed a duplex in a sample.

The process 4701 includes obtaining 4703 a sample. For example, a sample may be a blood draw collected into a suitable tube such as a blood collection tube (e.g., 10 mL) or other suitable sample. Blood may be collected using a cell-stabilizing BCT tube (Streck, La Vista, NE, USA) at a suitable clinical facility and shipped to and received at a facility performing the process 4701.

The process 4701 preferably includes separating 4705 plasma from cells by a suitable technique. For example, cell-free plasma and white blood cells (WBC) may be separated by centrifugation (e.g., of the blood for 10 minutes at 1,700 xg followed by 10 minutes at 20,000 xg, optionally stored at −80 ° C until further processing). Cell-free DNA (cfDNA) may be isolated from the plasma using suitable reagent and equipment. For example, embodiments use the circulating DNA kit sold under the trademark QIAsymphony DSP Circulating DNA Kit (QIAGEN, Hilden, Germany).

The process 4701 may include ligating 4707 adaptors to DNA fragments in a first step of WGS library preparation. Adaptor ligation 4707 may optionally include one or more of end-repair, A-tailing, and adapter ligation with any adaptors or combination of adaptors of the present disclosure. For example, certain preferred embodiments include ligating 4707 a linking adaptor 107 onto at least one end of a fragment. Depending on application and embodiment, a fragment may be ligated with: two of linking adaptor 107 (FIG. 2), linking adaptor 107 and Y-adaptor 108 (FIG. 1); a linking adaptor 907 linked to a Y-adaptor 908 (FIG. 41); a linking adaptor 1507 and a double-stranded second adaptor 1508 (FIG. 20); a linking adaptor 1007 and a bubble adaptor 1008 (FIG. 13); a linking adaptor 4207 and a hairpin adaptor 4208 (FIG. 42); a linking adaptor 4207 linked to a hairpin adaptor 4208 (FIG. 43); or two Y-adaptors (FIG. 45). As shown variously herein, the adaptor(s) ligated 4707 to the fragment(s) may include primer binding sites or other features such as a molecular identification tag (MIT), a unique molecular identifier (UMI), a sample barcode, an indexing barcode, or other custom barcodes.

After ligation 4707 of any adaptor(s) to fragment(s), the process 4701 may include extending 4711 a primer (e.g., of a linking adaptor) preferably using strand-displacing polymerase to provide 4715 a complex such as complex 131, 231, 431, 1031, 1531, 2631, 3031, 4431.

The process 4701 includes subjecting the complex to a conversion 4719 or treatment step in which the treatments and enzymes of any of the methyl proximity sequencing (mPS) embodiments herein are performed to convert bases of the complex. It is noted that providing 3715 a complex is a crossover point or break point and the process 4701 may terminate here, and a converted/treated complex after conversion step 4719 may be stored (e.g., in a −80 degree freezer) or shipped for analysis. Similarly, the process 4701 may may begin with the conversion step 4719 if a separate party has performed plasma separation 4705 and adaptor ligation 4707 steps. The conversion or treatment 4719 is performed according one of the workflows shown in the disclosure and provides a treated sequencing library, obtained from plasma, and suitable for WGS, i.e., for plasma WGS (pWGS).

The library may optionally be amplified 4721 (e.g., PCR and/or on a substrate such as by bridge amplification) and optionally sequenced on a suitable sequencing platform. Suitable sequencing platforms include the Illumina NovaSeq6000 platform (Illumina, USA) as well as sequencing platforms from Ultima Genomics, Oxford Nanopore, Pacific Biosciences, and others. For example, in certain embodiments, the library is sequenced on ILLUMINA to generate sequence data (e.g., sequence reads). Sequence data may be demultiplexed and optionally de-duplicated (sometimes called “collapsing sequencing reads”) using MITs or UMIs. FASTQ files may be quality trimmed using e.g. Trimmomatic (v0.33) and optionally aligned to a reference (e.g., the hg19 human reference genome) using suitable software (e.g., BWA-MEM2) for variant identification.

In certain embodiments of the process 4701, tumor-specific variants have previously been discovered by sequencing tumor DNA from a known tumor sample such as formalin-fixed, paraffin embedded (FFPE) slice from a tumor or from fresh frozen DNA from a tumor biopsy. The tumor DNA may be treated by mPS to detect epigenetic patterns. Sequencing known tumor DNA to obtain tumor sequences that are mapped to a reference or to “matched normal” tumor sequences is used to discover tumor specific variants. The epigenetic patterns of the tumor DNA may also be part of the set of tumor-specific variants. Information describing the tumor-specific variants, which includes patterns and positions of C, mC, and hmC, is stored and the process 4701 may include pWGS to detect those tumor-specific variants later in a liquid biopsy sample.

### Examples

### Example 1: mPS Demonstration Data

Methods were performed to determine patterns of epigenetic modifications in DNA fragments harboring mC using a Pro-Seq complex (i.e., methylation Pro-Seq or mPS), in which two polynucleotide strands that are independently obtained from a fragment of DNA are linked together with a linking adaptor 107. The linked strands were treated so that 5mC was identified when the strands are sequenced. The methods. The Pro-Seq complex was treated with TET-assisted pyridine borane sequencing (TAPS)

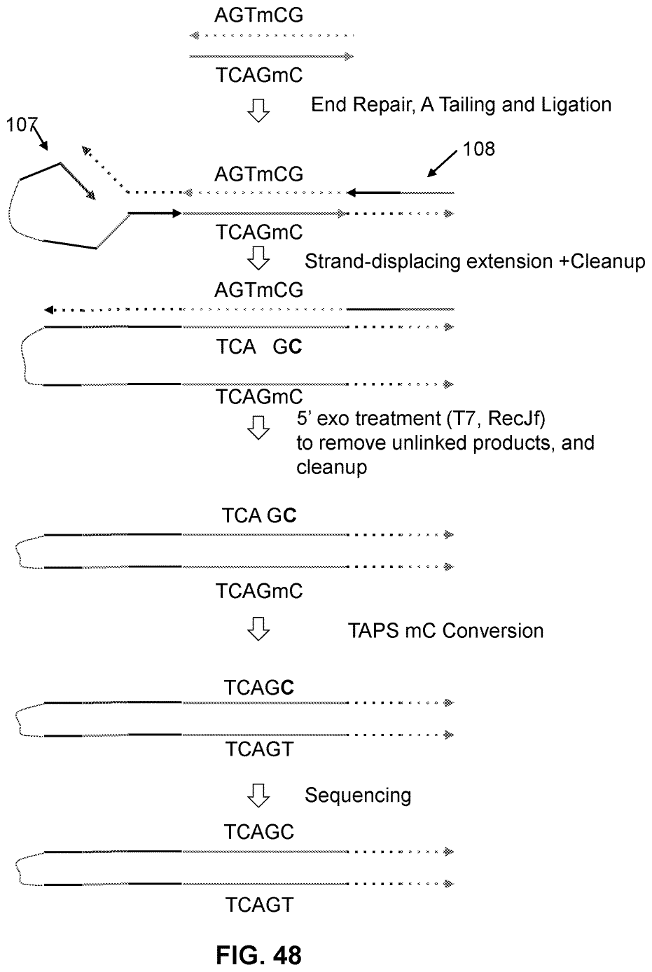

FIG. 48 shows steps in this mPS demonstration with a fragment comprising a forward strand and a complementary reverse strand as drawn here to illustrate the steps. The forward strand included 5′-TCAGmC-3′ and the reverse strand included 5′-G mC TGA-3′.

The process included End Repair, A Tailing and Ligation to attach adaptors 107, 108 FIG. 6 illustrates seeding a flow cell for cluster generation. Bound fragments are copied by flow cell primers, releasing the linked complex, cluster generation occurred as discussed elsewhere herein.

FIG. 7 shows a cluster.

FIG. 49 illustrates how base calls will be made after sequencing to generate Read 1 and Read 2. The base calls are shown in the adjacent consensus sequence. As oriented on the page, the bottom base was read as T in Read 1 and read as Cin Read 2, and called as mC for the fragment. An unmodified C in the fragment will appear as C in both reads and can be called as such. The epigenetic modifications and their positions may be identified by software, e.g., a software module that is written according to the logical shown here and executed by computer system that analyzes sequence data.

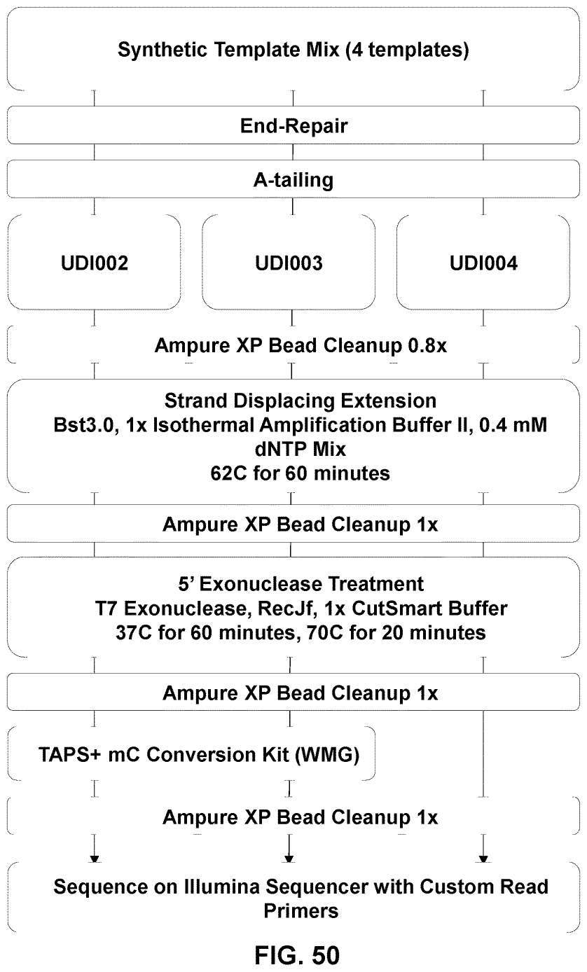

FIG. 50 gives a diagram of a workflow used in this Example 1 using TAPS+for four template fragments. The four templates—labeled mPS t1, mPS t2, mPS t3, and mPS t4 are described here.

Example 1 used a first template (mPS t1). The first Synthetic Template (mPS t1) is blunt at one end, has 8-base 5′ overhang, and includes about 15% methylation at CpGs

The sequence of a bottom, or sense, strand of mPS t1 is:

(SEQ ID NO: 1)
5′-TTGAGCGCGT AGGCTTCGAT ATCGTCGGTC GGGTCATACG
TGGACTTGTC AcGCTTGCTC CACTCCGTGC CGCCGGACTA
TTCTCCTCAC TGcGGCCCAT ATCCACCTCA ACCGGATCGA
AGGCTTCACC GGTCATGGTG-3′

In that sense strand of mPS t1, positions 52 and 93 (marked by a lowercase C) are 5mC.

The sequence of a top, or antisense, strand of mPS t1 is:

(SEQ ID NO: 2)
5′-CACCATGACC GGTGAAGCCT TCGATCCGGT TGAGGTGGAT
ATGGGCcGCA GTGAGGAGAA TAGTCCGGCG GCACGGAGTG
GAGCAAGcGT GACAAGTCCA CGTATGACCC GACCGACGAT
ATCGAAGCCTAC-3′

In that antisense strand of mPS t1, positions 47 and 88 are 5mC.

Example 1 used a second template (mPS t2). The second template (mPS t2) has an 8-base 3′ overhang, a 16-base 5′ overhang, and 50% methylation (5/10). The sequence of a bottom, or sense, strand of mPS t2 is:

(SEQ ID NO: 3)
5′-ACGGACTTTG CCCGCCTGCA AGCTGcGTGG CCACGTCGGT
GAACTGTGCA GGCAGCATAc GCATGGCGGC TTTATACTGC
TCCCcGCTTT CTGTGCAGCC AGCGCCTGTc GGCTCAGcGA
CTGTTCAACG ACTGCCGCTG-3′

In the sense strand of mPS t2, positions 26, 60, 85, 110, and 118 are 5mC.

The sequence of a top, or antisense, strand of mPs t2 is:

(SEQ ID NO: 4)
5′-GTCGTTGAAC AGTcGCTGAG CcGACAGGCG CTGGCTGCAC
AGAAAGcGGG GAGCAGTATA AAGCCGCCAT GcGTATGCTG
CCTGCACAGT TCACCGACGT GGCCAcGCAG CTTGCA-3′

In the antisense strand of mPS t2, positions 14, 22, 47, 72, and 106 are 5mC.

Example 1 used a third template (mPS t3). The third template (mPS t3) has a 16-base 3′ overhang and one blunt end with 85% methylation (11/13).

The sequence of a bottom, or sense, strand of mPS t3 is:

(SEQ ID NO: 5)
5′-CGCTCAGACT GCCCCcGTGT TATTcGTCGA TTTGGTGCcG
TAATcGAAAC TGCCTGTTGT TTTcGTCCcG TAATCAAAcG
AcGATGTGGT TTTCGTCCCC AAATCcGTAC cGGATGCACT
GGCACTGTGG GTGTGCGACT-3′

In the sense strand of mPS t3, positions 16, 25, 28, 39, 45, 64, 79, 82, 108, and 111 are 5mC.

The sequence of a top, or antisense, strand of mPs t3 is:

(SEQ ID NO: 6)
5′-TGCCAGTGCA TCcGGTACGG ATTTGGGGAC GAAAACCACA
TcGTcGTTTG ATTAcGGGAc GAAAACAACA GGCAGTTTcG
ATTAcGGCAC CAAATcGAcG AATAACAcGG GGGCAGTCTG
AGCG-3′

In the antisense strand of mPS t3, positions 13, 18, 42, 45, 55, 59, 7985, 97, 99, and 109 (lowercase c) are 5mC.

Example 1 used a fourth template (mPS t4). The fourth template, mPS t4, was blunt at both ends and included 0% methylation (e.g., 0/12 C positions methylated)

The sequence of a bottom, or sense, strand of mPS t4 is:

(SEQ ID NO: 7)
5′-GCCAACAACA TGCCGGAACA GTACGACGGG TACAGCAGGT
AGCGCAGATC ATCAACGGTG TGTTCAGCCA GTTACTGGCA
ACTTTCCCGG CGAGCCTGGC TAACCGTGAC CAGAACGAAG
TGAACGAAAT CCGTCGCCAG-3′

The sequence of a top, or antisense, strand of mPs t4 is:

(SEQ ID NO: 8)
5′-CTGGCGACGG ATTTCGTTCA CTTCGTTCTG GTCACGGTTA
GCCAGGCTCG CCGGGAAAGT TGCCAGTAAC TGGCTGAACA
CACCGTTGAT GATCTGCGCT ACCTGCTGTA CCCGTCGTAC
TGTTCCGGCA TGTTGTTGGC-3′

The workflow included providing a Synthetic Template Mix (4 templates), performing end-repair, A-tailing, and adaptor ligation to provide fragments labeled with unique dual index 1 (UDI001), UDI002, and UDI003.

The workflow included Ampure XP bead cleanup (at 0.8×) followed by strand displacing extension, which produces a complex as described herein. Extension used Bst3.0 in 1× Isothermal Amplification Buffer II with 0.4 mM dNTP mix, incubated 62 degrees C. for 60 minutes followed by Ampure XP Bead Cleanup (1×).

The workflow further included 5′ Exonuclease Treatment using T7 Exonuclease and RecJf in 1× CutSmart Buffer at 37 C for 60 minutes the 70C for 20 minutes followed by bead cleanup.

Then, complexes labeled UDI001 and UDI002 were subject to TAPS mC conversion using a TAPS+kit from Watchmaker Genomics, followed by bead cleanup (1×)

The resulting complexes were sequenced on an Illumina Sequencer with Custom Read Primers according to sequencing methods and read analysis/base-calling techniques of the present disclosure.

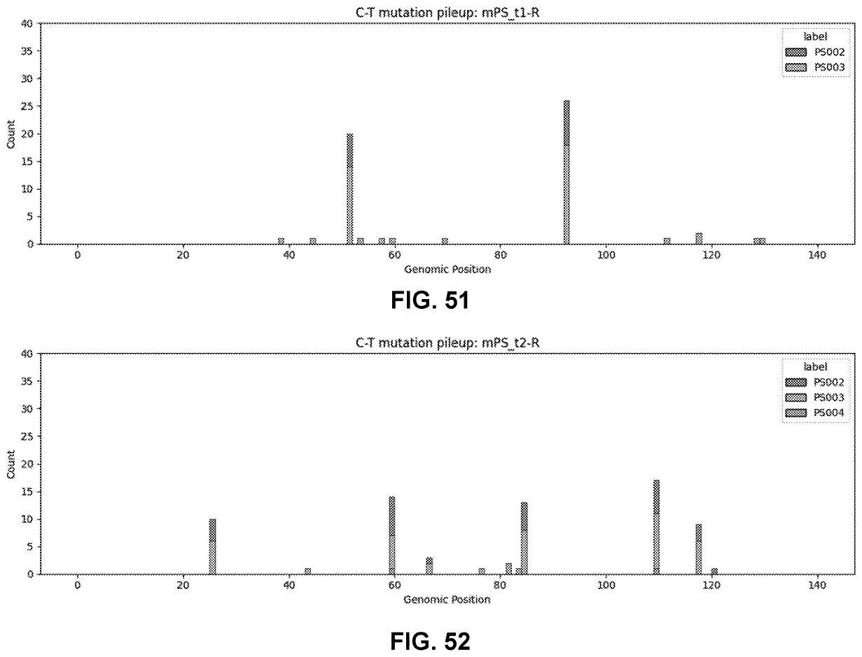

FIG. 51 shows sequence data from mPS t1. Using consensus logic shown in FIG. 49, the plot show positions in mPS t1 with a C in strand 1, and a T in strand 2. The expected methylation positions are observed above background. Background noise is negligible (easily excludable by software) and likely to be associated with synthesis errors (synthetic template) and/or sequencing noise.

The data show the detection of 5mC at positions 47 and 88.

FIG. 52 shows sequence data from mPS t2. Similar Plots for mPS t2 were obtained as for mPS t1. PS004 did not have TAPS methylation conversion, and no signal is observed, as expected. The data show the detection of 5mC at positions 26, 60, 85, 110, and 118.

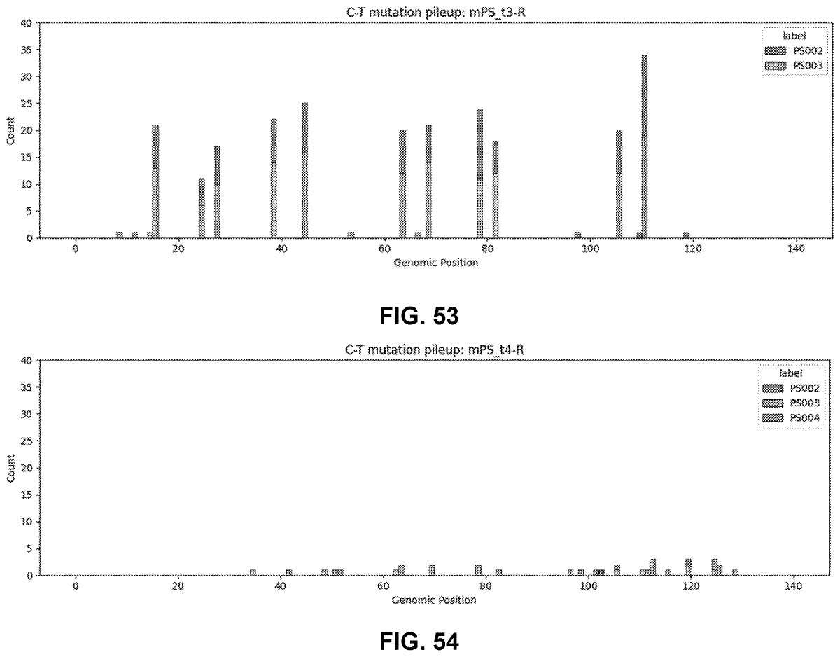

FIG. 53 shows sequence data from mPS t3. As shown, 5mC was detected at positions 16, 25, 28, 39, 45, 64, 79, 82, 108, and 111.

FIG. 54 shows sequence data from mPS t4. As shown and expected, the for mPS t4 which included no methylation, no mPS signal is present among the data.

The data of Example 1 show that mPS according to the disclosure is useful to reliably detect 5mC in DNA fragments.

### Example 2: HmPS Demonstration Data, CAPS & TAPS Method

Nucleic acid fragments that included hydroxymethyl cytosine (5hmC or hmC) as well as 5mC) were subject to Pro-Seq, conversion by chemical-assisted pyridine borane sequencing (CAPS) and by TET-assisted pyridine borane sequencing (TAPS) to detect and determine positions of hmC.

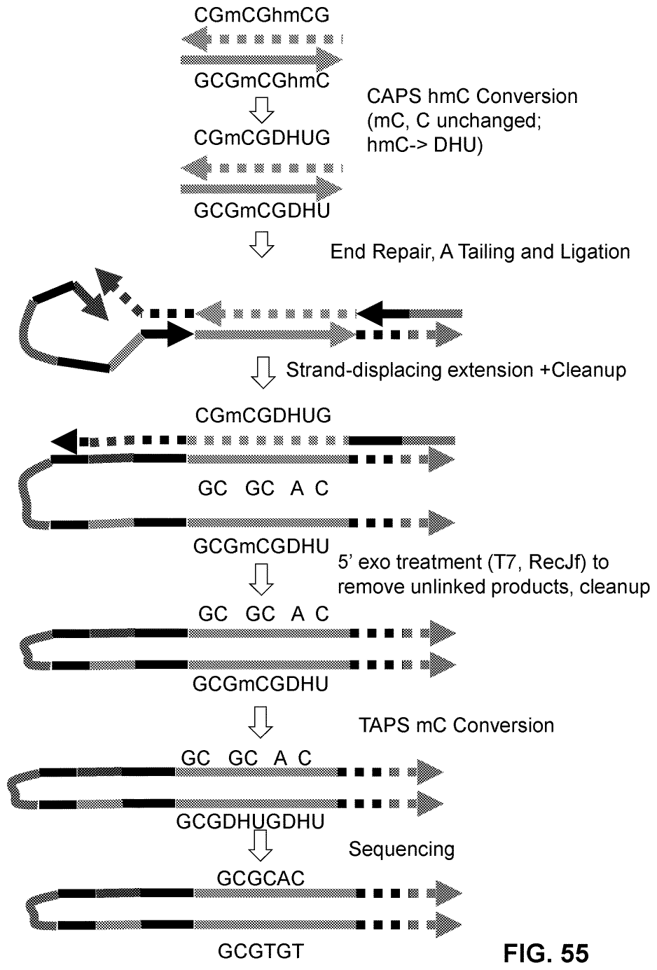

FIG. 55 is a diagram of the workflow. For a fragment, a forward (or “bottom”) strand is drawn showing it to include 5′-GCGmCGhmC-3′ while a complementary reverse (or “top”) strand includes 5′-GhmCmGmCGC-3′, as drawn.

The steps include CAPS hmC conversion (mC are C are unchanged while hmC is converted to DHU). The workflow includes end repair, A-Tailing and ligation of a linking adaptor to yield the depicted complex, followed by strand-displacing extension and cleanup. Then 5′ exonuclease treatment (T7, RecJf) is performed to remove unlinked products with cleanup. The resulting product is subject to TAPS mC Conversion (any mC becomes DHU), and the product is sequenced.

As with before, FIG. 6 illustrates seeding a flow cell for cluster generation. Bound fragments are copied by flow cell primers, releasing the linked complex, and cluster generation occurs as usual. FIG. 7 shows a cluster.

FIG. 56 illustrates a Read 1 and Read 2 that may be generated and a consensus sequence that may be called in software. To generate the consensus, information for the adjacent, preceding base is used. This shows how six-bases sequencing may use contextual information from the strand(s). For example, (as shown) to call a position as hmC, the preceding position in the two reads had to be G and A. That is, where Read 1 has G then T and Read 2 has A then C, the 1/2 T/C position is “called” as hmC for the first strand. Where read 1 has GT and Read 2 has GC in homologous positions, the Read 1 T and Read 2 C position is “called” as mC for strand 1.

Similarly, GC and GC in the two reads supports a “C” call in the consensus, which represents the six-base sequencing call for the first strand. The positions called for the first strand may be implemented in software, e.g., a software module in an analysis unit of, or coupled to, a sequencing instrument.

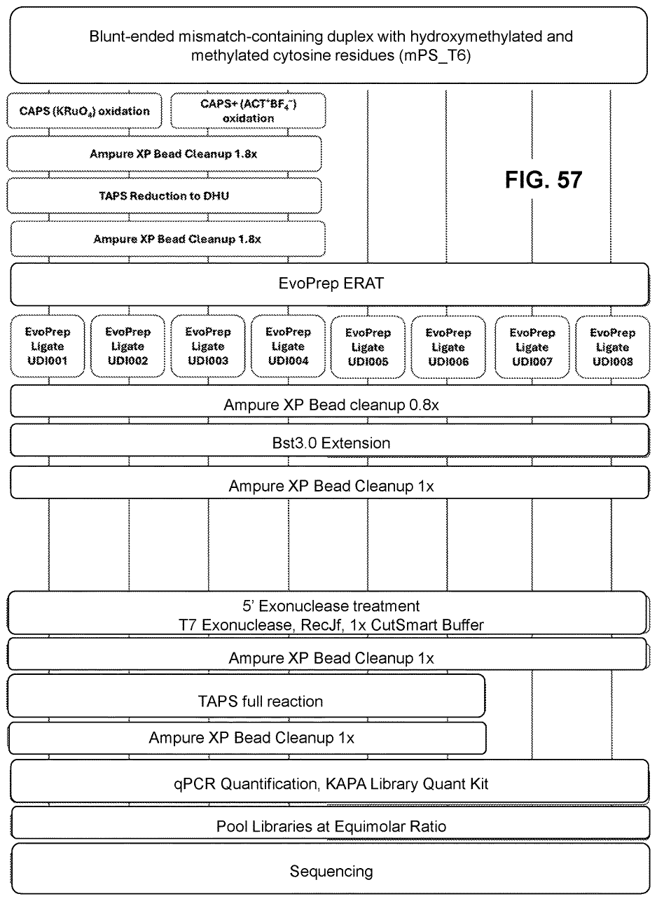

FIG. 57 gives an overview of the process workflow. The steps are labeled consistently as with before. This figure shows the DNMT workflow including treatment of 2 templates with CAPS and another two with CAPS+ and other steps as shown. This Example 1 uses a sixth template (mPS t6). The sixth template, mPS t6, was blunt at both ends and included (among others) five CpG dinucleotides in which the pairs C bases within the five respective CpG dinucleotide were prepared as follows:
1 mC/mC, 1 mC/C, 1 hmC/mC, 1 C/hmC, 1 hmC/hmC

The sequence of a bottom, or sense, strand of mPS t6 is:

(SEQ ID NO: 9)
5′-GCCAGCAGCG CcGGGATTTG TGGAAGGcGG AGAGTCAGTT
CGcGGTACTG GAGGAGGCGG CGCAAcGTCG CCAGCTGTC-3′

In the sense strand of mPS t6, positions 12 and 28 (marked with lowercase c) are 5mC. Positions 43 and 66 (marked with an underlined lowercase c) are 5hmC.

The sequence of a top, or antisense, strand of mPS t6 is:

(SEQ ID NO: 10)
5′-GACAGCTGGC GAcGTTGCGC cGCCTCCTCC AGTACcGCGA
ACTGACTCTC CGCCTTCCAC AAATCCcGGC GCTGCTGGC-3′

In the antisense strand of mPS t6, positions 36 and 67 are 5mC while positions 13 and 21 are 5hmC. Where position 13 in the antisense strand is 5hmC, corresponding position 58 in the sense strand is C, so position 58 of the sense strand is hemi-methylated.

Eight samples were analyzed. Samples 1 and 2 and were analyzed using CAPS & TAPS ‘6-base’ Pro-Seq for sequencing. Samples 3 and 4 were analyzed using CAPS+& TAPS ‘6-base’ Pro-Seq sequencing (the same as for Samples 1 and 2, but the CAPS+kit was used instead of CAPS, expected to have the same results but with higher quality). Samples 5 and 6 were analyzed using TAPS ‘5-base’ Pro-Seq (mPS) while samples 7 and 8 were analyzed using standard ‘4-base’ Pro-Seq. The unique dual index (UDI) numbers in FIG. 57 match the sample numbers, so Sample 1 is labeled with UDI001, Sample 2 is UDI002, and so on.

### Expected Results:

Samples 1-4 Should distinguish variants at positions 12, 28 (mC) from 43 and 66 (hmC). Samples 5-6 Should identify variants at positions 11,27,42 and 46, but miss 58. Samples 7-8 should identify no variants

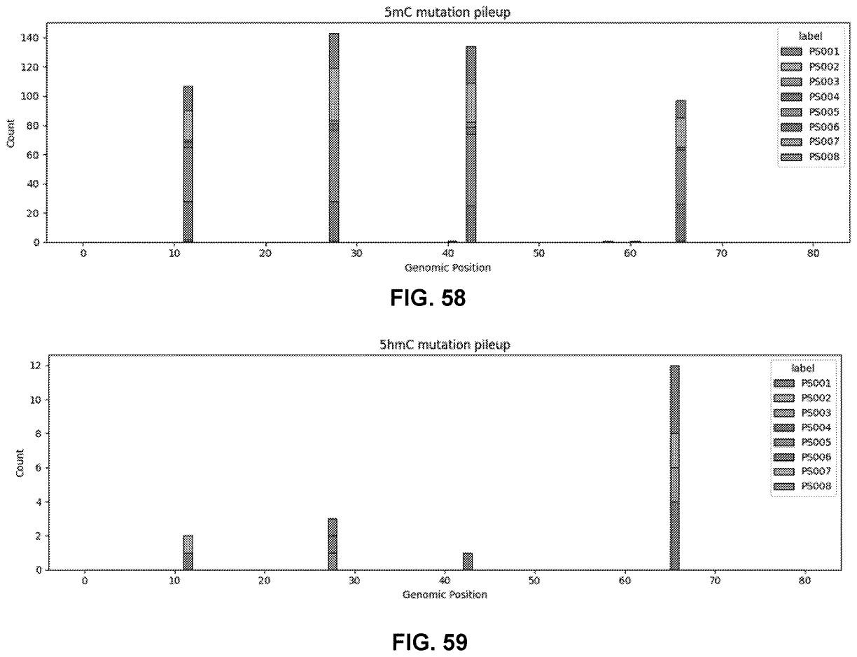

FIG. 58 is a plot showing mC detection in template mPS t6.

Using mC consensus logic shown on FIG. 53 (C: T/G: G), the plot shows mC positions in mPS t6. As expected, detection at positions 12, 28 and 43 is achieved by Samples 1-6 (Note, samples 3-4 were likely underloaded on the sequencer and are thus under-represented in the data). Samples 5-6 also detect mC at position 66 as expected. Some detection by samples 1-4 is also seen at position 66. This could be due to incomplete CAPS conversion. CAPS can be optimized towards complete conversion.

FIG. 59 is a plot showing hmC detection. Using hmC consensus logic shown FIG. 56 (C: T/A: G), the plot shows hmC positions in mPS t6. As expected, detection at position 66 is achieved by Samples 1-4. Other sporadic calls may be due to sequencing and/or synthesis errors (no error correction was applied) and/or non-specific TAPS conversion. TAPS can be optimized to reduce non-specific conversion.

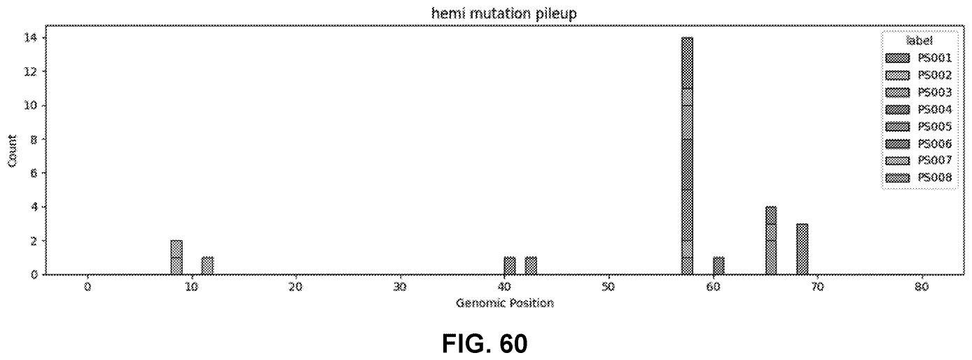

FIG. 60 is a plot showing hemi-hmC Detection. Using hemi-hmC consensus logic (C: C/A: G), the plot shows hemi-hmC positions in mPS t6. As expected, detection at position 58 is achieved by Samples 1-4. Other sporadic calls may be due to sequencing and/or synthesis errors (no error correction was applied) and/or non-specific TAPS conversion.

The plots of FIG. 58 through FIG. 60 and the consensus calling logic are programmed into software in a straightforward manner. These data how that a software module can call C, 5mC, 5hmC, hemi-5mC, and hemi-5hmC positions in a duplex fragment when mPS is used.

### Example 3: HmPS Demonstration Data-DNMT & TAPS Method

Experiments were performed to demonstrate that hydroxy methylation may be identified and located using Pro-Seq (hmPS) using techniques of the disclosure. The experiments use DNA methyltransferase (DNMT) enzymes (which methylate C to 5mC when the C is present in double-stranded DNA, within a hemi-methylated CpG) and TET-assisted pyridine borane sequencing (TAPS).

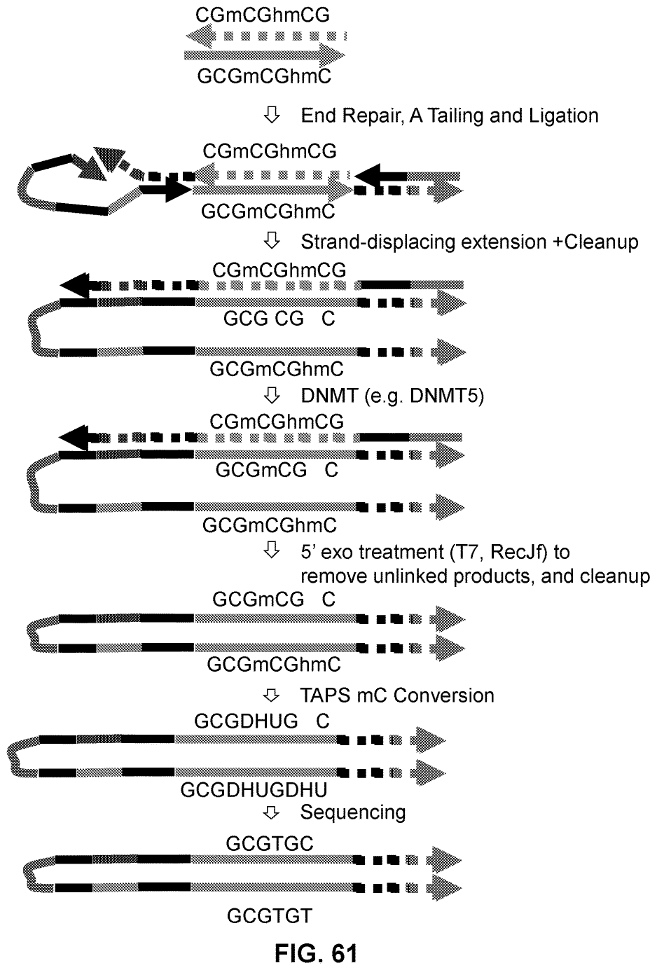

FIG. 61 diagrams a workflow by which hmPS demonstration data were obtained using DNMT & TAPS Methods. The drawing shows a forward (or “bottom” or “sense”) strand of a fragment that includes 5′-GCGmCGhmC-3′ and a reverse (or “top” or “antisense”) strand of the fragment includes 5′-GhmCGmCGC-3′. Those are shown to aid the reader in understanding the fates of various bases under the sequence of Pro-Seq, DNMT, and TAPS conversion steps. As shown, the process includes end repair, A Tailing and ligation to a linking adaptor followed by strand-displacing extension to create a complex of the disclosure (and any optional cleanup).

The complex is treated with DNMT (e.g. DNMT5). In the complex, on the newly-synthesized strand (which is a copy of the “top” or “antisense” strand of the fragment), the DNMT has added a methyl group to the cytosine at the fourth position drawn in the cartoon. This is a feature of mPS of the disclosure in general and hmPS as shown here that is noteworthy. By using the linking adaptor and extension to make the Pro-Seq complex, which includes a sense strand linked to a copy of its corresponding antisense strand, and treating the complex with DNMT, new nucleic acid material is synthesized (the copy of the antisense strand) and mC information from the original fragment is transferred onto the newly synthesized, synthetic nucleic acid of the complex. The complex is subject to treatment with 5′ exonuclease (T7, RecJf) to remove unlinked products, and any optional cleanup.

The remaining product is subject to TAPS mC Conversion in which mC and hmC are converted to DHU (which will be read as T). The product of the TAPS conversion is subject to any amplification and sequencing.

The experiments performed for Example 3 included a process that used mPS v1 with TAPS. As shown in FIG. 4, the experiments included ligating a fragment to a linking adaptor in which a first oligo is linked to a primer annealed to a second oligo, whereby the first oligo is ligated to a first strand of the fragment, extending the primer to make a copy of a second strand of the fragment, resulting in a molecular complex 431 that includes: the first strand ligated to the first oligo, the first oligo linked to the primer wherein the primer is extended into a product that includes a copy of the second strand, and the second strand annealed to the product and ligated to the second oligo. The process included methylating a cytosine base (C) in the copy of the second strand with a reagent composition (e.g., a methyltransferase such as DNMT1 or DNMT5) that acts only on double-stranded DNA and does not modify the single-stranded first strand. The process in Example 3 proceeded as shown in FIG. 5 but using TAPS for converting mC and hydroxymethyl cytosine (hmC) in the complex into dihydrouracil (DHU) leading to base-calling logic e.g., as shown in FIG. 11 and FIG. 62. The methylating step was performed using DNMT5 that methylates unmodified C in hemi-methylated CpG dinucleotides and the converting step uses ten-eleven translocase (TET) enzymes and/or pyridine borane.

It is noted that the DNMT step (as shown in FIG. 61) adds methylation to C in newly-synthesized strands. The information of modified from fragment that existed in a biological sample is enzymatically reproduced onto synthetic DNA strands. It is the epigenetic information that is traced forward from a biological DNA fragment onto a synthetic strand.

For sequencing, the linked strands are used to seed a cluster (FIG. 6) and amplified to form a cluster (FIG. 7) and the cluster is subject to one or more sequencing reactions. In numerous of the examples shown in disclosure, certain figures show a Read 1 and a Read 2 in a “same direction” on the figure, as if those reads were made conducting a sequencing-by-synthesis reaction on strands in a cluster in a same direction. For this Example 3, those reads were taken from opposed ends of strands in a cluster as shown in FIG. 16. The relative position and orientation of the sequence read primer binding and direction of sequencing reaction is flexible and those may be conducted on a cluster in any direction suitable for the workflow of the particular embodiment.

FIG. 62 shows the generation of Read 1 and Read 2 by sequencing and base calling from consensus reads. Again, this processing may be performed by software in a computer system that receives data from a sequencing platform. As shown, two adjacent positions in the first strand may be called as GC when sequencing data read for corresponding positions from the first strand and from the copy of the second strand include GC and GC, respectively, GmC when sequencing data read for corresponding positions from the first strand and from the copy of the second strand include GT and GT, respectively, and GhmC when sequencing data read for corresponding positions from the first strand and from the copy of the second strand include GT and GC, respectively. This illustrates 6-base sequencing (A, C, 5mC, 5hmC, G, and T) and also shows that, by relying on the activity of an enzyme (DNMT) on CpG dinucleotides in duplex DNA, the “calling” of a cytosine in one strand (the “first” strand) uses a dimension of information obtained an adjacent position along the strand.

Looking at what is labeled as “Read 1” in FIG. 62, one observes 3 positions that will be called as a C or modified C in the consensus. Those positions appear in the consensus call as C, mC, and hmC. Those bases were called based on being in CpG dinucleotides and what was found in Read 2.

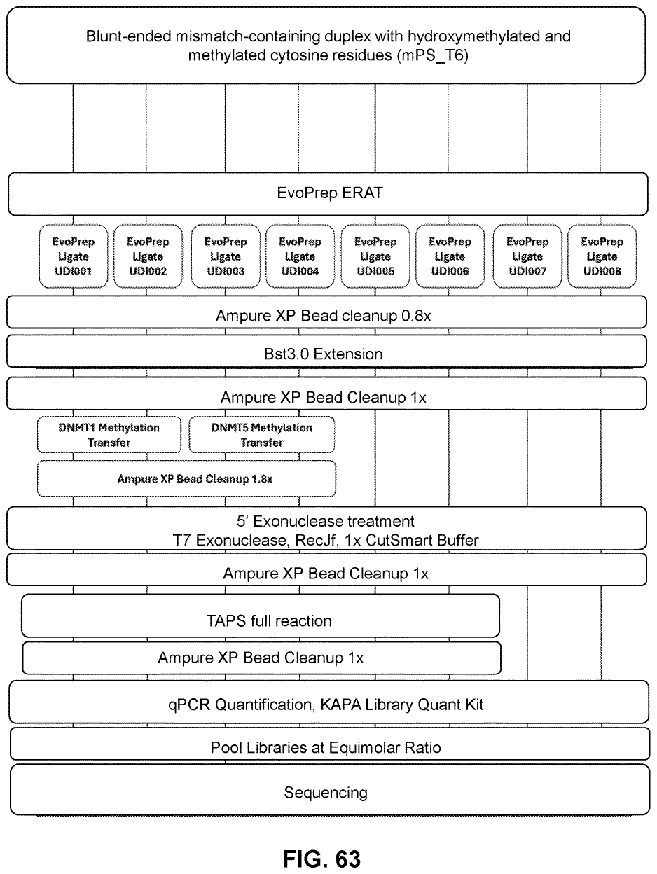

FIG. 63 gives an overview of workflow in this example. This workflow uses the sixth template (mPS t6), discussed above, in eight samples. Samples land 2 are labeled UDI001 and UDI002 and were treated with DNMT1 & TAPS for ‘6-base’ Pro-Seq. Samples 3 and 4 are labeled UDI003 and UDI004 were treated with DNMT5 (instead of DNMT1) & TAPS for ‘6-base’ Pro-Seq. Samples 5 and 6, labeled UDI005 and UDI005, were subject to ‘5-base’ Pro-Seq (mPS) of the disclosure. Samples 7 and 8 (labeled UDI007 and UDI007) were subject to a standard ‘4-base’ Pro-Seq workflow.

### Expected Results:

Samples 1-4 Should distinguish variants at positions 11 and 42 from 27 and 46

Samples 5-6 Should identify variants at positions 11, 27,42 and 46 (but with ‘hmC logic’)

Samples 7-8 should identify no variants

No samples should identify the variant at position 58

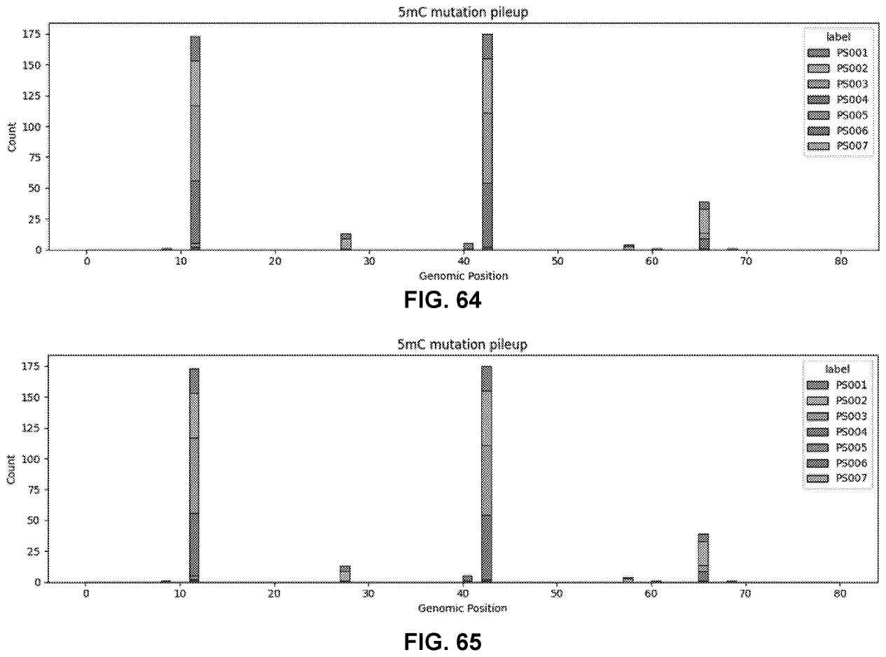

FIG. 64 gives a plot showing mC detection. Using mC consensus logic shown on previous slide (T: T/G: G), the plot shows mC positions in mPS t6. As expected, detection at positions 11 and 42 is achieved by Samples 1-4. No other samples detect variants. Detection at position 46 may be due to non-specific DNMT transfer of mC across from hmC. DNMT can be optimized to reduce off-target transfer

FIG. 65 is a plot showing hmC detection. Using hmC consensus logic shown on previous slide (T: C/G: G), the plot shows hmC positions in mPS t6. As expected, detection at positions 11, 27, 42 and 46 is achieved by Samples 5-6. Samples 1-4 detect hmC at positions 27 and 46 as expected. Some non-specific detection is observed at positions 11 and 42, potentially due to incomplete TAPS conversion. TAPS can be further optimized under these conditions to increase conversion. Here, hemi-hmC detection is not possible with this Example 3 method, as the logic (C: C/G: G) is the same as a regular C base. Collectively, the data show that by combining Pro-Seq workflows (e.g., making complexes representing a sense and an antisense strand of a fragment for sequencing with built-in error correction) with enzymatic methylation treatment according to certain processes of the disclosure, and implementing sequence read consensus determination and base-calling that may be executed by software modules, systems and methods of the disclosure are useful for identifying four canonical bases (A, C, G, and T) and at least two modified bases (5mC and 5hmC) and the positions of those six bases along both strands of a nucleic acid fragment as may be found in a biological sample such as blood or plasma sample obtained by liquid biopsy.

1. A method comprising:
ligating a fragment to a linking adaptor in which a first oligo is linked to a primer annealed to a second oligo, whereby the first oligo is ligated to a first strand of the fragment;
extending the primer to make a copy of a second strand of the fragment, resulting in a molecular complex that includes: the first strand ligated to the first oligo, the first oligo linked to the primer wherein the primer is extended into a product that includes a copy of the second strand, and the second strand annealed to the product and ligated to the second oligo;
methylating a cytosine base (C) in the copy of the second strand with a reagent composition that acts only on double-stranded DNA and does not modify the single-stranded first strand; and
converting mC and hydroxymethyl cytosine (hmC) in the complex into dihydrouracil (DHU).

2. The method of claim 1, wherein the methylating step is performed using a methyltransferase enzyme that methylates unmodified C in hemi-methylated CpG dinucleotides.

3. The method of claim 1, wherein the converting step uses ten-eleven translocase (TET) enzymes and/or pyridine borane.

4. The method of claim 1, further comprising seeding a sequencing reaction with the complex by capturing the first strand and the copy of the second strand with capture primers.

5. The method of claim 4, further comprising conducting the sequencing reaction using a sequencing system connected to an analysis unit to generate sequence data representing the fragment, wherein the analysis unit makes base calls for the first strand based on sequencing data read from the first strand and from the copy of the second strand.

6. The method of claim 5, wherein the analysis unit calls two adjacent positions in the first strand as:
GC when sequencing data read for corresponding positions from the first strand and from the copy of the second strand include GC and GC, respectively,
GmC when sequencing data read for corresponding positions from the first strand and from the copy of the second strand include GT and GT, respectively, and
GhmC when sequencing data read for corresponding positions from the first strand and from the copy of the second strand include GT and GC, respectively.

7. The method of claim 5, wherein the solid support comprises a surface of a flow cell or a surface of a bead.

8. The method of claim 1, wherein after the converting step the first strand and the copy of the second strand are linked together at 5′ regions thereof and have the same sequence of bases in the same order except that C has been converted to DHU in some places.

9. The method of claim 1, further comprising—prior to the ligating step—end-repairing and A-tailing a plurality of fragments that include the fragment obtained from a sample.

10. The method of claim 9, wherein the sample comprises blood or plasma.

11. The method of claim 9, wherein the plurality of fragments are used in a whole genome sequencing processing to identify sequences of, and positions of epigenetic modifications within, the plurality of fragments from the sample.

12. The method of claim 11, wherein positions of epigenetic modifications in tumor DNA from a tumor have previously been determined for a subject from whom the sample was obtained and the method includes detecting the epigenetic modifications among the plurality of fragments from blood or plasma from the sample to detect the tumor DNA in the sample.

13. The method of claim 1, further comprising amplifying the first strand and the copy of the second strand to yield amplification products in which DHU is represented as a T.

14. The method of claim 9, wherein in the amplification products, any unmodified C present in the first strand of the fragment is represent as a C.

15. The method of claim 14, wherein the fragment is from a sample from a subject and method includes identifying patterns of cytosine modification including methylation and hydroxymethylation at genetic loci of the subject.

16. The method of claim 15, wherein the sample is from a tumor and the fragment comprises tumor DNA and the identifying step provides tumor-specific patterns of cytosine modification.

17. The method of claim 16, further comprising comparing the tumor-specific patterns of cytosine modification to a cancer database or atlas for the detection of evidence of cancer in the subject.

18. The method of claim 16, further comprising subsequently assaying a post-treatment sample from the subject for persistence of the patterns of cytosine modification as evidence of minimal residual disease.

19. The method of claim 18, wherein the sample comprises a formalin-fixed, paraffin embedded slice of tumor tissue or a tumor biopsy and the post-treatment sample comprises a liquid biopsy sample and the assaying step comprises analysis of cell-free DNA in the liquid biopsy sample.

20. The method of claim 1, wherein the linking adaptor attaches a first sequencing primer binding site to the first stand and a second sequencing primer binding site to the copy of the second strand and the method includes forming a cluster from the complex and conducting a first sequencing reaction using a first sequencing primer to obtain a first read for the first strand and conducting a second sequencing reaction using a second sequencing primer to obtain a second read for the copy of the second strand.

21-41. (canceled)


SEQUENCE LISTING (10 sequences)

SEQ ID NO: 1 - DNA, 140 bp, synthetic construct
  source 1..140: mol_type = other DNA
ttgagcgcgtaggcttcgatatcgtcggtcgggtcatacgtggacttgtcacgcttgctccactccgtgccgccggactattctcctcactgcggcccatatccacctcaaccggatcgaaggcttcaccggtcatggtg

SEQ ID NO: 2 - DNA, 132 bp, synthetic construct
  source 1..132: mol_type = other DNA
caccatgaccggtgaagccttcgatccggttgaggtggatatgggccgcagtgaggagaatagtccggcggcacggagtggagcaagcgtgacaagtccacgtatgacccgaccgacgatatcgaagcctac

SEQ ID NO: 3 - DNA, 140 bp, synthetic construct
  source 1..140: mol_type = other DNA
acggactttgcccgcctgcaagctgcgtggccacgtcggtgaactgtgcaggcagcatacgcatggcggctttatactgctccccgctttctgtgcagccagcgcctgtcggctcagcgactgttcaacgactgccgctg

SEQ ID NO: 4 - DNA, 116 bp, synthetic construct
  source 1..116: mol_type = other DNA
gtcgttgaacagtcgctgagccgacaggcgctggctgcacagaaagcggggagcagtataaagccgccatgcgtatgctgcctgcacagttcaccgacgtggccacgcagcttgca

SEQ ID NO: 5 - DNA, 140 bp, synthetic construct
  source 1..140: mol_type = other DNA
cgctcagactgcccccgtgttattcgtcgatttggtgccgtaatcgaaactgcctgttgttttcgtcccgtaatcaaacgacgatgtggttttcgtccccaaatccgtaccggatgcactggcactgtgggtgtgcgact

SEQ ID NO: 6 - DNA, 124 bp, synthetic construct
  source 1..124: mol_type = other DNA
tgccagtgcatccggtacggatttggggacgaaaaccacatcgtcgtttgattacgggacgaaaacaacaggcagtttcgattacggcaccaaatcgacgaataacacgggggcagtctgagcg

SEQ ID NO: 7 - DNA, 140 bp, synthetic construct
  source 1..140: mol_type = other DNA
gccaacaacatgccggaacagtacgacgggtacagcaggtagcgcagatcatcaacggtgtgttcagccagttactggcaactttcccggcgagcctggctaaccgtgaccagaacgaagtgaacgaaatccgtcgccag

SEQ ID NO: 8 - DNA, 140 bp, synthetic construct
  source 1..140: mol_type = other DNA
ctggcgacggatttcgttcacttcgttctggtcacggttagccaggctcgccgggaaagttgccagtaactggctgaacacaccgttgatgatctgcgctacctgctgtacccgtcgtactgttccggcatgttgttggc

SEQ ID NO: 9 - DNA, 79 bp, synthetic construct
  source 1..79: mol_type = other DNA
gccagcagcgccgggatttgtggaaggcggagagtcagttcgcggtactggaggaggcggcgcaacgtcgccagctgtc

SEQ ID NO: 10 - DNA, 79 bp, synthetic construct
  source 1..79: mol_type = other DNA
gacagctggcgacgttgcgccgcctcctccagtaccgcgaactgactctccgccttccacaaatcccggcgctgctggc